(19) United States
(12) Patent Application Publication
(10) Pub. No.: US 2024/0252508 A1
(43) Pub. Date: Aug. 1, 2024

(54) Novel glucose uptake inhibitors for use in the treatment of cancer

(71) Applicant: NOVELYEAST BV, Oud-Heverlee (Blanden) (BE)

(72) Inventors: Johan THEVELEIN, Oud-Heverlee (Blanden) (BE); Ward VANTHIENEN, Mechelen (BE)

(73) Assignee: NOVELYEAST BV, Oud-Heverlee (Blanden) (BE)

(21) Appl. No.: 18/553,576

(22) PCT Filed: Apr. 15, 2022
(86) PCT No.: PCT/EP2022/060154

§ 371 Date: Oct. 2, 2023

(30) Foreign Application Priority Data
Apr. 15, 2021 (EP) 21168650.6

(51) Int. Cl. A61K 31/55; A61K 31/519; A61K 31/5377; A61K 31/538; A61P 35/00

(52) U.S. Cl. CPC A61K 31/55; A61K 31/519; A61K 31/5377; A61K 31/538; A61P 35/00

(57) ABSTRACT

The present invention relates to inhibitors of hexokinase-dependent glucose carrier-mediated glucose uptake (Warbicins) that can be used to inhibit proliferation of cancer cells and other cells with an overactive glucose uptake and catabolism, i.e. the Warburg effect. The Warbicins of the invention are used in the prevention or treatment of cancers or other conditions associated with or aggravated by an overactive glycolytic flux, such as pulmonary hypertension, cardiac hypertrophy, heart failure, atherosclerosis, Alzheimer's diseases, multiple sclerosis, polycystic kidney disease, tuberculosis, diabetic kidney disease and autoimmune diseases.

### FIELD OF THE INVENTION

The present invention relates the fields of medicine, particularly oncology and to pharmacy. Specifically, the invention pertains to inhibitors of hexokinase-dependent glucose carrier-mediated glucose uptake that can be used to inhibit proliferation of cancers cells and other cells with an overactive glucose uptake and catabolism, i.e. the Warburg effect. The invention further relates to the use of the inhibitors of the invention for the prevention or treatment of cancers or other conditions associated with or aggravated by an overactive glycolytic flux.

### BACKGROUND OF THE INVENTION

Cancer cells and yeast cells share a preference for fermentation over respiration of glucose even under aerobic conditions and in both cases, this fermentative metabolism is correlated with rapid growth and proliferation^{1,2}. In cancer cells this phenomenon is called the Warburg effect³ and leads to lactic acid production, while in yeast it is called the Crabtree effect and causes ethanol production⁴. Although the Warburg effect has been studied extensively, the primary biochemical cause responsible for the overactive glycolytic flux remains uncertain⁵. A general property of cancer cells is hyperactive glucose uptake which forms the basis for detection of a wide variety of tumor types using 2-¹⁸F-fluoro-2-deoxyglucose and positron emission tomography⁶. Glucose uptake and phosphorylation are also considered to exert major control on glycolytic flux^{7,8,9,10} and glucose transporter, as well as hexokinase overexpression, is a common feature of many cancer types^{10,11,12,13,14,15}. Many studies have provided evidence that the Warburg effect is important for the rapid proliferation and survival of cancer cells¹⁶. Hence, the Warburg effect appears to be a highly promising target for anti-cancer therapies, further supported by its widespread prevalence in cancer cells and its correlation with the aggressiveness of tumors^{9,17,18}.

The yeast Saccharomyces cerevisiae is well known for its high capacity of alcoholic fermentation, being exploited for production of wine, beer and other alcoholic beverages. When exposed to glucose or other related fermentable sugars, it rapidly represses respiration activity at transcriptional and post-translational levels and fully switches to ethanol fermentation, even under fully aerobic conditions¹⁹. In addition, insufficient respiratory capacity results in short-term ‘overflow’ metabolism at the level of pyruvate²⁰. Although multiple crucial genetic components of the glucose repression pathway have been identified, the initial glucose sensing mechanism, in particular the involvement of the major hexokinase of yeast, Hxk2, and the interplay with the glucose sensing mechanisms for activation of the cAMP-protein kinase A (PKA) pathway are not well understood^{21,22}. It is also unclear whether the physiological resemblance between the preference of yeast and cancer cells for fermentation is mimicked at the molecular level by common underlying mechanisms¹. On the other hand, a common mechanism between yeast and cancer cells is the activation of Ras by the glycolytic intermediate fructose-1,6-bisphosphate (Fru1,6bisP), which in both cases may couple increased glycolytic flux to stimulation of cell growth and proliferation²³.

Glucose is taken up in mammalian and yeast cells by low- and high-affinity facilitated diffusion carriers belonging to the Major Facilitator Superfamily^{24,25,26,27} Mammalian GLUT carriers and yeast Hxt carriers share a similar structure with 12 transmembrane domains, cytoplasmic N- and C-termini and they have significant sequence similarity. The presence of a cytosolic ATP-binding domain in the mammalian GLUT1 carrier and its role in inhibition by ATP of glucose uptake has been documented in detail^{28,29,30,31,32} and a similar mechanism has been suggested for GLUT4^{33,34}. The presence and possible role of ATP-binding in yeast Hxts has not been investigated. Several inhibitors of the mammalian GLUT carriers are available and inhibit cancer cell proliferation³⁵. Little is known about their effect on yeast Hxts.

After its uptake, glucose is phosphorylated in mammalian and yeast cells by hexokinase enzymes, which also belong to the same family and show significant sequence similarity³⁶. There is a conspicuous difference between the yeast and mammalian hexokinases with respect to their control by feedback inhibition. Whereas mammalian hexokinases are controlled through feedback-inhibition by their product glucose-6-phosphate (Glu6P), this is not the case for yeast hexokinase^{10,36,37}. The equivalent control has remained elusive until the unexpected discovery that a series of yeast mutants with varying deficiencies for growth on glucose or related fermentable sugars, all carried mutations in TPS1, the gene encoding trehalose-6-phosphate (Tre6P) synthase, the first enzyme of trehalose biosynthesis^{38,39,40,41}. This led to the discovery that Tre6P, made from Glu6P and UDPG, acts as feedback inhibitor of yeast hexokinase³⁷. While the tps1 mutants are highly sensitive to even low mM concentrations of glucose^{23,42}, deletion of HXK2, encoding the most active hexokinase isoenzyme, restores normal growth on glucose⁴³. Addition of glucose to tps1A cells causes dramatic hyperaccumulation of sugar phosphates, especially Fru1,6bisP, and depletion of ATP and downstream glycolytic metabolites^{39,40}. Recent work has provided evidence that the hyperaccumulation of Fru1,6bisP is, at least in part, due to persistent glucose-induced intracellular acidification, which likely compromises glyceraldehyde-3-phosphate dehydrogenase (GAPDH) activity because of its unusually high pH optimum⁴⁴.

Obligatory coupling of sugar transport and phosphorylation is well known from the bacterial phosphotransferase system (PTS)⁴⁵. Although a similar system has not been found in eukaryotic cells, evidence has been reported suggesting optional transport-associated (or vectorial) phosphorylation (TAP) of glucose both in yeast and mammalian cells. In yeast, 2-deoxyglucose pool labelling experiments consistently revealed that under the conditions of these experiments, 2-deoxyglucose entered the cells first in the 2-deoxyglucose-6-phosphate pool and only subsequently in the free 2-deoxyglucose pool^{46,47,48,49,50,51} Although these papers suggested transport-associated phosphorylation of sugar, interpretation of the results has been controversial⁵². The phenomenon received little further attention after the demonstration by a quench-flow technique that high-affinity uptake measured on a sub-second time scale was not dependent on presence of the sugar kinases and the conclusion that glucose entered yeast cells as free sugar⁵³. However, the anaerobic/buffer conditions used in the quench-flow experiments predict very low intracellular ATP levels, which may prevent effective transport-associated phosphorylation at very short time scales. In mammalian cells, transport-associated phosphorylation of 2-deoxyglucose was suggested to occur in adipocytes⁵⁴ and Ehrlich ascites tumor cells⁵⁵. Physical binding of hexokinase to plasma membranes from glioma⁵⁶ and ascites tumor cells⁵⁷ and to GLUT4 in muscle cells⁵⁸ has also been documented suggesting possible coupling between glucose uptake and subsequent intracellular phosphorylation.

Because of the importance of the Warburg effect for proliferation and viability of cancer cells, many studies have explored inhibition of glycolysis for anti-cancer therapy^{59,60,61}. Such drugs, however, generally suffer from adverse side-effects because of the universal importance of glucose catabolism in virtually all cell types. Similar findings have been made with glucose uptake inhibitors^{35,62}.

It is thus an object of the present invention to provide for novel inhibitors of overactive glucose uptake in cancer cells without compromising basal glucose uptake in healthy cells, for use in the treatment of cancer.

### SUMMARY OF THE INVENTION

In a first aspect, the invention pertains to an inhibitor of hexokinase-dependent glucose carrier-mediated glucose uptake for use in the prevention or treatment of a cancer or a condition associated with or aggravated by an overactive glycolytic flux. Preferably, the inhibitor of the invention inhibits the hexokinase-dependent glucose uptake by a glucose carrier that is at least one of a mammalian GLUT carrier and a yeast HXT carrier, wherein more preferably the mammalian GLUT carrier is a class I mammalian GLUT carrier, most preferably at least one of a human GLUT1 and GLUT4 glucose carrier.

In one embodiment, the inhibitor of the invention is characterised in at least one of: a) growth inhibition of A549 lung adenocarcinoma cells grown in a medium with 1 mM glucose at a concentration of the inhibitor of no more than 50 μM; and, b) restoration of growth on glucose of a tps1A yeast strain in a medium containing 2% galactose and 2.5 mM glucose at a concentration of the inhibitor of no more than 100 μM.

In one embodiment, the inhibitor of the invention is characterised in at least one of: a) the structure of the inhibitor comprises a moiety that resembles the structure of adenosine; and, b) the inhibitor binds into the ATP-binding domain of a hexokinase-dependent glucose carrier.

In one embodiment, the inhibitor of the invention is for a use wherein the cancer is a solid tumor or a blood malignancy. In one embodiment, the cancer is a newly diagnosed cancer that is naive to treatment, a relapsed cancer, a refractory cancer, a relapsed and refractory cancer and/or metastasis of the cancer.

In one embodiment, the inhibitor of the invention is for a use wherein the inhibitor is used in the prevention and/or treatment of the cancer or metastasis thereof as adjunctive therapy, in combination with one or more treatments selected from the group consisting of: surgery, radiation therapy, chemotherapy and immunotherapy.

In one embodiment, the inhibitor of the invention is for a use wherein the condition associated with or aggravated by an overactive glycolytic flux is a condition or disease selected from the group consisting of pulmonary hypertension, cardiac hypertrophy, heart failure, atherosclerosis, Alzheimer's diseases, multiple sclerosis, polycystic kidney disease, tuberculosis, diabetic kidney disease and an autoimmune disease.

In one embodiment, the inhibitor of the invention is a compound of the general formula (I):

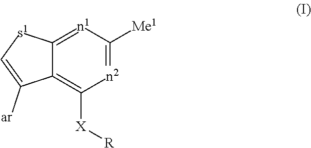

wherein
each of s¹, n¹, and n² is independently chosen from N, O, and S;
Me¹ is a C₁₋₁₀hydrocarbon moiety that is optionally substituted with 1 or 2 alkyl, halogen, or alkoxy moieties;
ar is a 5-10-membered aryl or heteroaryl moiety that is optionally substituted with 1 or 2 alkyl, halogen, or alkoxy moieties;
X is S, NH, or O; and
R is a C₁₋₂₅hydrocarbon moiety that can comprise 0 to 8 heteroatoms and 0 to 3 cyclic moieties.

In one embodiment, the inhibitor of the invention is a compound of general formula (II):

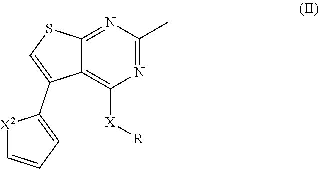

wherein X² is S, NH, or O, and wherein X and R are as defined in claim 9.

In one embodiment, the inhibitor of the invention is a compound of general formula (III):

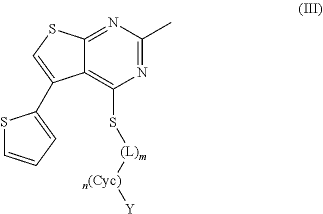

wherein
m is 0, 1, or 2, preferably 0 or 1;
n is 0, 1, or 2, preferably 0 or 1;
L is a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N;
Cyc is a 5 to 10 membered cyclic, heterocyclic, aromatic, or heteroaromatic moiety that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N; and
Y is H or a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N.

In one embodiment, the inhibitor of the invention is a compound of the general formula (Ia) or (Ib) or (Ic):

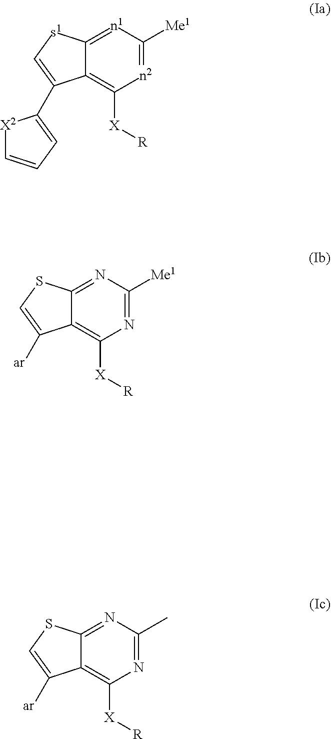

In one embodiment, the inhibitor of the invention is a compound of the general formula (IIa):

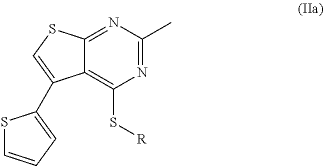

In one embodiment, the inhibitor of the invention is a compound wherein R is of general formula (R1):

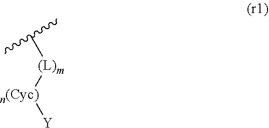

In one embodiment, the inhibitor of the invention is a compound wherein R is selected from the group consisting of R1-R60 of Table 1.

### DESCRIPTION OF THE FIGURES

FIG. 1. Reducing glucose transport in the tps1A strain rescues growth on glucose.

a. Spot assay displaying the glucose sensitivity of different hxtA mutants in the tps1A background. Cells were spotted in five-fold dilutions on plates containing 3% glycerol supplemented with the indicated glucose concentrations. b. Uptake of 2.5 mM glucose was measured in tps1A and tps1A hxtA strains. One-way ANOVA statistical analysis showed significant reduction in glucose uptake when comparing the effect of additional HXT deletion (**, p<0.01; ***, p<0.001). c. Kinetic analysis for glucose uptake of tps1A and tps1A hxt6,7,2,4,5Δ cells. d.-f. metabolite profiles for d. Glu6P, e. Fru1,6bisP and f. ATP were measured after addition of 10 mM glucose to tps1Δ and tps1Δ hxt6,7,2,4,5Δ cells. For all experiments, cells were (pre)grown on YP medium containing 3% glycerol.

FIG. 2. WBC-A rescues growth on glucose and normalizes glycolytic metabolite deregulation in tps1Δ cells.

a. Chemical structure of WBC-A. b-e. Growth of tps1Δ cells on 2% galactose supplemented with different glucose concentrations. Cells were treated with either DMSO (black lines), 12.5 μM WBC-A (red lines), 25 μM WBC-A (orange lines), 50 μM WBC-A (light blue lines) or 100 μM WBC-A (dark blue lines). Metabolic profiles are shown for Glu6P (f, h, j) and Fru1,6bisP (g, i, k) accumulation as a function of time after addition of glucose at time point zero. Compounds were added at −10 min. f, g tps1Δ cells were given 2.5 mM glucose in the absence (black circles) or presence of 10 μM (red squares), 25 μM (orange triangles) or 100 μM WBC-A (blue diamonds). h, i tps1Δ cells were treated with DMSO (closed symbols) or 25 μM WBC-A (open symbols) after which 2.5 mM glucose (circles) or 7.5 mM glucose (squares) was added at time zero. j, k Wild type cells were given 2.5 mM glucose in the absence (closed circles) or presence of 25 μM WBC-A (open circles). For all experiments, cells were (pre)grown in Complete Synthetic medium containing 2% galactose. Cells were resuspended in the same medium for 30 min prior to glucose addition.

FIG. 3. WBC-A inhibits glucose uptake of wild type and tps1Δ cells with mixed type inhibition.

a. Dose-response inhibition of 2.5 mM glucose uptake by WBC-A in wild type (closed circles) and tps1Δ (open circles) cells. IC₅₀ values are indicated by dashed lines. b. Graphical representation of mixed type inhibition. c. Kinetic analysis of glucose uptake in wild type cells in the absence (black circles) or presence of 25 μM (red squares) or 50 μM WBC-A (blue diamonds). Corresponding V(′)max (dashed lines) and K(′)_{M} (open circles) values are indicated. d. Lineweaver-Burk plot analysis using the data of c for wild type cells for the determination of mode-of-inhibition. e. Corresponding Dixon plot analysis for estimation of the Kᵢ (closed red circle) and K′; (closed blue circle) inhibitor constants of WBC-A. For all experiments, cells were (pre)grown on Complete Synthetic medium containing 2% galactose.

FIG. 4. Structural analogs of WBC-A rescue growth and inhibit glucose uptake of tps1Δ cells to varying degrees.

a. Structural analogs of WBC-A that rescued growth of the tps1Δ strain were compared for their influence on maximal growth rate relative to the control (DMSO). Compounds were added at 100 μM to tps1Δ cells growing in medium containing 2% galactose or 2% galactose supplemented with 2.5 mM glucose. b. Inhibition of 2.5 mM glucose (black bars) or 2.5 mM galactose (grey bars) uptake in tps1Δ cells by 25 μM of various WBC compounds. c. Dose-response inhibition of 2.5 mM glucose transport in wild type cells by WBC-A (closed circles) and WBC-2C (open circles). IC₅₀s are indicated by dashed lines. d. Lineweaver-Burk plot analysis for determination of mode-of-inhibition by WBC-2C using the data of c for wild type cells. e. Corresponding Dixon plot analysis for estimation of the Kᵢ(closed red circle) and K′; (closed blue circle) inhibitor constants of WBC-2C. For all experiments, cells were (pre)grown on Complete Synthetic medium containing 2% galactose.

FIG. 5. WBC-A inhibits GLUT activity and growth and glucose uptake of A549 lung adenocarcinoma cells.

a. Dose-response inhibition of 2.5 mM glucose transport by WBC-A in RE700A hxt⁰ gal2Δ cells expressing either pHXT7 (closed circles) or pGLUT1^{V69M} (open circles). b. Dose-response inhibition of 2.5 mM glucose transport by WBC-A in EBY.VW4000 hxt⁰ erg4Δ cells expressing either pHXT7 (closed circles) or pGLUT4^{V85M} (open circles). IC₅₀s in a, b are indicated by dashed lines. c, d. Growth analysis of A549 lung adenocarcinoma cells cultured in RPMI containing either c. 1 mM glucose or d. 5 mM glucose. Cells were treated with 0.1% DMSO (black lines), 12.5 μM WBC-A (red lines), 25 μM WBC-A (orange lines) or 50 μM WBC-A (blue lines). Growth is expressed as fold increase in measured confluency. e. Graphical representation of non-competitive inhibition. f. Kinetic analysis of 2-deoxyglucose uptake in A549 lung adenocarcinoma cells in the absence (black circles) or presence of 50 μM WBC-A (red squares) or 100 μM WBC-A (blue diamonds). Corresponding V(′)max (dashed lines) and K(′)_{M} (open circles) values are indicated. g. Lineweaver-Burk plot analysis using the data of f for determination of mode-of-inhibition. h. Corresponding Dixon plot analysis for estimation of the Kᵢ=K′ᵢ (closed red circle) inhibitor constants of WBC-A. To measure glucose uptake by yeast cells expressing GLUT isoforms, cells were (pre)grown in YP medium with 2% maltose. A549 cells were precultured in RPMI medium supplemented with 10 mM glucose.

FIG. 6. Structural analogs of WBC-A inhibit cell proliferation of different human cancer cell lines.

a. The ratio of IC₅₀ values for inhibition of A549 lung adenocarcinoma cell proliferation on 10 mM and 1 mM glucose is illustrated for every analog that was isolated from the pre-screening (n=77, Supplementary FIG. 5). Phloretin (dark blue bar) was included as a positive control whereas WBC-A (green bar) served as cut-off threshold for selection of compounds with similar or higher potency. b. Maximal growth rates on 1 mM glucose are shown for A549 cells at 6.25 μM (black bars), 12.5 μM (red bars), 25 μM (orange bars) or 50 μM (blue bars) of either WBC-A, -15C, -4C or -11C. Absence of growth is indicated by an asterisk symbol. Growth was set relative to growth on DMSO (100%). c. Phase contrast microscopy images of A549 cells treated with either DMSO or 50 μM of WBC-A, -15C, -4C or -11C. Pictures were taken after three days. d. Cell count of KMS-12-PE (black bars) multiple myeloma cells after 4 days of growth on RPMI medium with 1 mM glucose. Cells were treated with either DMSO or 25 μM of WBC-A, -15C, -4C or -11C. e. Growth curve of the MCF10A breast epithelia cell line transformed with the H-RASV¹² allele on 1 mM glucose. Cells were treated with either DMSO (black line), or 25 μM of WBC-A (red line), WBC-15C (orange line), WBC-4C (light blue line) or WBC-11C (dark blue line). Growth was based on increase in confluency as determined by the Incucyte software. f. Relative fluorescent object counts originating from apoptosis induction in MCF10A H-RASV¹² cells growing on 1 mM glucose, as determined by the Incucyte software. Cells were treated with either DMSO or 25 μM of WBC-A, -15C, -4C or -11C. Fluorescent object counts after three days were corrected for total confluency percentage and normalized to the DMSO control.

FIG. 7. Kinetic characterization of 2-deoxyglucose transport inhibition in A549 lung adenocarcinoma cells by WBC-15C, -4C and -11C.

Inhibition of a. Glucose consumption and b. lactate secretion rates by 25 μM of WBC-A, -15C, -4C and -11C in comparison with the DMSO control for A549 cells incubated in medium supplemented with 1 mM glucose for 8 h. c, d. Graphical representation of competitive and uncompetitive inhibition, respectively. Molecular structures of WBC-15 (e.), WBC-4C (i.) and WBC-11C (m.). Kinetic analysis of 2-deoxyglucose uptake inhibition in A549 cells in the absence (black circles) or presence of 25 μM (red squares) or 50 μM (blue diamonds) WBC-15 (f.), -4C (j.) and -11C (n.). Corresponding V(′)max (dashed lines) and K(′)_{M} (open circles) values are indicated. Lineweaver-Burk plot analysis, shown in g., k. and o., using the data of f., j., n., respectively, for determination of mode-of-inhibition. Inhibitor constants (closed red circles) were estimated using Dixon plot analysis for WBC-15C (h.) and WBC-4C (I.), whereas the Cornish-Bowden plot was applied for WBC-11C (p.). Significance was determined by one-way ANOVA followed by Dunnett's multiple comparisons test (***, p<0.001).

FIG. 8. Physical evidence of transport-associated phosphorylation and the influence of WBC-A.

a. Pulldown of Hxt7-HA from wild type cell extracts by GST-Hxk2. Presence of Hxt7-HA was visualized by western blotting (upper panel), whereas presence of GST and GST-Hxk2 was confirmed by Coomassie blue staining (lower panel). b. Fluorescence microscopy images of BiFC interactions between Hxt7 and Hxk2, Hxk1 or Glk1 at the level of the plasma membrane as well as the cytosolic localization of the three sugar kinases fused to full-length Citrine. c. Spot assay displaying the glucose sensitivity of hxk2Δ hxk1Δ glk1Δ tps1Δ cells transformed with a vector with either no insert, a HXK2 allele or a NLS-HXK2 allele. All plates contained 2% galactose and were supplemented with the indicated glucose concentration. Pictures were taken after three days. d. Uptake inhibition of 1 mM glucose by DMSO or different concentrations of WBC-A was measured in HXK2 hxk1Δ glk1Δ (black bars) and hxk2Δ hxk1Δ glk1Δ (grey bars) cells. e. Uptake rates in d. are put relative to DMSO treated cells (100%) for each strain. f. Uptake rates of glucose (black bars) and fructose (grey bars) at tracer concentration were measured in hxk2Δ hxk1Δ GLK1 cells. DMSO, 250 μM CCCP and 50 μM WBC-A were given 10 min prior to the uptake measurement except for 100 mM maltose which was given simultaneously with the tracer sugar. Significance was determined by two-way ANOVA followed by Sidak's multiple comparisons test (***, p<0.001; ns, non-significant). For all experiments, cells were (pre)grown on medium with 3% glycerol and 2% ethanol except for the spot assay where cells were (pre)grown on 2% galactose in uracil-deficient medium for plasmid retention.

FIG. 9. Protein sequence alignment of the ATP binding domains present in human GLUT compared with the corresponding sequences in yeast Hxt glucose transporters.

The ATP binding domains described for the GLUT1 and GLUT4 carriers are shown first, followed by alignment with the corresponding domains in human GLUT2, 3 and 14 from the same GLUT subfamily, and in the yeast Hxt transporters Hxt1, 2, 3, 4, 5, 6, 7 and Gal2. Fully conserved residues are indicated in red with shading in yellow, while conserved substitutions are indicated in orange with shading in pink.

FIG. 10. Kinetic characterization of WBC-A inhibition of glucose uptake in tps1Δ cells.

a. Kinetic analysis of glucose uptake in tps1Δ cells in the absence (black circles) or presence of 25 μM WBC-A (red squares) or 50 μM WBC-A (blue diamonds). Corresponding V(′)max (dashed lines) and K(′)_{M} (open circles) values are indicated. b. Lineweaver-Burk plot analysis for the determination of mode-of-inhibition. c. Corresponding Dixon plot analysis for estimation of the Kᵢ(closed red circle) and K′; (closed blue circle) inhibitor constants of WBC-A. For all experiments, cells were (pre)grown on Complete Synthetic medium containing 2% galactose.

FIG. 11. Hexokinase activity in extracts of wild type cells is unaffected by WBC-A.

Hexokinase activity was measured in extracts of wild type cells grown on 2% galactose. The activity in the presence of DMSO as control (closed circles) or 50 μM WBC-A (open circles) was determined with a. different glucose concentrations and a fixed ATP concentration of 5 mM and b. different ATP concentrations and a fixed glucose concentration of 5 mM.

FIG. 12. General effect of WBC compounds on wild type growth.

WBC compounds that rescued growth of tps1Δ cells on glucose were compared for their effect at 100 μM on wild type growth on 2% glucose (black bars) and 3% glycerol+2% ethanol (grey bars). Maximal growth rates were determined after 2 days of growth in Complete Synthetic liquid medium.

FIG. 13. WBC-55A has a different structure and action mechanism.

a. Molecular structure of WBC-55A. Metabolic profiles for a. Glu6P and b. Fru1,6bisP accumulation after addition of glucose. At time point zero, 2.5 mM glucose was added to tps1Δ cells in the absence (black circles) or presence of 100 μM WBC-A (red squares) or 100 μM WBC-55A (blue diamonds). Inhibitors were added at −10 min.

FIG. 14. Prescreening of the structural analog library of WBC-A on A549 cells.

The analog library (n=203) was screened for inhibition of growth of A549 cells on RPMI medium supplemented with 1 mM glucose. DMSO (green bar) and WBC-A (orange bar) served as negative and positive control, respectively. In addition, the reference glucose transport inhibitors STF-31, Fasentin, BAY-876, WZB-117 and Cytochalasin B were included and indicated in red. Compounds were added at 50 μM concentration. Number of cells was determined by counting nuclei stained by Hoechst after three days of growth.

FIG. 15. Warbicins affect glucose uptake in the KMS-12-PE multiple myeloma cell line.

Glucose consumption (a.) or lactate secretion (b.) rates are shown for KMS-12-PE cells incubated in RPMI medium supplemented with 1 mM glucose for 8 h Significance was determined by one-way ANOVA with Dunnett's multiple comparisons test (***, p<0.001).

FIG. 16. The free C-terminal part of Citrine does not spontaneously assemble with Hxt7-NCitr.

Fluorescence microscopy images to assess spontaneous BiFC self-assembly. Hxt7-Citrine cells transformed with the empty plasmid show the expected localization of Hxt7 at the plasma membrane (left image). Hxt7-NCitr cells transformed with vector-expressed full-length Citrine show correct expression of Citrine in the cytosol (middle image). Hxt7-NCitr cells transformed with vector-expressed CCitr do not show any fluorescence (right image: fluorescence and DIC). Cells were grown on YP medium supplemented with 3% glycerol and 2% ethanol.

FIG. 17. Cytosolic and nuclear localized hexokinase restores glucose growth of the hxk⁰ mutant.

a. Fluorescent microscopic images of hxk2Δ hxk1Δ glk1Δ cells transformed with a vector expressing either a HXK2 or a NLS-HXK2 allele. b. Spot assay for growth on 2% galactose or different levels of glucose of hxk2Δ hxk1Δ glk1Δ cells transformed with a vector containing either no insert, a HXK2 or a NLS-HXK2 allele. Pictures were taken after 3 days. For every experiment, cells were pregrown on 3% glycerol and 2% ethanol in uracil-deficient medium for plasmid retention.

FIG. 18. Inhibition of fructose and galactose uptake by WBC-A in strains with and without functional hexokinase activity.

a. Inhibition of 1 mM fructose uptake by HXK2 hxk1Δ glk1Δ and hxk2Δ hxk1Δ GLK1 cells treated with either DMSO (black bars) or 50 μM WBC-A (grey bars). c. Inhibition of 1 mM galactose uptake by wild type, gal80Δ, gal80Δ gal3Δ, gal80Δ gal1Δ, gal80Δ gal1Δ gal3Δ cells treated with either DMSO (black bars) or 50 μM WBC-A (grey bars). Uptake rates of a. and c. were set relative to the DMSO control (100%) in b. and d. for each strain, respectively. Significance was determined by two-way ANOVA with Sidak's multiple comparisons test (**, p<0.01; ***, p<0.001; ns, non-significant).

FIG. 19. Effect of known mammalian glucose uptake inhibitors on glucose transport by Hxt7 and GLUT1 expressed in yeast.

Inhibition of 2.5 mM glucose uptake in hxt⁰ gal2Δ cells expressing either a HXT7 (black bars) or GLUT1^{V69M} allele. Cells were treated with either DMSO or 25 μM of WBC-A, Fasentin, STF-31, Cytochalasin B, WZB-117 and BAY-876. Significance is determined by two-way ANOVA followed by Sidak's multiple comparisons test (***, p<0.001). Cells were grown on rich medium containing 2% maltose.

FIG. 20. Evaluation of Warbicin toxicity: weight loss.

Weight loss was determined during 20 days in nude mice treated with WBC-A, WBC-15C, WBC-4C and WBC-11C by daily intraperitoneal injection. a. 20 mg/kg, b. 10 mg/kg and c. 5 mg/kg. Three mice were used for each dose. Standard deviation is shown. No significant difference (p>0.05) between vehicle and compound treated mice was observed across all tested concentrations by applying one-way ANOVA statistical analysis.

FIG. 21. Evaluation of Warbicin toxicity: blood glucose level.

The blood glucose level was determined from sera samples collected post mortem after 20 days of treatment with WBC-A, WBC-15C, WBC-4C and WBC-11C by daily intraperitoneal injection. a. 20 mg/kg, b. 10 mg/kg and c. 5 mg/kg. Three mice were used for each dose. Standard deviation is shown. No significant difference (p>0.05) between vehicle and compound treated mice was observed across all tested concentrations by applying one-way ANOVA statistical analysis.

FIG. 22. Evaluation of Warbicin toxicity: AST/ALT ratio.

To evaluate liver toxicity, the AST/ALT ratio was determined in sera samples collected after 20 days at the end of the experiment from the nude mice treated with WBC-A, WBC-15C, WBC-4C or WBC-11C by daily intraperitoneal injection. a. 20 mg/kg, b. 10 mg/kg and c. 5 mg/kg. Three mice were used for each dose. Standard deviation is shown. No significant difference (p>0.05) between vehicle and compound treated mice was observed across all tested concentrations by applying one-way ANOVA statistical analysis.

FIG. 23. Warbicins affect growth and glucose uptake of the U266 multiple myeloma cell line.

a. Cell count of the U266 multiple myeloma cell line after 4 days of growth on RPMI medium with 1 mM glucose. Cells were treated with either DMSO or 25 μM of WBC-A, -15C, -4C or -11C. Glucose consumption (b.) or lactate secretion (c.) rates are shown for U266 cells incubated in RPMI medium supplemented with 1 mM glucose for 8 h.

FIG. 24. Inhibitory effect of Warbicin® A (A), Warbicin® 4C (B) and Warbicin® 11C (C) on tumor volume growth in a mouse xenograft model relative to the tumor volume at day zero during 10 days of treatment with different concentrations of the Warbicin® compounds as indicated. Figures A, B and C are from the same data set and for reasons of comparison the data for “vehicle” are reproduced in each of the figures A, B and C.

FIG. 25. Body weight changes during the treatment with Warbicin® A (A), Warbicin® 4C (B) and Warbicin® 11C (C) in a mouse xenograft model relative to body weight at day zero during 10 days of treatment with different concentrations of the Warbicin® compounds as indicated. Figures A, B and C are from the same data set and for reasons of comparison the data for “vehicle” are reproduced in each of the figures A, B and C.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the methods and materials described.

For purposes of the present invention, the following terms are defined below.

As used herein, the singular forms “a,” “an” and “the” include plural referents unless the context clearly dictates otherwise. For example, a method for administrating a drug or an agent includes the administrating of a plurality of molecules (e.g. 10's, 100's, 1000's, 10's of thousands, 100's of thousands, millions, or more molecules).

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

As used herein “cancer” and “cancerous”, refer to or describe the physiological condition in mammals that is typically characterized by unregulated cell growth. Cancer is also referred to as malignant neoplasm.

As used herein, “in combination with” is intended to refer to all forms of administration that provide a first drug together with a further (second, third) drug. The drugs may be administered simultaneous, separate or sequential and in any order. Drugs administered in combination have biological activity in the subject to which the drugs are delivered.

As used herein “simultaneous” administration refers to administration of more than one drug at the same time, but not necessarily via the same route of administration or in the form of one combined formulation. For example, one drug may be provided orally whereas the other drug may be provided intravenously during a patient's visit to a hospital. Separate includes the administration of the drugs in separate form and/or at separate moments in time, but again, not necessarily via the same route of administration. Sequentially indicates that the administration of a first drug is followed, immediately or in time, by the administration of the second drug.

A used herein “compositions”, “products” or “combinations” useful in the methods of the present disclosure include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs (alone or in combination) and one or more suitable pharmaceutically acceptable excipients.

As used in the context of the invention, the terms “prevent”, “preventing”, and “prevention” refers to the prevention or reduction of the recurrence, onset, development or progression of a cancer, preferably a cancer as defined herein, or the prevention or reduction of the severity and/or duration of the cancer or one or more symptoms thereof.

As used in the context of the invention, the terms “therapies” and “therapy” can refer to any protocol(s), method(s) and/or agent(s), preferably as specified herein below, that can be used in the prevention, treatment, management or amelioration of cancer, preferably a cancer as defined herein below, or one or more symptoms thereof.

As used herein, the terms “treat”, “treating” and “treatment” refer to the reduction or amelioration of the progression, severity, and/or duration of a cancer, preferably a cancer as defined herein below, and/or reduces or ameliorates one or more symptoms of the disease.

As used herein, “an effective amount” is meant the amount of an agent required to ameliorate the symptoms of a disease relative to an untreated patient. The effective amount of active agent(s) used to practice the present invention for therapeutic treatment of a cancer varies depending upon the manner of administration, the age, body weight, and general health of the subject. Ultimately, the attending physician or veterinarian will decide the appropriate amount and dosage regimen. Such amount is referred to as an “effective” amount. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

### DETAILED DESCRIPTION OF THE INVENTION

Cancer and yeast cells share preference for fermentation over respiration. The present inventors used a yeast tps1Δ mutant, which undergoes apoptosis due to hyperactive glucose uptake and catabolism, to screen for compounds that restore growth of the mutant on glucose. In this screen they identified Warbicin A, an inhibitor of hexokinase-dependent glucose carrier-mediated glucose uptake. Warbicin A and specific structural analogs inhibit glucose uptake by yeast Hxt and mammalian GLUT carriers with compound-specific kinetics. Warbicins inhibit proliferation and trigger cell death in cancer cells in a concentration-dependent manner. Appropriate concentrations show no toxicity in mice. Warbicins target the Warburg effect, directly counteracting overactive glucose uptake and catabolism. As such Warbicins are useful in the treatment of cancers and other conditions associated with or aggravated by an overactive glycolytic flux.

### Inhibitors of the Invention

In a first aspect, therefore, the invention pertains to an inhibitor of hexokinase-dependent glucose carrier-mediated glucose uptake. In one embodiment, the inhibitor of the invention is for use in the prevention and/or treatment of a cancer or a condition associated with or aggravated by an overactive glycolytic flux.

In one embodiment, the inhibitor of the invention, inhibits the hexokinase-dependent glucose uptake by a glucose carrier that is at least one of a mammalian GLUT carrier and a yeast HXT carrier. In one embodiment, the inhibitor of the invention, inhibits the hexokinase-dependent glucose uptake by a glucose carrier that is a class I mammalian GLUT carrier, preferably a human class I GLUT carrier. Thus in a preferred embodiment, the inhibitor of the invention, inhibits the hexokinase-dependent glucose uptake by a glucose carrier that is at least one of a mammalian GLUT1, GLUT2, GLUT3, GLUT4 and GLUT14 glucose carrier, more preferably at least one of a human GLUT1, GLUT2, GLUT3, GLUT4 and GLUT14 glucose carrier, and most preferably at least one of a human GLUT1 and GLUT4 glucose carrier.

In one embodiment, the ability of an inhibitor of the invention to inhibit hexokinase-dependent glucose uptake by a mammalian or human glucose carrier is assayed by heterologous expression of the mammalian or human glucose carrier in a hxt⁰ S. cerevisiae strain that is deficient in glucose uptake due to absence of all endogenous active glucose transporters, and assaying the dose-dependent effect of the inhibitor on glucose transport, e.g. as described in Example 1.5 herein.

More specifically, the inhibition of a GLUT1 carrier can be assayed by expression of a human GLUT1^{V69}M carrier (or corresponding other mammalian carrier) in a hxt⁰ gal2Δ yeast strain (e.g. RE700A) and the inhibition of a GLUT4 carrier can be assayed by expression of a human GLUT4^{V85M} carrier (or corresponding other mammalian carrier) in a hxt⁰ erg4Δ yeast strain (e.g. EBY.VW4000).

In one embodiment, the ability of an inhibitor of the invention to inhibit hexokinase-dependent glucose uptake by a yeast HXT carrier is assayed by testing the ability of the inhibitor to restore growth on glucose of a tps1Δ yeast strain, e.g. as described in Examples 1.2 to 1.4. An inhibitor is capable of restoring growth of a tps1Δ yeast strain is that growth rate of the strain on a medium containing glucose (in addition to another carbon source, e.g. galactose) is higher than the growth rate of the same strain grown under identical conditions in the absence of glucose. The tps1Δ yeast strain preferably is a tps1Δ S. cerevisiae strain. The ability of the inhibitor to restore growth on glucose is preferably tested in a liquid medium (e.g. YP medium) containing 2% galactose and 2.5-5 mM glucose. In one embodiment, the inhibitor restores the growth of the tps1Δ yeast strain in a medium containing 2% galactose and 2.5 mM glucose at a concentration of the inhibitor of no more than 100, 80, 60, 50, 45, 40, 35, 30, 25, 20, 15, 12.5, 10, 7.5, 5, 2, 1, 0.5, 0.2 or 0.1 μM.

In one embodiment, the ability of an inhibitor of the invention to inhibit hexokinase-dependent glucose uptake by glucose carrier is assayed by determining the inhibitor's dose-dependent inhibition of proliferation of a cancer cell.

In one embodiment, the ability of an inhibitor of the invention to inhibit the proliferation of a cancer cell is assayed by in vitro culture of a cancer cell in a suitable tissue culture medium comprising glucose (as carbon source) in the presence of the inhibitor and comparing the growth rates of the cancer cell grown in the presence of the inhibitor with the growth rate of the same cancer grown under identical conditions in the absence of the inhibitor, e.g. as described in Example 1.5 herein. Preferably, a range of different concentrations of the inhibitor is assayed so as to determine the dose-dependency of the inhibition of proliferation of a cancer cell by the inhibitor. In principle any cancer cell that is amenable to in vitro tissue culture can be used to assay the ability of an inhibitor of the invention to inhibit the proliferation of a cancer cell. The person skilled in the art will know suitable publicly available cancer cell lines that can be used for the assay, such as those used in the Examples herein: the A549 lung adenocarcinoma cell line, the multiple myeloma KMS-12-PE cell line or the MCF10Δ breast epithelia cell line transformed with the H-RAS^{V12} allele. In one embodiment, the inhibitor inhibits the growth of A549 lung adenocarcinoma cells grown in a medium with 1 mM glucose at a concentration of the inhibitor of no more than 50, 45, 40, 35, 30, 25, 20, 15, 12.5, 10, 7.5, 5, 2, 1, 0.5, 0.2 or 0.1 μM.

In one embodiment, the inhibitor of the invention is a non-competitive inhibitor of hexokinase-dependent glucose carrier-mediated glucose uptake, such as WBC-A or WBC4C as disclosed in the Examples herein.

In one embodiment, the inhibitor of the invention is a competitive inhibitor of hexokinase-dependent glucose carrier-mediated glucose uptake, such as WBC-15C as disclosed in the Examples herein.

In one embodiment, the inhibitor of the invention is an uncompetitive inhibitor of hexokinase-dependent glucose carrier-mediated glucose uptake, such as WBC-11C as disclosed in the Examples herein.

The type of inhibition of an inhibitor of the invention can be determined using Lineweaver-Burk plot analysis for the kinetics of 2-deoxyglucose uptake inhibition by an inhibitor of the invention in e.g. cancer cells such as A549 lung adenocarcinoma cells, as described in Examples 1.5 and 1.7 herein.

In one embodiment, the structure of the inhibitor of the invention comprises a moiety that resembles the structure of adenosine. Adenosine is a 6,9-disubstituted purine. It was found that suitable inhibitors conserve the bicyclic heteroaromatic moiety of adenosine, as well as the 9-substitution. Preferred inhibitors are substituted purines, substituted 5,7-diazaindoles, and substituted 5,7-diazabenzothiophenes, of which substituted 5,7-diazabenzothiophenes are most preferred. Preferred substitutions (wherein the numbering refers to numbering as for indole or for benzothiophene) are 3-substitutions, 4-substitutions, and 6-substitutions, wherein more preferably all three are present. A preferred 6-substitution is a methyl or ethyl substitution, preferably a methyl. The 4-substitution is widely tolerated; a preferred 4-substitution is —S—R or —O—R wherein R is as described later herein, preferably —S—R. A preferred 3-substitution is a 5- or 6-membered aryl or heteroaryl moiety, preferably a heteroaryl moiety, more preferably a 5-membered moiety, most preferably a 5-memebered heteroaryl moiety. Suitable 5-membered heteroaryl moieties are thiophene, furane, and pyrrole, which are preferably 2-linked, and of which thiophene is preferred.

Accordingly highly preferred structures that resemble adenosine are at least one of: i) 3-thiophen-2-yl-6-methyl-5,7-diazaindoles;
ii) 3-thiophen-2-yl-6-methyl-5,7-diazabenzothiophenes;
iii) 4-substituted-3-thiophen-2-yl-6-methyl-5,7-diazaindoles; and,
iv) 4-substituted-3-thiophen-2-yl-6-methyl-5,7-diazabenzothiophenes, of which ii) and iv) are more preferred, and iv) is most preferred, particularly when the 4-substitution is —S—R.

In one embodiment, the inhibitor of the invention binds into the ATP-binding domain of a hexokinase-dependent glucose carrier, wherein the hexokinase-dependent glucose carrier preferably is a glucose carrier as defined above. In one embodiment, the inhibitor binds into the ATP-binding domain of a hexokinase-dependent glucose carrier with a dissociation constant Kᵢ of no more than 100, 80, 60, 50, 45, 40, 35, 30, 25, 20, 15, 12.5, 10, 7.5, 5, 2, 1, 0.5, 0.2 or 0.1 μM.

In one embodiment, the inhibitor binds the hexokinase-dependent glucose carrier with an dissociation constant K′ᵢ of no more than 100, 80, 60, 50, 45, 40, 35, 30, 25, 20, 15, 12.5, 10, 7.5, 5, 2, 1, 0.5, 0.2 or 0.1 μM. Kᵢ and K′ᵢ are herein understood to be the dissociation constants for binding of the inhibitor to the hexokinase-dependent glucose carrier, or for binding of the inhibitor to the hexokinase-dependent glucose carrier with its ATP substrate bound, respectively.

In one embodiment, an inhibitor of the invention is a compound of the general formula (I):

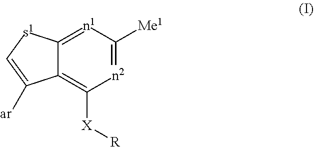

wherein
each of s¹, n¹, and n² is independently chosen from N, O, and S; preferably s¹ is S. Preferably n¹ is N. Preferably n² is N. Preferably n¹ and n² are both N. Most preferably n¹ and n² are both N and s¹ is S.
Me¹ is a C₁₋₁₀hydrocarbon moiety that is optionally substituted with 1 or 2 alkyl, halogen, or alkoxy moieties; preferred hydrocarbon moieties, particularly for Me¹, are methyl, ethyl, propyl, butyl, and pentyl, of which methyl (—CH₃) is most preferred.
ar is a 5-10-membered aryl or heteroaryl moiety that is optionally substituted with 1 or 2 alkyl, halogen, or alkoxy moieties; ar is preferably a 5-10 membered heteroaryl moiety, more preferably a 5-membered heteroaryl such as thiophenyl, furanyl, or pyrrolyl, of which thiophenyl is most preferred.
X is S, NH, or O; in some embodiments it is S or O; in some embodiments it is S or NH; preferably
X is S; and
R is a C₁₋₂₅hydrocarbon moiety that can comprise 0 to 8 heteroatoms and 0 to 3 cyclic moieties.

Preferably R is -(L)m-(Cyc)n-Y; wherein m is 0, 1, or 2, preferably 0 or 1; in some embodiments m is 0; in some embodiments m is 1;
n is 0, 1, or 2, preferably 0 or 1; in some embodiments n is 0; in some embodiments n is 1; L is a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N;
Cyc is a 5 to 10 membered cyclic, heterocyclic, aromatic, or heteroaromatic moiety that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N; and
Y is H or a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N.

In one embodiment, an inhibitor of the invention is a compound of the general formula (II):

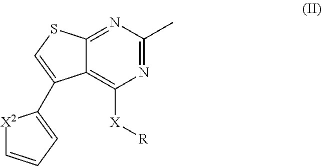

wherein X² is S, NH, or O, and wherein X and R are as defined for general formula (I) above.

In one embodiment, an inhibitor of the invention is a compound of the general formula (III):

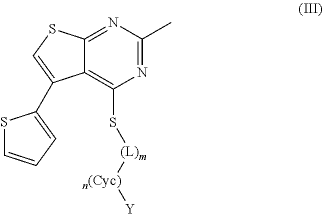

wherein
m is 0, 1, or 2, preferably 0 or 1;
n is 0, 1, or 2, preferably 0 or 1;
L is a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N;
Cyc is a 5 to 10 membered cyclic, heterocyclic, aromatic, or heteroaromatic moiety that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N; and
Y is H or a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N.

In preferred embodiments, an inhibitor of the invention is of general formula (Ia) or (lb) or (Ic):

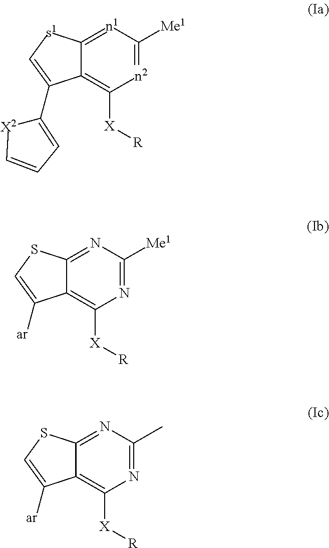

In more preferred embodiments, an inhibitor of the invention is of general formula (IIa):

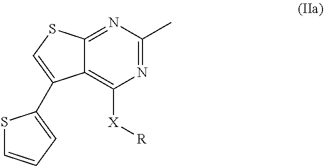

For formulas shown above, R is preferably of general formula (R1):

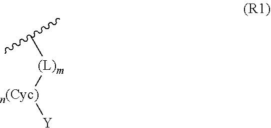

Preferred embodiments for R are as shown in Table 1 below or in Table 5 in the Examples.

TABLE 1
Suitable moieties for R with a reference number below each moiety.

| | |
|---|---|
| 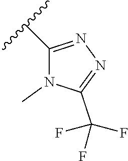 [SMILES: *c1nnc(C(F)(F)F)n1C] | R1 |
| 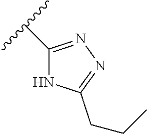 [SMILES: *c1nnc(CCC)[nH]1] | R2 |
| 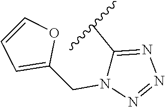 [SMILES: *c1nnnn1Cc1ccco1] | R3 |
| 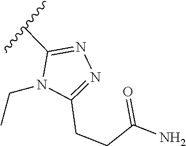 [SMILES: *c1nnc(CCC(N)=O)n1CC] | R4 |
| 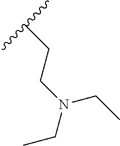 [SMILES: *CCN(CC)CC] | R5 |
| 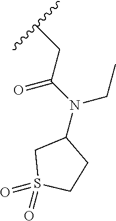 [SMILES: *CC(=O)N(CC)C1CCS(=O)(=O)C1] | R6 |
| 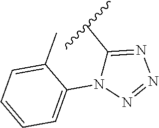 [SMILES: *c1nnnn1-c1ccccc1C] | R7 |
| 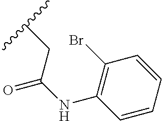 [SMILES: *CC(=O)Nc1ccccc1Br] | R8 |
| 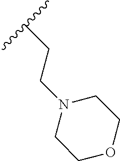 [SMILES: *CCN1CCOCC1] | R9 |
| 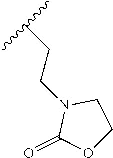 [SMILES: *CCN1CCOC1=O] | R10 |
| 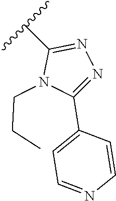 [SMILES: *c1nnc(-c2ccncc2)n1CCC] | R11 |
| 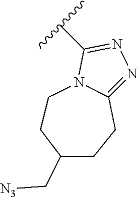 [SMILES: *c1nnc2n1CCC(CN=[N+]=[N-])CC2] | R12 |
| 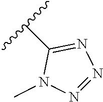 [SMILES: *c1nnnn1C] | R13 |
| 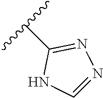 [SMILES: *c1nnc[nH]1] | R14 |
| 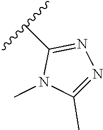 [SMILES: *c1nnc(C)n1C] | R15 |
| 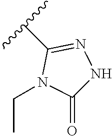 [SMILES: *c1n[nH]c(=O)n1CC] | R16 |
| 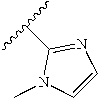 [SMILES: *c1nccn1C] | R17 |
| 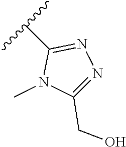 [SMILES: *c1nnc(CO)n1C] | R18 |
| 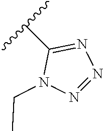 [SMILES: *c1nnnn1CC] | R19 |
| 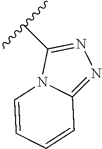 [SMILES: *c1nnc2ccccn12] | R20 |
| 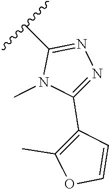 [SMILES: *c1nnc(-c2ccoc2C)n1C] | R21 |
| 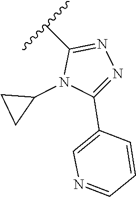 [SMILES: *c1nnc(-c2cccnc2)n1C1CC1] | R22 |
| 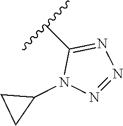 [SMILES: *c1nnnn1C1CC1] | R23 |
| 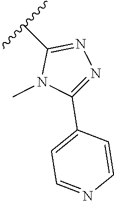 [SMILES: *c1nnc(-c2ccncc2)n1C] | R24 |
| 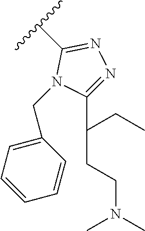 [SMILES: *c1nnc(C(CC)CCN(C)C)n1Cc1ccccc1] | R25 |
| 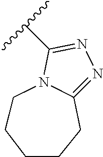 [SMILES: *c1nnc2n1CCCCC2] | R26 |
| 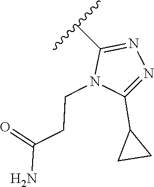 [SMILES: *c1nnc(C2CC2)n1CCC(N)=O] | R27 |
| 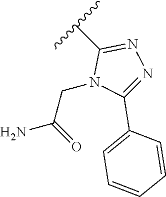 [SMILES: *c1nnc(-c2ccccc2)n1CC(N)=O] | R28 |
| 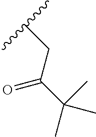 [SMILES: *CC(=O)C(C)(C)C] | R29 |
| 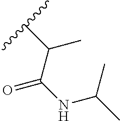 [SMILES: *C(C)C(=O)NC(C)C] | R30 |
| 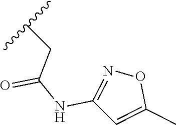 [SMILES: *CC(=O)Nc1cc(C)on1] | R31 |
| 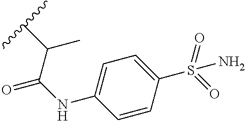 [SMILES: *C(C)C(=O)Nc1ccc(S(N)(=O)=O)cc1] | R32 |
| 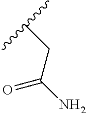 [SMILES: *CC(N)=O] | R33 |
| 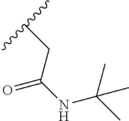 [SMILES: *CC(=O)NC(C)(C)C] | R34 |
| 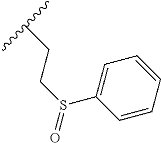 [SMILES: *CCS(=O)c1ccccc1] | R35 |
| 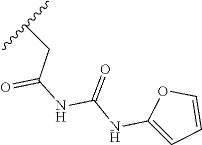 [SMILES: *CC(=O)NC(=O)Nc1ccco1] | R36 |
| 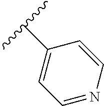 [SMILES: *c1ccncc1] | R37 |
| 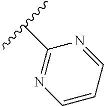 [SMILES: *c1ncccn1] | R38 |
| 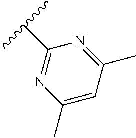 [SMILES: *c1nc(C)cc(C)n1] | R39 |
| 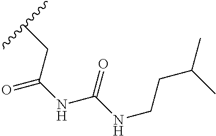 [SMILES: *CC(=O)NC(=O)NCCC(C)C] | R40 |
| 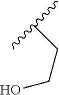 [SMILES: *CCO] | R41 |
| 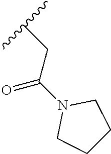 [SMILES: *CC(=O)N1CCCC1] | R42 |
| 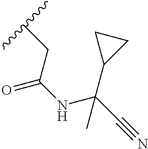 [SMILES: *CC(=O)NC(C)(C#N)C1CC1] | R43 |
| 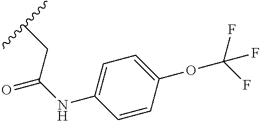 [SMILES: *CC(=O)Nc1ccc(OC(F)(F)F)cc1] | R44 |
| 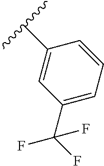 [SMILES: *c1cccc(C(F)(F)F)c1] | R45 |
| 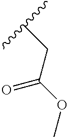 [SMILES: *CC(=O)OC] | R46 |
| 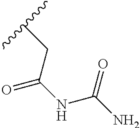 [SMILES: *CC(=O)NC(N)=O] | R47 |
| 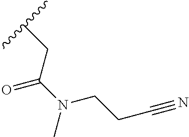 [SMILES: *CC(=O)N(C)CCC#N] | R48 |
| 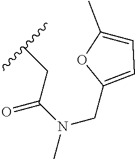 [SMILES: *CC(=O)N(C)Cc1ccc(C)o1] | R49 |
| 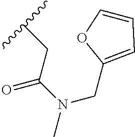 [SMILES: *CC(=O)N(C)Cc1ccco1] | R50 |
| 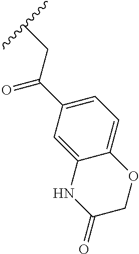 [SMILES: *CC(=O)c1ccc2c(c1)NC(=O)CO2] | R51 |
| 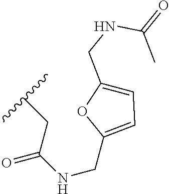 [SMILES: *CC(=O)NCc1ccc(CNC(C)=O)o1] | R52 |
| 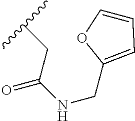 [SMILES: *CC(=O)NCc1ccco1] | R53 |
| 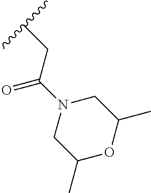 [SMILES: *CC(=O)N1CC(C)OC(C)C1] | R54 |
| 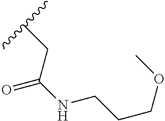 [SMILES: *CC(=O)NCCCOC] | R55 |
| 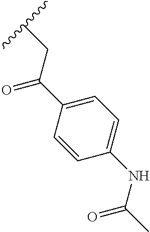 [SMILES: *CC(=O)c1ccc(NC(C)=O)cc1] | R56 |
| 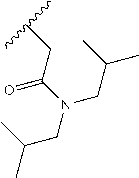 [SMILES: *CC(=O)N(CC(C)C)CC(C)C] | R57 |
| 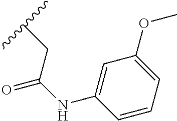 [SMILES: *CC(=O)Nc1cccc(OC)c1] | R58 |
| 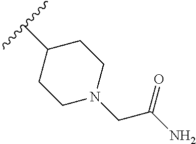 [SMILES: *C1CCN(CC(N)=O)CC1] | R59 |
| 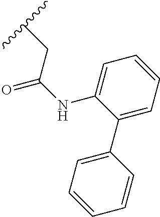 [SMILES: *CC(=O)Nc1ccccc1-c1ccccc1] | R60 |

In one embodiment, formulas shown above, R preferably comprises at least 2 non-H atoms, more preferably at least 3, even more preferably at least 4. In one embodiment, when R is an unbranched linear moiety, it preferably comprises at least five non-H atoms or at least three non-H atoms wherein a hydroxyl moiety is present. As used herein a branch is considered to be present when a carbon atom has at least one bond to more than two non-H atoms.

In one embodiment, further preferred inhibitors of the invention, if not already described above, are shown in Table 6, whereby preferably the inhibitor also meets one or more of the functional criteria for inhibitors of the invention as defined above.

### Therapeutic Use

In one embodiment, the inhibitors of the invention are used in the prevention and/or treatment of a cancer or a condition associated with or aggravated by an overactive glycolytic flux.

In one embodiment, the cancer that is prevented and/or treated using an inhibitor of the invention, is a newly diagnosed cancer that is naive to treatment, a relapsed cancer, a refractory cancer, a relapsed and refractory cancer and/or metastasis of the cancer.

In one embodiment, an inhibitor of the invention is used in the prevention and/or treatment of a cancer, wherein the cancer is a cancer or a metastasis thereof is a solid cancer/tumor. Solid tumors that may be treated with an inhibitor of the invention include, but are not limited to, adrenal cancers, bladder cancers, bone cancers, brain cancers, breast cancers (e.g., triple negative breast cancer), cervical cancers, colorectal cancers, endometrial cancers, esophageal cancers, eye cancers, gastric cancers, head and neck cancers, kidney cancers (e.g., advanced renal cell carcinoma), liver cancers (e.g., hepatocellular carcinoma, cholangiocarcinoma), lung cancers (e.g., non-small cell lung cancer, mesothelioma, small cell lung cancer), head and neck cancers, melanomas (e.g., unresectable or metastatic melanoma, advanced malignant melanoma), oral cancers, ovarian cancers, penile cancers, prostate cancers, pancreatic cancers, skin cancers (e.g., Merkel cell carcinoma), testicular cancers, thyroid cancers, uterine cancers, vaginal cancers, and tumors with evidence of DNA mismatch repair deficiency. The cancer may be newly diagnosed and naive to treatment, or may be relapsed, refractory, or relapsed and refractory, or a metastatic form of a solid tumor. In some embodiments, the solid tumor is selected from bladder cancer, breast cancer, head and neck cancer, kidney cancer, lung cancer, lymphoma, melanoma, and gastric cancer. In some embodiments, the solid tumor is selected from: melanoma (e.g., unresectable or metastatic melanoma), lung cancer (e.g., non-small cell lung cancer), and renal cell carcinoma (e.g., advanced renal cell carcinoma). In some embodiments, the solid tumor is selected from triple negative breast cancer, ovarian cancer, hepatocellular carcinoma, gastric cancer, small cell lung cancer, mesothelioma, cholangiocarcinoma, Merkel cell carcinoma and tumors with evidence of DNA mismatch repair deficiency.

In some embodiments of the invention, an inhibitor of the invention is used in the prevention and/or treatment of cancer, wherein the cancer is a blood malignancy. The blood malignancy may be newly diagnosed and naive to treatment, or may be relapsed, refractory, or relapsed and refractory, or a metastatic form of a blood malignancy. Blood-borne malignancies that may be treated with an inhibitor of the invention include, but are not limited to, myelomas (e.g., multiple myeloma), lymphomas (e.g., Hodgkin's lymphoma, non-Hodgkin's lymphoma, Waldenstrom's macroglobulinemia, mantle cell lymphoma), leukemias (e.g., chronic lymphocytic leukemia, acute myeloid leukemia, acute lymphocytic leukemia), and myelodysplastic syndromes.

In a further embodiment, an inhibitor of the invention is used in the prevention and/or treatment of a cancer or metastasis thereof as described above as adjunctive therapy, in combination with one or more (primary) treatments, which include one or more of: surgery; radiation therapy; chemotherapy, e.g. using platinum based drugs such as cisplatin and carboplatin; nucleoside analogues such as gemcitabine, taxanes such as paclitaxel and docetaxel; topoisomerase I inhibitors such as topotecan and irinotecan; topoisomerase II inhibitors such as etoposide; and anti-mitotic drugs such as vinorelbine; ‘targeted therapy’, e.g. using EGFR inhibitors such as Gefitinib; tyrosine kinase inhibitors such as Erlotinib; VEGF-A inhibitors such as bevacizumab; cyclo-oxygenase-2 inhibitors; inhibitors of cyclic guanosine monophosphate phosphodiesterase such as exisulind; proteasome inhibitors; RXR agonists such as bexarotene; and EGFR inhibitors such as cetuximab; and immunotherapy, using e.g. an immune checkpoint inhibitor, such as e.g. an antibody against PD1, PDL1, CTLA4, TIM-3 and/or LAG-3; T-cell transfer therapy, such as tumor-infiltrating lymphocytes (or TIL) therapy or CAR T-cell therapy; an antibody targeting selected TNF receptor family members, such as e.g. an antibody against CD40, 4-1 BB, CD137, OX-40/CD134 and/or CD27; an immunosuppressive cytokine such as e.g. IL-10, TGF-β and/or IL-6; and/or a γC cytokine such as e.g. IL-7, IL-15, and IL-21 and/r IL-2.

In one embodiment, an inhibitor of the invention is used in the prevention and/or treatment of a disease or condition associated with or aggravated by an overactive glycolytic flux. In addition to cancers, the Warburg effect has also been described to play a crucial role in a variety of non-tumor diseases as reviewed by Chen et al. (2018, J Cell Physiol 233(4):2839-2849). For instance, inhibition of Warburg effect can alleviate pulmonary vascular remodelling in the process of pulmonary hypertension. Interference of Warburg effect improves mitochondrial function and cardiac function in the process of cardiac hypertrophy and heart failure. Additionally, the Warburg effect induces vascular smooth muscle cell proliferation and contributes to atherosclerosis. Warburg effect may also involve in axonal damage and neuronal death, which are related with multiple sclerosis. Furthermore, Warburg effect significantly promotes cell proliferation and cyst expansion in polycystic kidney disease. Besides, Warburg effect relieves amyloid β-mediated cell death in Alzheimer's disease. And Warburg effect also improves Mycobacterium tuberculosis infection. Zhang et al (2018, Semin Nephrol 38(2):111-120) highlight the role of the Warburg effect in diabetic kidney disease, the leading cause of morbidity and mortality in diabetic patients. More recently, Palsson-McDermott and O'Neill (2013, Bioessays 35(11):965-73) and Kornberg (2020, Wiley Interdiscip Rev Syst Biol Med 12(5):e1486.doi: 10.1002/wsbm.1486) have described that pro-inflammatory signals induce metabolic reprogramming in innate and adaptive immune cells of both myeloid and lymphoid lineage, characterized by a shift to aerobic glycolysis, i.e. the Warburg effect. According to Kornberg (2020, supra), blocking this switch to aerobic glycolysis impairs the survival, differentiation, and effector functions of pro-inflammatory cell types while favouring anti-inflammatory and regulatory phenotypes, thereby providing a therapeutic opportunity to modulate immune responses in autoimmune disease without broad toxicity in other tissues of the body.

In one embodiment, therefore, the condition associated with or aggravated by an overactive glycolytic flux to be prevented and/or treated with an inhibitor of the invention is a condition or disease selected from the group consisting of: pulmonary hypertension, cardiac hypertrophy, heart failure, atherosclerosis, Alzheimer's diseases, multiple sclerosis, polycystic kidney disease, tuberculosis, diabetic kidney disease and an autoimmune disease. In one embodiment, the autoimmune disease is selected from the group consisting of: acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barre syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; warm autoimmune hemolytic anemia; and Wegener's granulomatosis.

### A Pharmaceutical Composition

In a further aspect the invention relates to a pharmaceutical composition comprising an inhibitor of the invention. Preferably, the pharmaceutical composition comprises at least one pharmaceutically acceptable carrier, in addition to the inhibitor of the invention. The pharmaceutically acceptable carrier can be any pharmaceutically acceptable carrier, adjuvant, or vehicle, that is suitable for administration to a subject. The pharmaceutical composition can be used in the methods of treatment described herein below by administration of an effective amount of the composition to a subject in need thereof. The term “subject” is used interchangeably with the term “recipient” herein, and as used herein, refers to all animals classified as mammals and includes, but is not restricted to, primates and humans. The subject is preferably a male or female human of any age or race. The treatment of the patient includes treatment in the first line or second line, or third line.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g. “Handbook of Pharmaceutical Excipients”, Rowe et al eds. 7^{th} edition, 2012, www.pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride: hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, PLURONICS™ or polyethylene glycol (PEG).

In one embodiment, the inhibitor of the invention is prepared with carriers that will protect said compound against rapid elimination from the body, such as a controlled release formulation, including implants and microencapsulated delivery systems, e.g. liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811 or US 2011305751, incorporated herein by reference.

The administration route of the inhibitor of the invention can be oral, parenteral, by inhalation or topical. The term “parenteral” as used herein includes intravenous, intra-arterial, intralymphatic, intraperitoneal, intramuscular, subcutaneous, rectal or vaginal administration. The intravenous forms of parenteral administration are preferred. By “systemic administration” is meant oral, intravenous, intraperitoneal and intramuscular administration. The amount of an inhibitor of the invention required for therapeutic or prophylactic effect will, of course, vary with the chosen, the nature and severity of the condition being treated and the patient. In addition, the inhibitor of the invention may suitably be administered by pulse infusion, e.g., with declining doses of the inhibitor. Preferably the dosing is given by injections, most preferably intravenous or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminum monostearate and gelatin.

Sterile injectable solutions can be prepared by incorporating the active compound (e.g., an inhibitor of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV) or subcutaneous (SC). Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, www.pharmrress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (an inhibitor of the invention) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, an effective administered amount of an inhibitor of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.001 to 1,000 mg/kg body weight/day, preferably about 0.01 to about 100 mg/kg body weight/day, most preferably from about 0.05 to 10 mg/kg body weight/day. More specifically, for use in accordance with the invention, the inhibitors of the invention are preferably administered at a dosage of 1-1000, 2-500, 5-200, 10-100, 20-50 or 25-35 mg/kg body weight/day, preferably administered in doses every 1, 2, 4, 7, 14 or 28 days.

The pharmaceutical compositions can be included in a container, pack, or dispenser together with instructions for administration.

The inhibitor of the invention and pharmaceutical compositions of this invention may be used with other drugs to provide a combination therapy. The other drugs may form part of the same composition or be provided as a separate composition for administration at the same time or at different time.

The present invention has been described above with reference to a number of exemplary embodiments as shown in the drawings. Modifications and alternative implementations of some parts or elements are possible, and are included in the scope of protection as defined in the appended claims.

### Examples

### Example 1

### 1.1. Results

### 1.1.1 Reduction of Glucose Uptake Restores Growth of the Tps1Δ Strain on Glucose

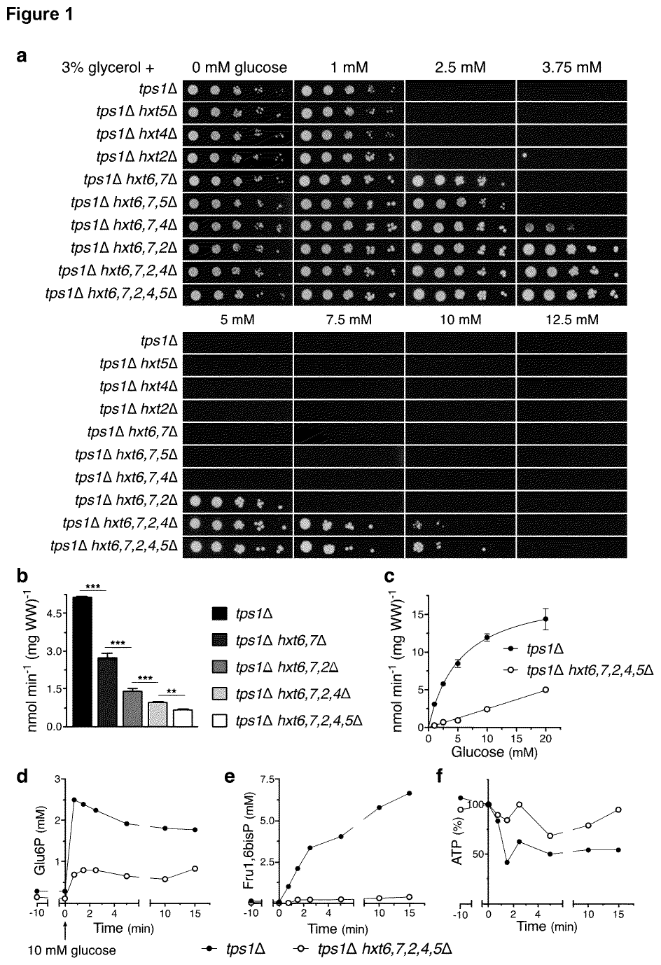

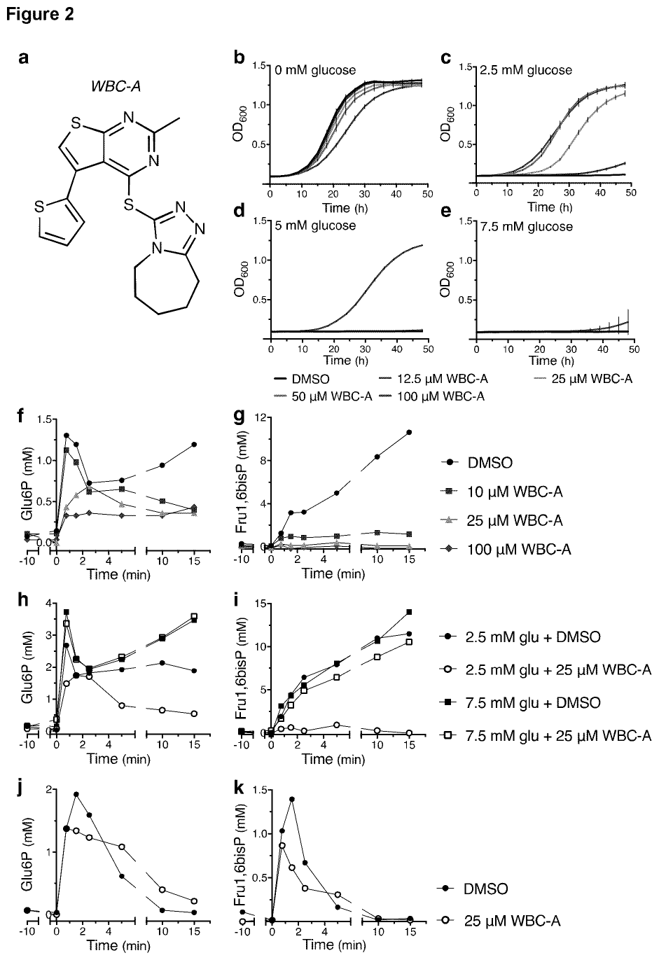

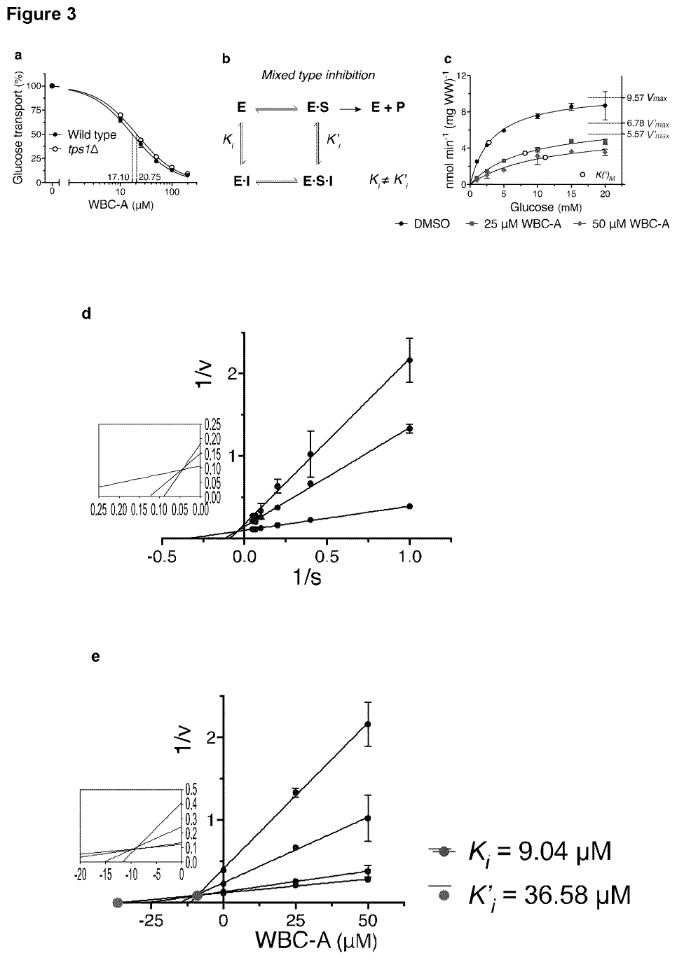

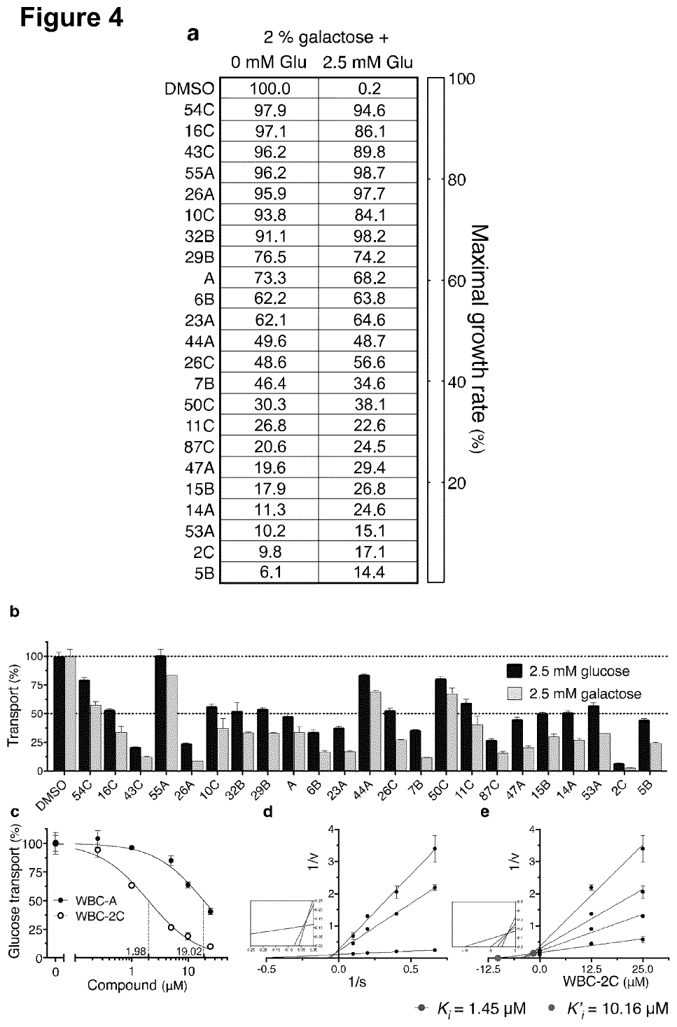

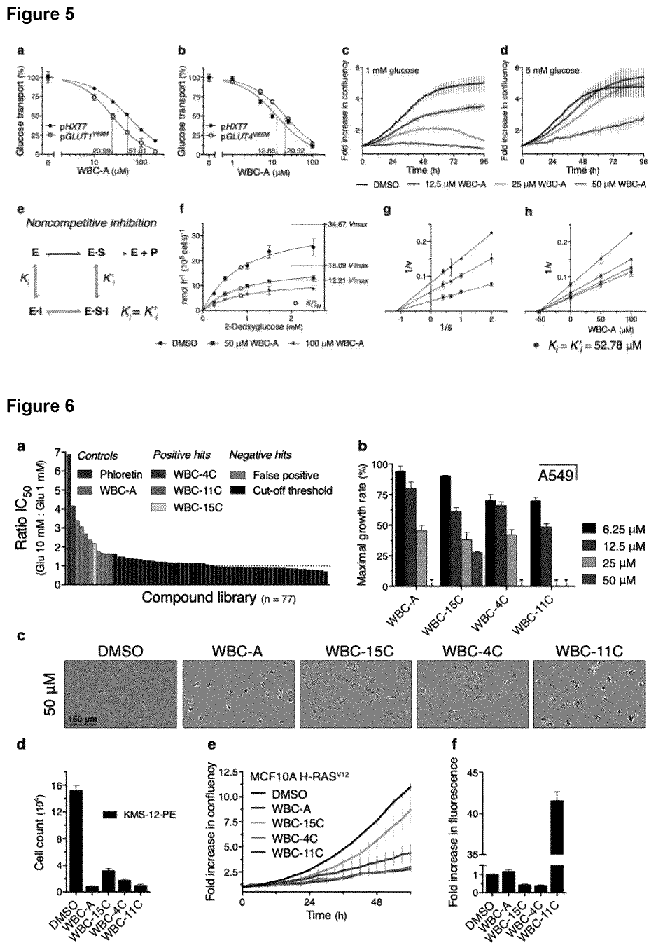

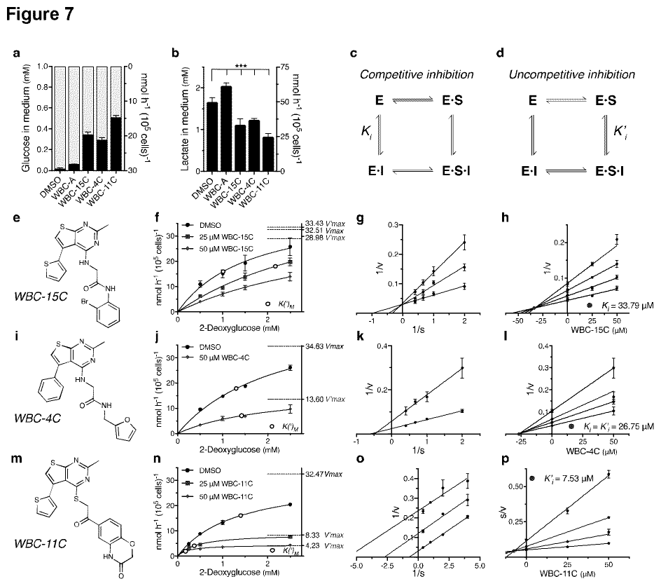

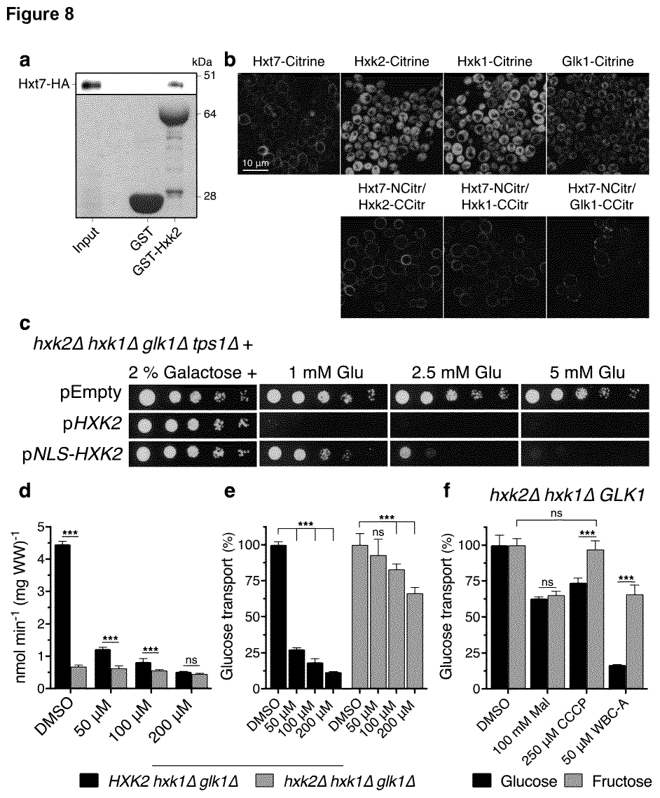

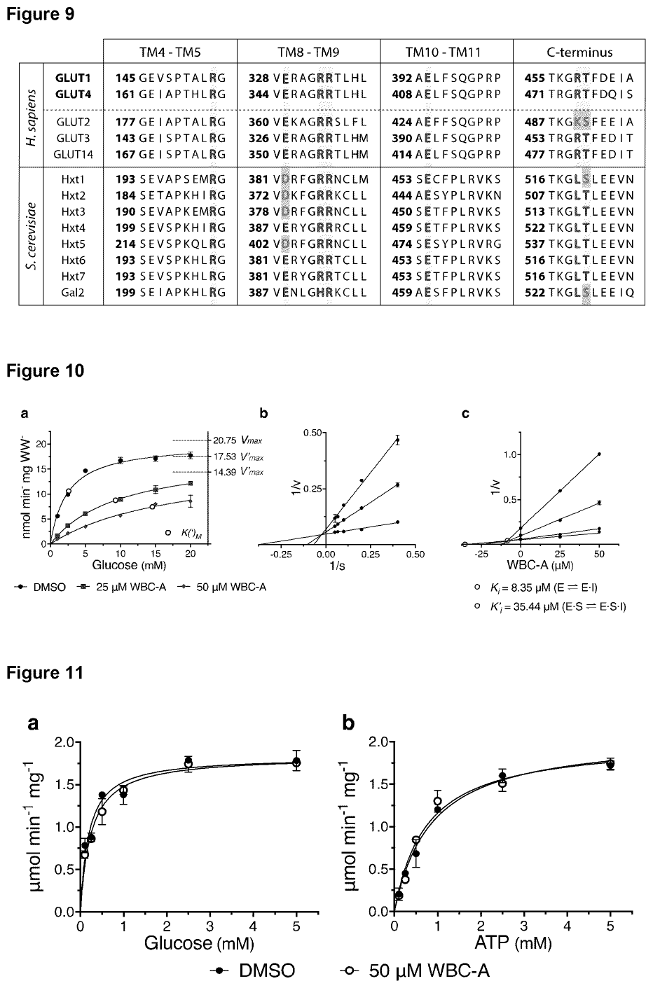

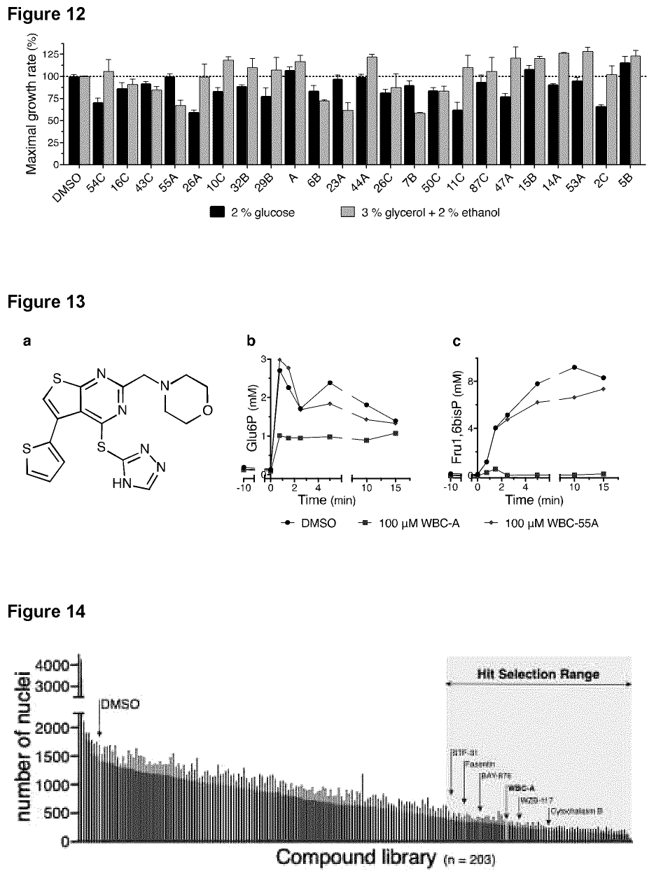

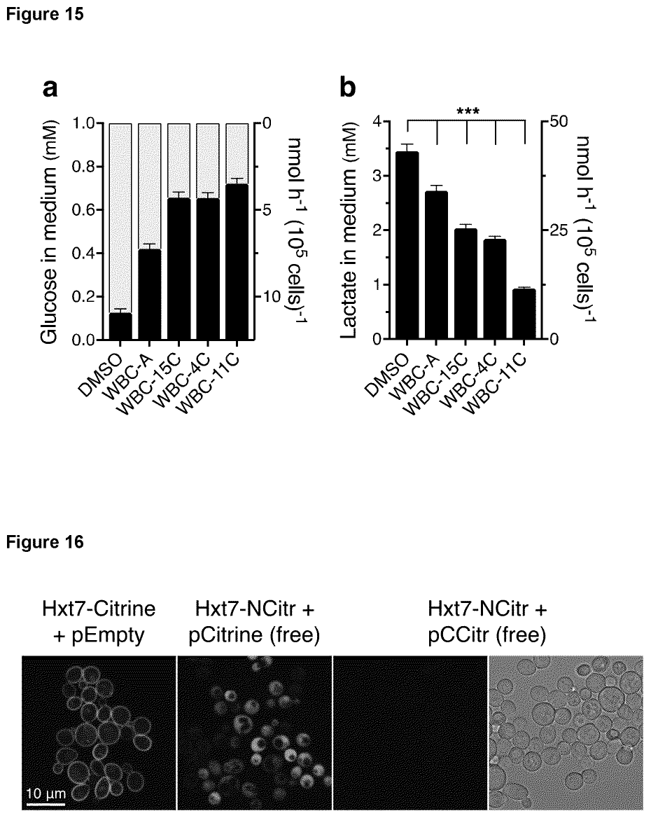

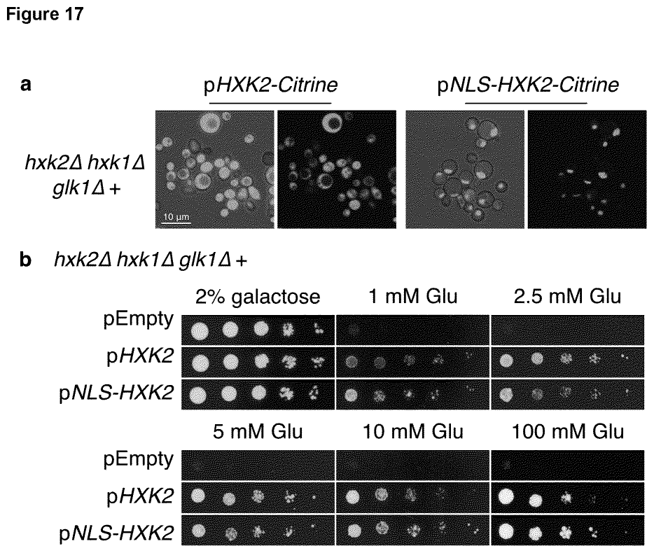

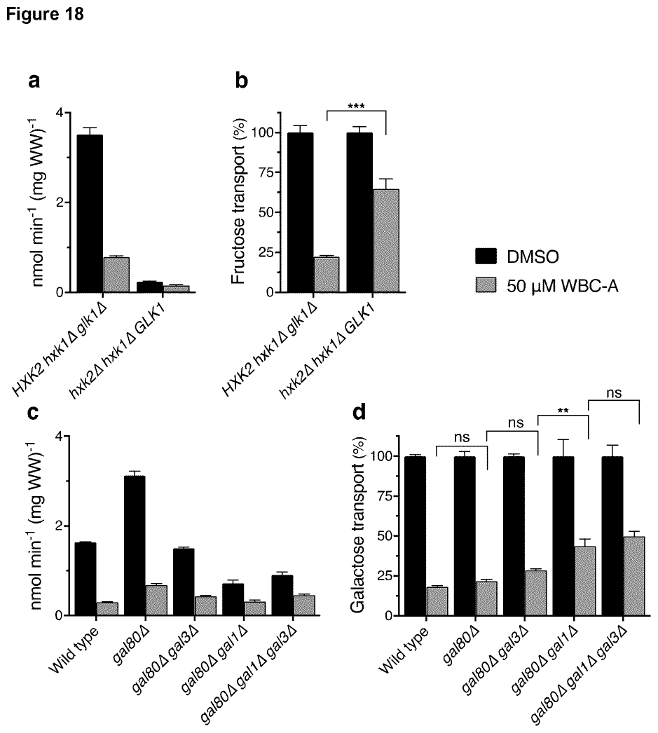

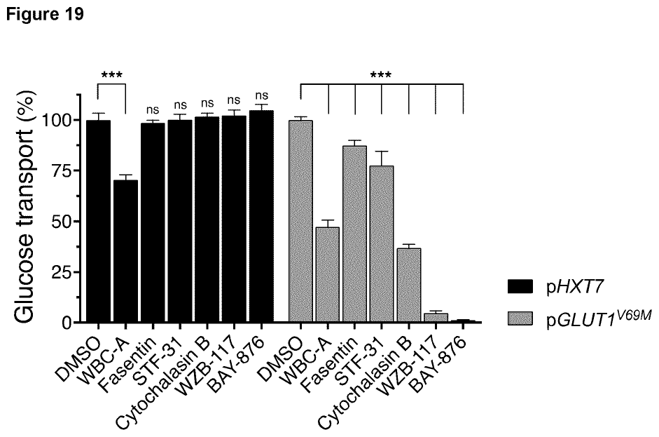

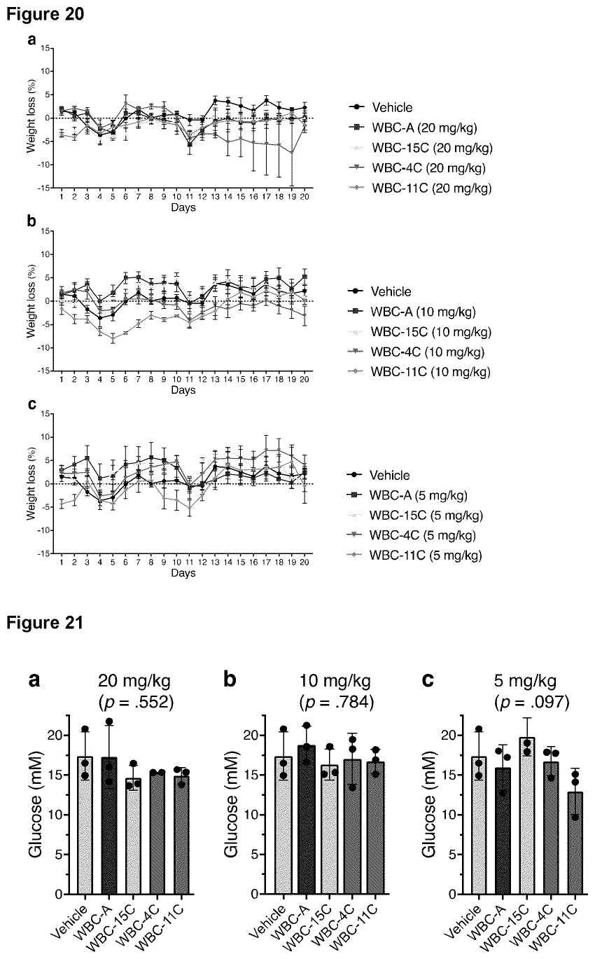

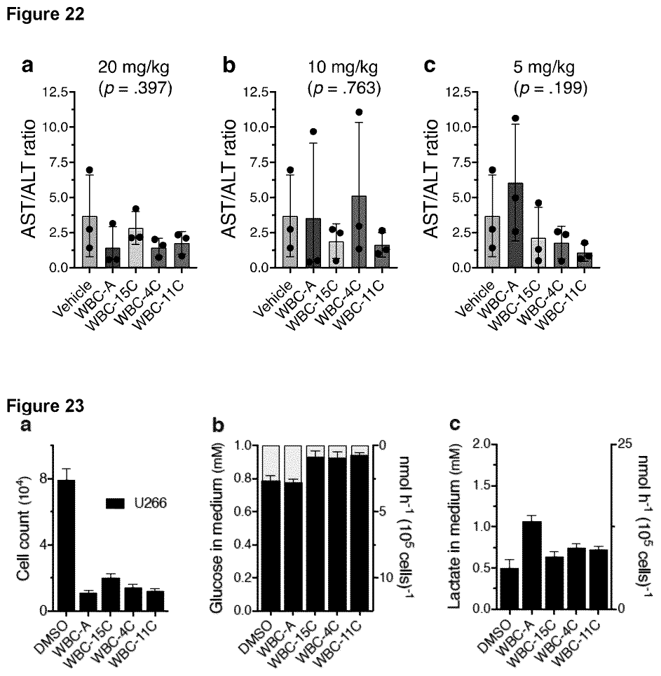

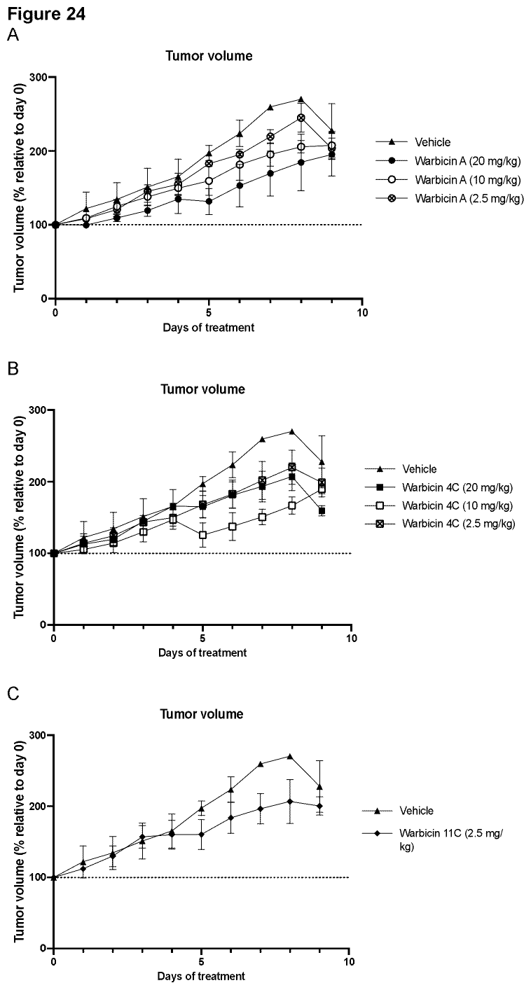

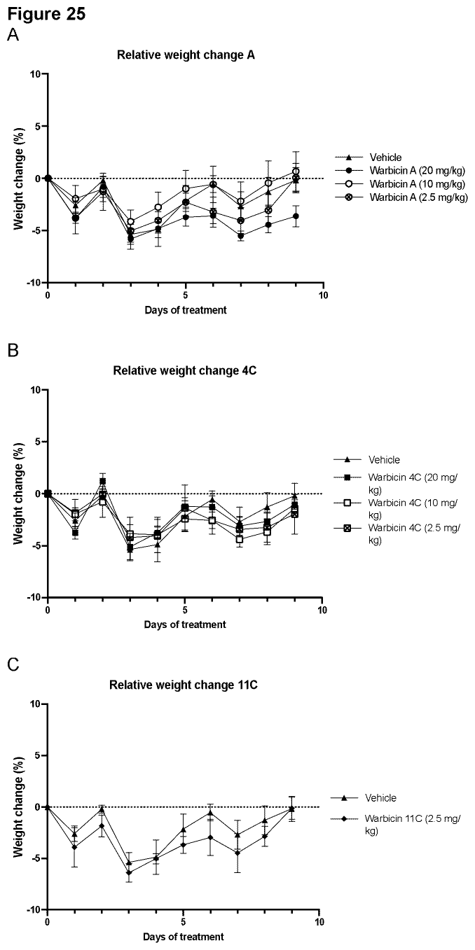

We have introduced deletions of HXT glucose carrier genes in the tps1Δ strain and assessed the effect on its glucose sensitivity. The strains were spotted in serial dilutions on solid nutrient plates containing the nonfermentable carbon source glycerol (3%) supplemented with either no glucose or increasing concentrations of glucose, from 1 to 12.5 mM (FIG. 1a). The results show that individual deletion of the HXT5, HXT4 and HXT2 genes, encoding intermediate-affinity glucose carriers is unable to restore growth on 2.5 mM glucose. On the other hand, deletion of the HXT6 and HXT7 genes, encoding the main high-affinity glucose carriers, and additional deletion of HXT5, HXT4 and/or HXT2 causes a progressive restoration of growth in the presence of increasing glucose concentrations, with additional deletion of HXT2 having the strongest effect. These results show that inactivation of glucose carrier genes is able to lower the sensitivity of the tps1Δ mutant to glucose. Direct measurements of radioactive glucose uptake in zero trans-influx experiments (2.5 mM for 10 s) confirmed that the consecutive deletion of the glucose carrier genes gradually further reduced glucose uptake capacity (FIG. 1b). The relative reduction of glucose uptake in the tps1Δ hxt6, 7, 2, 4, 5Δ strain compared to the tps1Δ strain was most pronounced at low glucose concentrations (FIG. 1c), explaining the inability of the tps1Δ hxt6, 7, 2, 4, 5Δ strain to grow at glucose concentrations higher than 10 mM (FIG. 1a). The strong reduction of glucose uptake in the tps1Δ hxt6, 7, 2, 4, 5Δ strain was correlated with lower levels of Glu6P and Fru1,6bisP, and higher levels of ATP after addition of 10 mM glucose (FIG. 1d-f). Especially the very strong reduction in Fru1,6bisP was striking.

### 1.1.2 Small-Molecule Screening for Restoration of Growth on Glucose in the Tps1Δ Strain

We have screened 40,000 small molecule compounds at the VIB core service screening facility for restoration of growth of the tps1Δ strain in the presence of 5 mM glucose in liquid YP medium with 2% galactose. Only one single compound with reproducible rescue capacity was isolated and later called Warbicin A (WBC-A). Its structure has a part that resembles the structure of adenosine (FIG. 2a). Addition of WBC-A in the absence of glucose caused insignificant inhibition of growth on galactose, except for the highest concentration of 100 μM which caused significant growth delay compared to the DMSO control (FIG. 2b). Only 100 μM WBC-A rescued growth on galactose in the presence of 5 mM glucose while lower concentrations rescued growth up to 2.5 mM glucose. On the other hand, 100 μM WBC-A was not able to restore growth in the presence of 7.5 mM glucose. Determination of the level of Glu6P and Fru1,6bisP as a function of time after addition of 2.5 mM glucose to tps1Δ cells in medium with 2% galactose showed that addition of different concentrations of WBC-A caused a concentration-dependent gradual drop in Glu6P and a more precipitous drop in Fru1,6bisP (FIG. 2f, g). The drop was dependent on the concentration of glucose. When 7.5 mM glucose was added, the presence of 25 μM WBC-A had little effect on the increase in Glu6P and Frus1,6bisP, as opposed to addition of 2.5 mM glucose (FIG. 2h, i). This is consistent with the inability of WBC-A to rescue the growth of tps1Δ cells in the presence of higher glucose concentrations (FIG. 2b-e). On the other hand, addition of 25 μM WBC-A to wild type cells caused a much smaller reduction in the increase of Glu6P and Fru1,6bisP after addition of 2.5 mM glucose (FIG. 2j, k) compared to the effect in tps1Δ cells (FIG. 2f, g). This shows that the overactive glucose phosphorylation activity in tps1Δ cells was much more sensitive to WBC-A than the regular glucose phosphorylation activity in wild type cells.

### 1.1.3 Warbicin A Acts Through Inhibition of Glucose Uptake

The inhibition of sugar phosphate hyperaccumulation by WBC-A in tps1Δ cells suggested that WBC-A may act by reducing glucose uptake. We have measured the effect of different concentrations of WBC-A on uptake activity of 2.5 mM glucose by wild type and tps1Δ cells using radioactive glucose with a 10 s time scale. WBC-A reduced glucose uptake activity similarly in wild type and tps1Δ cells with an IC₅₀ of 17.10 μM and 20.75 μM, respectively (FIG. 3a). Initial glucose uptake activity also did not differ significantly. Subsequent analysis revealed a mixed type of inhibition by WBC-A, as illustrated by the graphical representation (FIG. 3b). A kinetic analysis was performed of the inhibition of glucose uptake by 25 and 50 μM WBC-A using different glucose concentrations (FIG. 3c). Lineweaver-Burk plot analysis revealed a mixed type of inhibition (FIG. 3d) and using Dixon plot analysis we determined a Kᵢ of 9.04 μM and a K′; of 36.58 μM (FIG. 3e). The same analysis was performed for glucose uptake in tps1Δ cells, which also revealed a mixed type of inhibition with very similar inhibitor constants of 8.35 μM for Kᵢ and 35.44 μM for K′; (FIG. 10). This suggested that inhibition of glucose uptake by WBC-A is independent of Tps1.

To evaluate whether WBC-A may also act through inhibition of hexokinase activity, we determined the effect of WBC-A on hexokinase activity measured in extracts of wild type cells (FIG. 11). A variable glucose concentration and fixed ATP concentration, as well as a variable ATP concentration and fixed glucose concentration were used. No significant effect of WBC-A on in vitro hexokinase activity could be detected, further supporting the conclusion that WBC-A acts through inhibition of glucose uptake.

1.1.4 Warbicin A Analogs Rescue Growth on Glucose of Tps1Δ Cells and Inhibit Glucose Uptake with Varying Potency

Next, we screened a collection of about 200 structural analogs of WBC-A in a concentration of 100 μM for the capacity to restore growth of the tps1Δ mutant in 2% galactose liquid medium containing 2.5 mM glucose (FIG. 4a). Growth on 2% galactose in the presence of DMSO was used as reference (100%) and addition of 2.5 mM glucose in the presence of only DMSO virtually eliminated growth. Several analogs showed higher potency in this assay compared to WBC-A and they also caused less inhibition of growth on 2% galactose in the absence of 2.5 mM glucose (FIG. 4a). The reverse was seen with a series of other analogs. They showed less potency in restoring growth in the presence of 2.5 mM glucose than WBC-A and reduced growth on 2% galactose in the absence of glucose more than WBC-A (FIG. 4a). It should be noted that galactose is taken up in yeast by the Gal2 galactose/glucose carrier, which is most closely related to the high-affinity Hxt6 and Hxt7 glucose carriers among the members of the Hxt glucose carrier family^{24,63}. Hence, cross-inhibition of WBCs against Hxt glucose carriers and Gal2 can certainly not be excluded. Indeed, uptake of 2.5 mM glucose and 2.5 mM galactose were inhibited in a very similar way by a series of WBCs at a concentration of 25 μM in zero trans-influx experiments (FIG. 4b). Inhibition of galactose uptake was always more pronounced compared to glucose uptake, possibly due to the single galactose carrier Gal2 versus the many Hxt glucose carriers. Kinetic analysis of the inhibition of 2.5 mM glucose uptake by wild type cells showed that WBC-2C was a much more potent inhibitor with an IC₅₀ of 1.98 μM versus 19.02 μM for WBC-A (FIG. 4c). Lineweaver-Burk plot analysis revealed a mixed type of inhibition also for WBC-2C (FIG. 4d). Dixon plot analysis showed a Kᵢ value of 1.45 μM and a K′; of 10.16 μM for inhibition by WBC-2C (FIG. 4e).

We also evaluated for general toxicity of the WBC compounds in yeast. For that purpose, we tested the effect of a relatively high concentration of 100 μM on growth of wild type yeast cells on a fermentative medium with glucose (2%) and on a respirative medium with 3% glycerol+2% ethanol (FIG. 12). The reference compound WBC-A did not cause any growth inhibition at all on the two media. In fact, none of the compounds caused more than 50% reduction in growth. Inhibition on glucose medium was always somewhat more pronounced compared to that on respirative medium, with 55A, 23A and 7B as the most conspicuous exceptions. The majority of the Warbicins even caused an apparent slight stimulation of growth compared to the DMSO control (FIG. 12). The structure of WBC-A and the 21 analogs that reduce glucose uptake is shown in Table 5, with their corresponding IC₅₀s for 2.5 mM glucose transport inhibition and the minimal rescue concentration for tps1Δ growth on 2.5 mM glucose. Strikingly, these 21 compounds share a common backbone structure with WBC-A, that is likely important for bioactivity. WBC-55Δ was the only compound with a variation in the backbone structure (FIG. 13a). Although it caused the best rescue of tps1Δ cells on 2.5 mM glucose (FIG. 4a), it did not cause any inhibition of glucose transport in short-term 10s glucose uptake (FIG. 4b), which might relate to its deviating backbone structure. In addition, WBC-55Δ caused only minimal reduction of the hyperaccumulation of Glu6P and Fru1,6bisP after addition of 2.5 mM glucose, as opposed to the much stronger effect of WBC-A (FIG. 13b,c). This suggests that a downstream target in the signaling pathway leading from the glycolytic deregulation to growth arrest and apoptosis in the tps1Δ mutant²³ is the main target of WBC-55Δ rather than the glucose uptake system.

### 1.1.5 Warbicin A Inhibits Human GLUT1 and GLUT4 as Well as Proliferation and Glucose Uptake of Cancer Cells

To assess whether WBC-A could inhibit glucose transport activity of the GLUT carriers, we expressed human GLUT1^{V69M} and GLUT4^{V85M} in a hxt⁰ gal2Δ (RE700A) and hxt⁰ erg4Δ (EBY.VW4000) strain, respectively. These strains are both deficient in glucose uptake due to absence of all active glucose transporters. For functional GLUT expression in yeast, specific mutations had to be introduced in both sequences, whereas for GLUT4, an additional ERG4 gene deletion was required^{64,65,66}. WBC-A inhibited glucose uptake of GLUT1^{V69M} expressed in yeast with an IC₅₀ of 23.99 μM versus 51.01 μM for the yeast Hxt7 carrier solely expressed in the same hxt⁰ strain (FIG. 5a). GLUT4^{V85M} expressed in the other hxt⁰ genetic background was inhibited by WBC-A with an IC₅₀ of 20.92 μM versus 12.88 μM for yeast Hxt7 expressed in the same genetic background (FIG. 5b).

Next, we tested the effect of WBC-A on the proliferation of A549 lung adenocarcinoma cells in RPMI medium with either 1 mM glucose (FIG. 5c) or 5 mM glucose (FIG. 5d). In both cases, WBC-A caused a dose-dependent inhibition of growth, which was more pronounced on 1 mM compared to 5 mM glucose medium. In 1 mM glucose medium, 12.5 μM WBC-A caused pronounced inhibition, while higher concentrations of WBC-A completely blocked continued cell proliferation (FIG. 5c). Subsequent kinetic analysis of WBC-A inhibition of 2-deoxyglucose uptake in A549 lung adenocarcinoma cells revealed a noncompetitive type of inhibition, as illustrated by the graphical representation (FIG. 5e). Uptake of different 2-deoxyglucose concentrations was inhibited by 50 μM and 100 μM WBC-A in a dose-dependent manner (FIG. 5f. Lineweaver-Burk plot analysis revealed a noncompetitive type of inhibition (FIG. 5g) and using Dixon plot analysis, we determined a Kᵢ(=K′;) of 52.78 μM (FIG. 5h).

### 1.1.6 Screening of Structural Analogs of WBC-A for Inhibition of Cancer Cell Proliferation

We subsequently screened 203 structural analogs of WBC-A for inhibition of A549 lung adenocarcinoma cell proliferation on 1 mM glucose (FIG. 14). We also included in this assay five known inhibitors of mammalian glucose uptake: STF-31, Fasentin, BAY-876, WZB-117 and Cytochalasin B. All compounds were tested in a concentration of 50 μM. We selected the 77 most potent compounds, encompassing the range covered by the known glucose uptake inhibitors. We next determined the ratio of the IC₅₀ values for inhibition of A549 lung adenocarcinoma cell proliferation on 10 mM and 1 mM glucose for these analogs identified in the pre-screening (FIG. 6a). The compounds WBC-4C, WBC-11C and WBC-15C were singled out for further analysis since they were the only compounds among the most potent inhibitors that displayed a reproducible effect upon repetition. The other compounds turned out to be false positives that could not be validated afterwards. The maximal growth rate of A549 lung adenocarcinoma cells was reduced in a concentration-dependent manner by the Warbicin compounds with WBC-11C and WBC-15C being most and least potent, respectively, while WBC-A and WBC-4C had similar, intermediate potency (FIG. 6b). Microscopy pictures of A549 cells revealed aberrant cell morphology to different extents in the presence of these compounds (FIG. 6c). Proliferation of the multiple myeloma KMS-12-PE cell line on 1 mM glucose was also severely inhibited by 25 μM of the four Warbicin compounds. Proliferation of an MCF10Δ breast epithelia cell line transformed with the H-RAS^{V12} allele on 1 mM glucose was inhibited by the four compounds with WBC-4C and WBC-11C showing the highest potency. In the same medium, WBC-11C also triggered strong induction of apoptosis as determined by the increase in fluorescence of the Incucyte caspase 3/7 dye that turns fluorescent by caspase cleaving during induction of apoptosis (FIG. 60. Despite the weak Warburg effect of the U266 multiple myeloma cell line, 25 μM of WBC-A, -15C, -4C or -11C also inhibit proliferation of this cell line after 4 days of growth on RPMI medium with 1 mM glucose (FIG. 23a).

### 1.1.7 Characterization of WBC-15C, -4C and -11C for the Kinetics of Glucose Uptake Inhibition

The compounds WBC-15C, -4C and -11C at 25 μM inhibited glucose consumption and lactate production in A549 lung adenocarcinoma cells with increasing efficiency in this order, while WBC-A did not cause inhibition (FIG. 7a, b). The same was observed for glucose consumption and lactate production in KMS-12-PE multiple myeloma cells, with largely similar efficiency as in the A549 cells (FIG. 15). We next characterized the kinetics of 2-deoxyglucose uptake inhibition by the Warbicin compounds in A549 lung adenocarcinoma cells. A graphical representation of competitive and uncompetitive inhibition is shown in FIGS. 7c and d, respectively. The molecular structures of WBC-15C, -4C and -11C are shown in FIGS. 7 e, i and m, respectively. The inhibition of 2-deoxyglucose uptake by 50 μM WBC-15C, -4C or -11C compared to the DMSO control is shown in FIGS. 7f, j and n, respectively. Lineweaver-Burk plot analysis revealed a different type of inhibition for the three compounds, competitive for WBC-15C (FIG. 7g), noncompetitive for WBC-4C (FIG. 7k) and uncompetitive for WBC-11C (FIG. 7o). Using Dixon plot analysis, we determined a Kᵢ of 33.79 M for WBC-15C (FIG. 7h) and a Kᵢ(=K′;) of 26.75 μM for WBC-4C (FIG. 7l) and using Cornish-Bowden plot analysis a K′; of 7.53 μM for WBC-11C (FIG. 7p).

### 1.1.8 In Vitro and In Vivo Evidence for Physical Interaction Between Yeast Hxt7 and Hxk2 and Nuclear Localization of Hxk2 Rescues Growth on Low Glucose of Tps1Δ Cells

Subsequently, we have investigated possible interaction between the yeast Hxt7 glucose carrier and the hexokinase Hxk2 (FIG. 8). Using a pulldown experiment with GST-labeled Hxk2, we could precipitate HA-labeled Hxt7, indicating in vitro physical interaction between the two proteins (FIG. 8a). Hxt7 interacts in vivo with the three sugar kinases, Hxk2, Hxk1 and Glk1, as shown by Bimolecular Fluorescence Complementation (BiFC) (FIG. 8b). Confocal microscopy shows that the Hxk1-, Hxk2- and Glk1-Citrine fusion proteins are located in the cytosol, while the Hxt7-Citrine fusion protein is located at the level of the plasma membrane. On the other hand, cells expressing the fusion proteins of the N-terminal part of Citrine with Hxt7 and fusion proteins of the C-terminal part of Citrine with either Hxk1, Hxk2 or Glk1, all display fluorescence at the level of the plasma membrane (FIG. 8b). Control experiments confirmed that Hxt7 fused to the N-terminal part of Citrine does not spontaneously assemble with the C-terminal part of Citrine expressed freely in the cytosol (FIG. 16). The BIFC results support that the sugar kinases physically interact with the Hxt7 glucose transporter in vivo. To evaluate whether physical interaction of Hxk2 with Hxt7 may play a role in the tps1Δ growth defect on glucose, we fused a Nuclear Localization Sequence (NLS) sequence to Hxk2 and expressed the fusion protein in a hxk1Δ hxk2Δ glk1Δ strain. Whereas the Hxk2-Citrine protein was only present in the cytosol, the NLS-Hxk2-Citrine protein was confined to the nucleus (FIG. 17a). The fusion of the SV40 large T-antigen NLS sequence⁶⁷ to Hxk2 and the nuclear localization of NLS-Hxk2 did not affect growth of the yeast, neither on low nor on high glucose concentrations, indicating that hexokinase activity in vivo was not compromised (FIG. 17b). We next expressed Hxk2 and NLS-Hxk2 in the hxk1Δ hxk2Δ glk1Δ tps1Δ strain. This showed that confinement of NLS-Hxk2 to the nucleus rescued to some extent the tps1Δ growth defect on low glucose concentrations (FIG. 8c). This suggests that physical interaction between hexokinase and the Hxt7 glucose carrier is at least to some extent involved in the tps1Δ growth defect on glucose. Although NLS addition clearly caused sorting of Hxk2 to the nucleus, some Hxk2 may remain in the cytosol, which could also explain why rescue of the tps1Δ strain is limited to the lower glucose concentrations.

### 1.1.9 Inhibition of Glucose Uptake by WBC-A in Yeast is Dependent on Hexokinase Activity

Next, we investigated whether the inhibition of glucose uptake by WBC-A might be dependent on hexokinase activity. The glucose uptake rate in cells of the hxk1Δ hxk2Δ glk1Δ strain was reduced compared to that of the HXK2 hxk1Δ glk1Δ strain, consistent with previous literature data⁶⁸. Addition of WBC-A caused little further inhibition, while in the strain expressing active Hxk2, a strong reduction in glucose uptake rate was observed (FIG. 8d, e). These results seemed to suggest that in the absence of hexokinase activity, the carrier is much less inhibited by WBC-A and that WBC-A might target an additional process. To address this in more detail, uptake of glucose and fructose was measured in hxk2Δ hxk1Δ GLK1 cells (FIG. 8f. Here, hexose uptake was measured at tracer concentration to minimize the backflow of hexose sugar that sets in when no functional hexokinase activity is present. Reduction of the cellular ATP level with 250 μM of the protonophore CCCP reduced the glucose uptake rate but not that of fructose, which is in line with fructose not being phosphorylated by Glk1. The addition of maltose, a competitive inhibitor of glucose and fructose uptake, caused a similar reduction in the uptake for the two sugars (FIG. 80. WBC-A caused strong inhibition of glucose uptake but little inhibition of fructose uptake (FIG. 80. On the other hand, fructose transport was much more inhibited by WBC-A in a yeast strain only expressing HXK2 (>75%) compared to a strain expressing only GLK1 (±30%) (FIG. 18a, b). Although glucose is the main substrate of glucokinase, it may have some residual fructose phosphorylating activity. Also, inhibition by WBC-A of galactose uptake, which is mediated by the Gal2 galactose/glucose permease, a member of the Hxt family, was to some extent dependent on galactose phosphorylation. The latter is mainly mediated by the galactokinase Gal1²⁴. Deletion of the regulatory genes Gal80 and/or Gal3 of the Gal system, caused little reduction of WBC-A inhibitory potency (FIG. 18). These results support the importance of sugar phosphorylation for inhibition of sugar uptake by WBC-A in yeast and they support the concept that WBC-A inhibits glucose uptake by targeting in some way transport-associated phosphorylation of sugar.

1.1.10WBC-A is the Only Mammalian Glucose Uptake Inhibitor that Also Inhibits Yeast Hxt Activity

Finally, we have compared the effect of WCB-A on glucose uptake by yeast Hxt7 and human GLUT1^{V69M} with that of known mammalian glucose uptake inhibitors. For that purpose, Hxt7 and GLUT1^{V69M} were expressed in the hxt⁰ gal2Δ strain. All compounds were added at the same concentration of 25 μM. The results showed that the mammalian glucose uptake inhibitors only inhibited GLUT1^{V69M} and not Hxt7, while WBC-A was the only compound that inhibited glucose uptake both by Hxt7 and GLUT1^{V69M} (FIG. 19). These results are consistent with the notion that Warbicins inhibit glucose uptake with a different action mechanism compared to classical mammalian glucose transport inhibitors and that this mechanism is conserved between yeast Hxt and mammalian GLUT glucose carriers.

### 1.1.11 Evaluation of Warbicin Toxicity in Mice

We have evaluated the toxicity of WBC-A, WBC-15C, WBC-4C and WBC-11C in nude mice by daily intraperitoneal injection with the doses of 5, 10 and 20 mg/kg for each condition. Three mice were used for each dose. There was no significant loss in body weight over a period of 20 days, except for a small transient drop with WBC-11C at the doses of 10 and 5 mg/kg around day 5 and 11, respectively, and for one single mouse treated with 20 mg/kg WBC-4C that was sacrificed at day 19 because of 20% weight loss (FIG. 20). No obvious behavioral changes were detected and as such the mice were sacrificed at day 20. Autopsy of the sacrificed mice revealed one conspicuous deviation. Based on visual inspection only, more adipose tissue was present compared to the control mice receiving only the vehicle. This was observed with all compounds at all tested concentrations, except for a few mice belonging to the 20 (one mouse), 10 (one mouse) and 5 mg/kg (two mice) conditions of WBC-4C, respectively, and one mouse treated with 5 mg/kg WBC-11C. At the highest concentrations of WBC-15C and WBC-11C, adipose tissue was also observed in between the intestines. No other abnormalities were observed in mice treated with WBC-A or WBC-4C. At 20 mg/kg WBC-15C and at 20 and 10 mg/kg WBC-11C, toxic effects were seen in the liver and other organs (i.e. enlarged liver and intestines, white spots on the liver and fluid in the abdomen) which was not observed at the lower concentrations of these compounds tested. No significant difference could be detected in the blood glucose level compared to the control in sera samples collected post-mortem (FIG. 21). Moreover, determination of the AST/ALT ratio from the same samples, to assess liver toxicity, did not reveal any significant deviation from the control (FIG. 22).

### 1.2. Discussion

The goal of this work was to identify small molecules that would inhibit the overactive glucose uptake in cancer cells without compromising basal glucose uptake in healthy cells. Since previous work on the glucose growth defect of the yeast tps1Δ mutant revealed the conserved mechanism of Fru1,6bisP stimulation of the Ras proteins in yeast and human cells²³, we reasoned that the overactive glucose influx into yeast tps1Δ cells and human cancer cells may also be due to a conserved mechanism. While cancer cells show higher flux and higher levels of sugar phosphates in glycolysis, they maintain feedback inhibition of hexokinase by Glu6P¹⁰. On the other hand, yeast tps1Δ cells lack Tre6P for feedback inhibition of hexokinase activity³⁹. As a result, these cells accumulate sugar phosphates, and especially Fru1,6bisP, to such high levels that glycolysis rapidly becomes deregulated because of loss of ATP and Pi, persistent intracellular acidification, and apparent blockage at the level of GAPDH, ultimately triggering apoptosis and cell death. The extreme sensitivity of tps1Δ cells to glucose concentrations of only 1-2 mM in the presence of 100 mM galactose has been very puzzling. Such low glucose concentrations do not even cause glucose repression and should therefore simply be metabolized by respiration without causing any overflow metabolism at the level of pyruvate, normally leading to ethanol production. Deletion of HXK2 completely eliminates the glucose sensitivity, even to high glucose concentrations, while there is little difference in apparent total glucose phosphorylating activity, since accumulation of sugar phosphates is similar to that in wild type yeast cells⁴³. This suggests that hexokinase activity plays an unanticipated role in the high sensitivity of tps1Δ cells to glucose.

We decided to screen for small molecules that restored growth of tps1Δ cells on low levels of glucose. Hence, they should counteract hyperactive glucose influx to overcome the glucose sensitivity of tps1Δ cells, without inhibiting basal glucose influx, which is required for growth on low glucose. A single molecule, WBC-A (FIG. 2a), was isolated using a low glucose concentration in the screen. Increasing concentrations of WBC-A up to 50 μM, dose-dependently restored growth of the tps1Δ strain on 2.5 mM glucose (FIG. 2c). With 100 μM WBC-A there was no further improvement, likely due to the compromise between inhibiting overactive glucose uptake while maintaining basal glucose uptake. Also, in the presence of 5 mM glucose, 100 μM WBC-A was apparently able to reduce glucose influx enough to restore growth while still maintaining enough basal glucose uptake activity (FIG. 2d). Hence, these results confirm that WBC-A is able to reduce hyperactive glucose influx without abolishing basal glucose uptake. Higher concentrations of WBC-A showed higher potency in reducing hyperactive glucose influx in tps1Δ cells (FIG. 2f, g; FIG. 3a). Similarly, higher concentrations of WBC-A, as well as the three analogs tested in detail, caused stronger inhibition of the growth of A549 adenocarcinoma cancer cells although the concentration dependency differed among the compounds (FIG. 6b). While WBC-15C showed similar growth inhibition at the lower concentrations, it failed to cause complete inhibition at the highest concentration. This suggests that WBC-15C may affect hyperactive glucose uptake more than basal glucose uptake. Further screening of WBC analogs may reveal more compounds with a similar or even more pronounced discrepancy between the effect on hyperactive and basal glucose uptake.

In principle, we could have isolated inhibitors from our library of the downstream signaling pathway that link the glycolytic deregulation to the induction of apoptosis, but analysis of sugar phosphate accumulation in tps1Δ cells in the presence of WBC-A showed a dramatic reduction (FIG. 2f-i), clearly pointing to an upstream target for WBC-A at the level of glucose influx into glycolysis. Further analysis with 10 s zero trans-influx glucose uptake experiments showed that WBC-A caused direct inhibition of glucose uptake, which was also observed with a range of structural analogs that also restored growth of tps1Δ cells on low levels of glucose (FIG. 3a,c; FIG. 4b). This result was consistent with our demonstration that successive deletion of glucose carrier genes, especially those encoding the high-affinity transporters Hxt6 and Hxt7, in the tps1Δ strain caused a gradual restoration of growth on increasingly higher glucose levels (FIG. 1a). Galactose uptake was also inhibited to different extents by the WBC analogs (FIG. 4b) and the most potent inhibitor of galactose and glucose uptake, WBC-2C (FIG. 4c,e), restored growth in the presence of low glucose and 2% galactose in tps1Δ cells only poorly (FIG. 4a), likely because basal uptake of both sugars is essential for growth in this case. In addition, the mammalian GLUT1 and GLUT4 carriers expressed in yeast were inhibited by WBC-A (FIG. 5a,b), indicating a broad action of this type of compound on the glucose transporters of the MFS family.

The action mechanism of WCB-A, and likely that of its structural analogs, appears to be different from that of other known mammalian glucose uptake inhibitors tested in our study (FIG. 19). None of the latter inhibit glucose uptake by the yeast Hxt7 carrier. The dependency of WBC-A inhibition of glucose uptake in yeast on intracellular phosphorylation is highly unexpected and apparently unique for any glucose transport inhibitor identified up to now. It was similar for fructose but weaker for galactose, of which the transport is more than 50% slower than that of glucose and fructose. It suggests that a feature dependent on the interaction between the glucose carrier and a glucose phosphorylating enzyme may be the target of the Warbicins. Transport-associated or vectorial phosphorylation of glucose may be such a feature. It has the potential to enhance the efficiency of glucose influx into glycolysis by minimizing backflux of glucose through the glucose carrier and enabling direct access of hexokinase to incoming glucose. Suggestive evidence for the occurrence of transport-associated phosphorylation has been provided previously both in yeast^{46,47,48,49,50,51} and in mammalian cells 54, It has remained controversial, possibly because it is dynamic in nature rather than permanent as in the bacterial PTS system 45. In this work we provide evidence for a physical interaction, both in vitro and in vivo, of yeast Hxt7 and Hxk2, further supporting the possible occurrence of transport-associated phosphorylation in yeast. A dynamic nature of transport-associated phosphorylation of glucose might serve to adjust glucose influx into glycolysis according to the flux downstream in glycolysis, either being reduced when the flux and/or the ATP level is high while being enhanced when the flux and/or the ATP level are too low. It is tempting to speculate that the overactive influx of glucose into glycolysis in cancer cells and in yeast tps1Δ cells may be due to aberrant control of transport-associated phosphorylation of glucose. Since tps1Δ cells lack feedback inhibition by Tre6P on hexokinase this would lead to permanent deregulation of glycolysis and ultimately cell death. Since cancer cells, on the other hand, still have feedback inhibition by Glu6P on hexokinase, it would lead to permanently overactive but still controlled glucose influx into glycolysis, creating the Warburg effect, aerobic fermentation and stimulation of the uncontrolled cellular proliferation. Aberrant transport-associated phosphorylation of glucose can be caused by erroneous allosteric or protein regulatory control, faulty post-translational modification and/or aberrant overexpression of glucose carriers and hexokinases.

The mammalian glucose carriers GLUT1 and GLUT4 are known to have a cytosolic ATP-binding domain, in which the bound ATP molecule inhibits glucose uptake by the carrier^{29,31,69,70,71}. Non-hydrolyzable analogs of ATP cause similar inhibition of glucose uptake by GLUT1, indicating that inhibition is a direct consequence of ATP binding to GLUT1 and does not require ATP utilization as a source of energy⁷². ATP binds to a Walker B motif located at the cytoplasmic loop between TM8 and TM9 in GLUT1³² and the same ATP binding domain is present in GLUT4³⁴. ATP binding causes a constriction in the glucose transport channel, thereby lowering glucose uptake⁷². The R349, R350, R474, T475 and E409 residues in GLUT4, which are fully conserved in GLUT1, are responsible for ATP binding, which is controlled by the proton-sensitive, intracellular saltbridges, E329-R333/R334 in GLUT1 and E345-R349/R350 in GLUT4. The latter salt bridge network is proposed to switch upon ATP binding to the E345-R169-E409 salt bridge network^{30,32,33}. We show that all these residues are largely conserved in the yeast Hxt transporters (FIG. 9), making it highly likely that they share the same ATP-binding domain and similar regulation of glucose uptake by the bound ATP as the human GLUT carriers. Up to now, however, ATP binding to yeast Hxt carriers has not been experimentally verified.

The presence of a bound ATP molecule to the cytosolic domain of the glucose carriers and the evidence for glucose carrier/hexokinase interaction, suggests a novel mechanism by which interacting hexokinases might control glucose uptake by the carriers. If the bound hexokinase is able to use the carrier-bound ATP molecule as a substrate for phosphorylation of incoming glucose, hydrolysis of the ATP would abolish its inhibition of glucose uptake and thus cause increased glucose influx. ADP also binds into the ATP-binding pocket but is unable to inhibit glucose uptake^{28,73}. Hence, ADP would have to be exchanged for new ATP to reestablish inhibition of glucose uptake. Utilization of carrier-bound ATP might depend on the cytosolic ATP level in the cells, being low when cytosolic ATP is high and high when cytosolic ATP is low. In this way, glucose carrier/hexokinase interaction could adjust glucose influx into glycolysis to the flux downstream in glycolysis in order to maintain ATP homeostasis. The process suggested may be part of the transport-associated (vectorial) phosphorylation process of glucose by the interacting hexokinase, but the increase in glucose influx in glycolysis would not just be the result of the higher efficiency of direct metabolite channeling of incoming glucose from the carrier to the hexokinase 74, but also due to relief of ATP inhibition on influx. Based on this new mechanism, we suggest that aberrant interaction of hexokinase with the glucose carriers in cancer cells and tps1Δ cells might cause persistent hydrolysis of the bound ATP molecule resulting in permanent overactive influx of glucose into glycolysis.

WBC-A and nearly all its active structural analogs share a common adenine-like moiety, which appears to be essential for activity in inhibiting glucose uptake. With the single exception of WBC-55Δ (FIG. 13a), all compounds that rescued the tps1Δ strain on low glucose had a different modification of the sulphur-linked side group attached to the adenine-like structure (Table 5), while other variations completely abolished the rescue of the tps1Δ mutant on low glucose (Table 6).

WBC-55C was highly active in rescuing growth of tps1Δ cells on glucose. However, it did not prevent hyperaccumulation of sugar phosphates, at least not in the short term, suggesting that its main target might be located downstream in the signaling pathway between the glycolytic deregulation and the induction of apoptosis.

Adenosine⁷¹ and caffeine⁷⁵ also bind to GLUT1 and inhibit glucose uptake similarly to ATP⁷¹. This suggests that the Warbicins may act as partial structural analogs of ATP and bind into the same ATP-binding domain of the glucose carriers. Warbicins would thus take over the inhibition by ATP on glucose import and prevent its (overactive) hydrolysis by the interacting hexokinase. This hypothesis is supported by the large dependency of Warbicin inhibition of glucose uptake on the presence of active hexokinase in the cells. Moreover, restraining hexokinase in the nucleus lowers the glucose sensitivity of tps1Δ cells (FIG. 8c), consistent with involvement of glucose carrier/hexokinase interaction in the glucose growth defect of tps1Δ cells. The strong dependency on glucose phosphorylation of Warbicin inhibition of glucose uptake might be due to the low efficiency with which Warbicins can replace ATP bound to the glucose carrier. When interacting hexokinase hydrolyzes bound ATP with subsequent release of ADP and exchange for new ATP, the rate of Warbicin binding into the ATP-binding domain could be enhanced. Hence, hexokinase utilization of carrier-bound ATP might stimulate the exchange of ATP for Warbicin and thus explain the dependency of Warbicin inhibition on the presence of hexokinase. Since Warbicin cannot be hydrolyzed by hexokinase, its binding would cause permanent inhibition as opposed to the inhibition by ATP. Alternatively, in cells without glucose phosphorylating enzymes, ATP would be expected to be bound constitutively to the glucose carrier and its exchange for Warbicin would therefore make little difference. In both cases, the glucose carrier would be inhibited all the time and Warbicin would have little further effect.

The kinetics of Warbicin inhibition are complex as different Warbicins display different types of inhibition and high glucose concentrations overcome their inhibitory effect. This may be beneficial for cancer chemotherapy by offering a range of closely-related drugs affecting glucose influx into cancer cells with different kinetics, increasing the chances of preferentially inhibiting overactive glucose influx into cancer cells and not basal glucose influx into healthy cells. A wealth of evidence is available that cancer cells are highly dependent on the hyperactive flux through glycolysis or the Warburg effect, and that inhibition of glycolysis enhances the sensitivity of cancer cells to different types of cancer treatments, such as chemotherapeutics and radiation^{75,76,77}. In that respect, also the GLUT transporters have been proposed as attractive targets for anticancer drug treatment⁷⁸ especially since glucose uptake may have a major role in rate-control of glycolytic flux in cancer cells^{8,10}. Several reports have documented that inhibitors or other mechanisms of GLUT downregulation render cancer cells more drug-sensitive^{79,80,81,82,83,84,85}. Up to now, however, no drugs have been available that preferentially act on the hyperactive glucose flux in cancer cells without compromising glycolytic flux in healthy cells, so as to minimize any side-effects that interference with basal cellular metabolism may have. The latter is essential since virtually all cells in the body depend on glucose metabolism for maintenance of their viability and execution of their cellular functions.

Homology modeling suggested that GLUT4 exists in three different forms: substrate free (apo), glucose bound and glucose-ATP bound form³³. If the same is true for the yeast Hxt carriers, it would imply that cells pregrown on a non-fermentable carbon source have ATP-free Hxt glucose carriers with unrestricted potential for glucose import. Only when glucose is added and bound to the glucose transporter, intracellular ATP would be able to bind to the glucose carriers and restrict glucose influx. This might explain why intracellular Glu6P increases so strongly and rapidly in the first 30s after glucose addition and then suddenly declines in wild type cells or saturates in tps1Δ cells. This might be the timeframe in which ATP binds to all the glucose carriers in order to restrict glucose influx. The fact that ATP can only bind to the glucose carriers after glucose has been added, might explain why in stopped-flow subsecond glucose uptake experiments no effect could be detected of the sugar kinases on glucose uptake kinetics⁵. On this time scale, ATP might not be bound yet to the glucose carriers, and thus not able to inhibit their activity or influence their kinetics. Since in this model hexokinase is supposed to influence glucose carrier kinetics through its hydrolysis of the bound ATP molecule, measurements on the subsecond time scale would also not be able to detect an influence of the hexokinases on glucose carrier kinetics.

In conclusion, we suggest that aberrant transport-associated phosphorylation of glucose, in which ATP bound to the cytosolic domain of the glucose carriers is used as substrate by hexokinase, is responsible for persistent hydrolysis of this ATP molecule in tps1Δ yeast cells and in cancer cells, causing permanent overactive glucose influx. Warbicins are then proposed to inhibit overactive glucose influx into glycolysis by replacing ATP as a non-hydrolyzable ATP substitute and restoring to some extent the inhibition of glucose uptake. In the absence of interacting hexokinase, ATP is permanently bound to the glucose carriers and Warbicins therefore have little further effect, explaining why their inhibition of glucose uptake is dependent on glucose phosphorylation.

### 1.3. Materials and Methods

### 1.3.1 Cell Line and Plasmid Overview

(i) S. cerevisiae

All S. cerevisiae strains used and constructed in this work shared the W303 genetic background (Table 2), with the exception of the hxt⁰ strains with the RE700A and EBY.VW4000 backgrounds. Both gene deletion and plasmid transformation strategies were performed by following the Gietz heat shock protocol⁸⁶). For genomic deletions, either antibiotic resistance gene cassettes or auxotrophic markers were PCR amplified with primer tails homologous to the 50 bp upstream and downstream regions of the gene of interest. Using heat shock at 42° C. for 30 min, cells were transformed for homologous recombination or for introduction of a plasmid. For antibiotic selection, cells were incubated in medium without selection for 4 h prior to plating, whereas for auxotrophic selection, cells were plated immediately after the transformation protocol.

TABLE 2
Overview of the S. cerevisiae strains used and constructed in this work.

| | | | Source/ |
|---|---|---|---|
| Strain | Main genotype | Complete genotype | Reference |
| W303-1A | Wild type for all | MATa leu2-3, 112 trp1-1 can1-100 | ⁹² |
| | strains, except where | ura3-1 ade2-1 his3-11, 15 GAL SUC | |
| | indicated | mal | |
| WVT2 | tps1Δ | tps1::KanMX | This work |
| WVT27 | tps1Δ hxt2Δ | tps1::KanMX, hxt2::NatMX | This work |
| WVT28 | tps1Δ hxt4Δ | tps1::KanMX, hxt4::NatMX | This work |
| WVT29 | tps1Δ hxt5Δ | tps1::KanMX, | This work |
| | | hxt5::NatMX | |
| WVT22 | tps1Δ hxt6, 7Δ | tps1::KanMX, | This work |
| | | hxt6, 7::NatMX | |
| WVT24 | tps1Δ hxt6, 7, 4Δ | tps1::HphMX, hxt6, 7::NatMX, | This work |
| | | hxt4::KanMX | |
| WVT25 | tps1Δ hxt6, 7, 5Δ | tps1::HphMX, hxt6, 7::NatMX, | This work |
| | | hxt5::KanMX | |
| WVT23 | tps1Δ hxt6, 7, 2Δ | tps1::HphMX, hxt6, 7::NatMX, | This work |
| | | hxt2::KanMX | |
| WVT35 | tps1Δ hxt6, 7, 2, 4Δ | tps1::KanMX, hxt6, 7::looped, | This work |
| | | hxt2::NatMX, hxt4::looped | |
| WVT36 | tps1Δ hxt6, 7, 2, 4, 5Δ | tps1::HphMX, hxt6, 7::looped, | This work |
| | | hxt2::NatMX, hxt4::looped, | |
| | | hxt5::KanMX | |
| WVT8 | Hxt7-Citrine | hxt7::HXT7-Citrine-KanMX | This work |
| WVT9 | Hxk2-Citrine | hxk2::HXK2-Citrine-KanMX | This work |
| WVT39 | Hxk1-Citrine | hxk1::HXK1-Citrine-KanMX | This work |
| WVT40 | Glk1-Citrine | glk1::GLK1-Citrine-KanMX | This work |
| WVT41 | Hxt7-NCitr | hxt7::HXT7-NCit-URA3 | This work |
| WVT11 | Hxt7-NCitr Hxk2-CCitr | hxt7::HXT7-NCit-URA3, hxk2::HXK2- | This work |
| | | CCit-HIS3 | |
| WVT42 | Hxt7-NCitr Hxk1-CCitr | hxt7::HXT7-NCit-URA3, hxk1::HXK1- | This work |
| | | CCit-HIS3 | |
| WVT43 | Hxt7-NCitr Glk1-CCitr | hxt7::HXT7-NCit-URA3, glk1::GLK1- | This work |
| | | CCit-HIS3 | |
| WVT46 | HXK2 hxk1Δ glk1Δ | hxk1::NatMX, glk1::KanMX | This work |
| WVT47 | hxk2Δ HXK1 glk1Δ | hxk2::NatMX, glk1::KanMX | This work |
| WVT48 | hxk2Δ hxk1 GLK1 | hxk2::NatMX, hxk1::KanMX | This work |
| WVT49 | hxk2Δ hxk1Δ glk1Δ | hxk2::HphMX, hxk1::NatMX, | This work |
| | | glk1::KanMX | |
| JT20836 | hxk2Δ hxk1Δ glk1Δ | hxk2::TRP1, hxk1::HIS3, | This work |
| | tps1Δ | glk1::KanMX, tps1::TRP1 | |
| WVT50 | gal80Δ | gal80::NatMX | This work |
| WVT51 | gal80Δ gal1Δ | gal80::NatMX, gal1::KanMX | This work |
| WVT52 | gal80Δ gal3Δ | gal80::NatMX, gal3::KanMX | This work |
| WVT53 | gal80Δ gal3Δ gal1Δ | gal80::NatMX, gal3::KanMX, | This work |
| | | gal1::HphMX | |
| JT5312 | RE700Δ hxt⁰ gal2Δ | RE700A MATa hxt1, 2, 3, 4, 5, 7Δ, | This work |
| | | gal2::URA3 | |
| WVT54 | EBY.VW4000 hxt⁰ | EBY.VW4000 leu2-3, 112 ura3-52 | This work |
| | erg4Δ | trp1-289 his3Δ1 MAL2-8C SUC2 | |
| | | hxt17Δ hxt13Δ hxt15Δ hxt16Δ hxt14Δ | |
| | | hxt12Δ hxt9Δ hxt11Δ hxt10Δ hxt8Δ | |
| | | hxt1-7Δ gal2Δ sit1Δ agt14 ydl247wΔ | |
| | | yjr160cΔ, erg4::NatMX | |

### (ii) Human Cell Lines

An overview of the human cell lines used in this work is shown in Table 3. Growth analysis of adherent epithelial cell lines was performed in the laboratory of Cellular Metabolism and Metabolic Regulation. The human cell lines were also cultivated in the L2 cell culture room in the Molecular Cell Biology laboratory of Prof. Patrick Van Dijck (KU Leuven, VIB) for glucose uptake studies.

TABLE 3
Overview of the human cell lines used in this work.

| Cell line | Reference | Source |
|---|---|---|
| A549, lung adenocarcinoma (type II | ATCC, CCL-185 | Kindly provided by Prof. |
| alveolar epithelium) | | Sarah-Maria Fendt. |
| MCF10A, non-tumorigenic breast | ATCC, CRL-10317 | Kindly provided by Prof. |
| epithelia transformed with H-RAS^{V12} | (untransformed) | Sarah-Maria Fendt. |
| KMS-12-PE, multiple myeloma | DSMZ ACC-606 | Kindly provided by Prof. |
| | | Michel Delforge. |
| U266 multiple myeloma | ATCC, TIB-196 ™ | |

(iii) Plasmids

Plasmids were constructed using Gibson Assembly® cloning. Vectors of choice were always double digested and sticky ends were dephosphorylated by FastAP™. Inserts were PCR amplified by the Q5® High-Fidelity polymerase and both the digested vectors and inserts were first gel-purified. Finally, Gibson cloning was performed by adding Gibson reagent in the recommended ratio of insert to vector. After incubation at 50° C. for 30 min, the Gibson reaction mixture was transformed into TOP10 E. coli cells by heat shock. Positive clones were validated by PCR and sequence analysis. An overview of the plasmids used in this work is given in Table 4. For the cloning of HXT7 and HXK2, DNA from the W303 genetic background was used as genomic template. The full-length GLUT1 and GLUT4 coding sequences were obtained from the UniProt database and were ordered as gBlocks™ from IDTechnologies. Additional point mutations were introduced by PCR amplification, using primers with tails designed to bear the intended mutations.

TABLE 4
Overview of the plasmids used and constructed in this work.

| | Plasmid | | Selection | | |
|---|---|---|---|---|---|
| Plasmid | backbone | Vector elements | marker | Insert | Source |
| PYX112- | PYX112 | TPI1 prom., low- | URA3 | HXT7-HA | This work |
| HXT7-HA | | copy | | | |
| PYX122 | PYX122 | TPI1 prom., low- | HIS3 | Empty | MCB Lab, KU |
| (-Empty) | | copy | | | Leuven |
| PYX122- | | | | Citrine | This work |
| Citrine | | | | | |
| PYX122- | | | | CCitr | This work |
| CCitr | | | | | |
| pFL38Kan- | pFL38- | TEF1 prom., low- | KanMX | HXT7-HA | This work |
| HXT7-HA | Kan | copy | | | |
| pFL38Kan- | | | | GLUT1^{V69M}- | This work |
| GLUT1^{V69M} | | | | HA | |
| pFL38Kan- | | | | GLUT4^{V85M}- | This work |
| GLUT4^{V85M} | | | | HA | |
| PYC33 | YCpLAC33 | Low-copy | URA3 | Empty | MCB Lab, KU |
| (-Empty) | | | | | Leuven |
| PYC33- | | | | HXK2 | This work |
| HXK2 | | | | | |
| PYC33- | | | | HXK2-Citrine | This work |
| HXK2-Citrine | | | | | |
| PYC33-NLS- | | | | NLS-HXK2 | This work |
| HXK2 | | | | | |
| PYC33-NLS- | | | | NLS-HXK2- | This work |
| HXK2-Citrine | | | | Citrine | |
| pGEX-GST | pGEX4T- | Tac prom. | Amp | GST | GE Healthcare |
| pGEX-GST- | 1 | | | GST-HXK2 | This work |
| HXK2 | | | | | |

### 1.3.2 General Growth Conditions

(i) E. coli

E. coli cells were always cultivated in Luria broth (LB) medium at 37° C. For plasmid retention and propagation in TOP10 E. coli cells, 100 μg/mL ampicillin was added to liquid medium or solid plates. For protein production, BL21 E. coli cells were grown to exponential phase at 37° C. and shifted to 20° C. overnight after isopropyl R-D-1-thiogalactopyranoside induction (IPTG). Cells were made heat shock competent by rubidium chloride⁸⁷ after which vials were stored at −80° C. for up to one year.

(ii) S. cerevisiae

Cells of S. cerevisiae were either grown in minimal or rich medium. For minimal medium, cells were grown in Complete Synthetic medium (MP biomedicals) containing 0.5% (w/v) ammonium sulphate (Sigma-Aldrich), 0.17% (w/v) yeast nitrogen base without amino acids and without ammonium sulphate, supplemented with 100 mg/L adenine. In the case of auxotrophic selection, the appropriate composition of essential amino acids (MP Biomedicals) was chosen. For liquid medium, pH was adjusted to 5.5 and for solid medium (2% agar) to 6.5 with KOH. For rich medium, cells were grown on YP medium (1% w/v yeast extract, 2% w/v bacteriological peptone), supplemented with 100 mg/L adenine. S. cerevisiae cells were always grown at 30° C. Liquid cultures were shaken at 200 rpm.

(iii) Human Cell Lines

The A549 cell line was always propagated in RPMI-1640 (Gibco®) medium containing 10 mM glucose. Medium was supplemented with heat-inactivated 10% Fetal Bovine Serum (FBS) and 1% (50 units/mL) Penicillin/Streptomycin (Pen/Strep, Thermo Fisher Scientific). A549 cells were passaged close to 80-90% confluency. MCF10Δ H-RAS^{V12} cells were cultured in DMEM/F12 (Gibco®) medium supplemented with 5% horse serum, 1% Pen/Strep, 10 μg/mL insulin, 0.5 μg/mL hydrocortisone, 100 ng/mL cholera toxin and 20 ng/mL recombinant human EGF⁸⁸. MCF10Δ H-RAS^{V12} cells were always passaged before reaching 50% confluency. The KMS-12-PE cell line was cultured in RPMI medium containing 10 mM glucose, 20% heat-inactivated FBS and 1% Pen/Strep. Since these cells grow in suspension, cell density was kept between 105 and 10⁶ cells/mL. For assay purposes with varying glucose concentrations, appropriate amounts of medium containing glucose or no glucose were mixed to achieve the intended composition. Finally, all medium solutions were filter-sterilized prior to use. For passaging, cells were always washed first with dPBS (Gibco®), and for adherent cells, detached by Trypsin-EDTA (Gibco™) digestion for 5 min. Cells were always incubated at 37° C. in the presence of 5% CO₂.

### 1.3.3 Compound ID and Handling

WBC-A and the complete structural analog library (Table 6) were purchased from different vendors, i.e. Enamine (n=185), Life Chemicals (n=10), LabNetwork (n=4) and Uorsy (n=4). WBC compounds in general are very insoluble in aqueous solutions, challenging their solubilization. Throughout this work, WBC compounds were always stock aliquoted at 50 mM concentration in 100% DMSO. In many cases, gentle sonication and heating up to 37° C. was necessary to completely solubilize the compound stocks. Whereas WBC-A, -15C, -4C and -11C could be dissolved at 50 mM, some structural analogs remained in smooth or rough suspension, regardless of the concentration. Even though it is recommended to aliquot compound stocks, no significant reduction in compound strength by repeated freeze and thaw cycles in DMSO was observed.

### 1.3.4 Assessment of Cell Proliferation and Viability

### 1.3.4.1 Spot Dilution Assays

In general, glucose growth sensitivity or restoration was assessed by adding increasing amounts of glucose to the plates. For tps1Δ (hxtA) glucose sensitivity (FIG. 1a) and the restoration of hxk2Δ hxk1Δ glk1Δ glucose growth (Supplementary FIG. 8b) cells were always pregrown on Complete Synthetic medium containing 3% glycerol. For the tps1Δ (hxtA) spot assay, 3% glycerol was added in all the plates. For the assessment of hxk2Δ hxk1Δ glk1Δ tps1Δ glucose sensitivity (FIG. 8c), cells were pregrown on 2% galactose which was also added as a base carbon source in all plates. After pregrowth overnight, cells were harvested, washed with medium without sugar and resuspended to an OD of 0.25. Cells were spotted in five-fold dilution in 3 μL droplets each. Plates were allowed to dry after which they were incubated at 30° C. for 2 to 3 days and images were taken.

### 1.3.4.2 Growth Curves

Growth curves were measured of both yeast and human cell lines to assess the effect of Warbicins on general growth of the respective cell line.

(i) S. cerevisiae

Growth curves were made by measuring OD₅₉₅ in a 96-well plate using the Multiskan™ FC Microplate Photometer (Thermo Fisher Scientific). Typically, for tps1Δ rescue experiments (FIG. 2b-e; FIG. 4a), cells were (pre)grown in Complete Synthetic medium containing 2% galactose.

Compounds were always added 10 min prior to glucose addition to ensure maximal interaction between the cells and compounds. Cells originating from the preculture were harvested, washed in medium without sugar, after which they were resuspended to an OD of 0.1 in 200 μL of the appropriate medium. At least three technical replicates were included per condition. OD₅₉₅ was measured at 30° C. every 30 min with a 10-min shaking interval. For all growth curve experiments using S. cerevisiae, DMSO had a final concentration of 1%.

### (ii) Human Cell Lines

For both adherent A549 and MCF10Δ H-RAS^{V12} cell lines, growth curves were established using the IncuCyte® ZOOM technology. By collecting phase contrast images, the IncuCyte® software provides a calculated confluency percentage from which growth curves can be deducted.

Cells were seeded at a density of 1000-1500 cells/well in a Nunc-Edge™ 96-well plate (Thermo Fisher Scientific) and growth was typically measured over 3 to 5 days. For the measurement of apoptosis induction using the MCF10Δ H-RAS^{V12} cell line, the IncuCyte® Caspase-3/7 green dye was added to the medium in the recommended concentration at the beginning of the growth curve experiment. Apoptosis was detected by taking fluorescent images by excitation at 488 nm followed by IncuCyte® software analysis. Cell growth of the KMS-12-PE multiple myeloma cell line, which grows in suspension, was measured by manual cell counting. Cells were initially seeded at a density of 10⁴ cells/well and incubated for 4 days after which the cell number was counted. For all cell lines, the same medium used for pregrowth was also used for the growth curve experiment, albeit with different glucose concentrations depending on the experimental design. With compound administration, the final concentration of DMSO never exceeded 0.1%. Typically, at least four technical repeats were included per condition.

### 1.3.5 Metabolite Measurements

Metabolites were extracted and measured as described previously^{23,44}. General methods will be discussed in brief.

### (i) Sample Collection

For measuring metabolite levels, cells were typically grown in Complete Synthetic medium. To assess the effect of 10 mM glucose addition to tps1Δ and tps1Δ hxt6,7,4,2,5Δ cells (FIG. 1d-f), 3% glycerol was used as a carbon source. However, when comparing the effect of Warbicins on metabolite profiles of wild type and tps1Δ strains (FIG. 2f-k), cells were grown on 2% galactose. When grown to exponential phase, cells were harvested by centrifugation and washed twice with ice-cold 25 mM 2-(N-Morpholino)ethanesulfonic acid (MES) buffer, pH 6. Cells were suspended in Complete Synthetic medium without sugar at a concentration of 75 mg (wet weight)/mL followed by a temperature equilibration at 30° C. in a shaking water bath for 30 min. Depending on the experiment, a final concentration of 2.5 mM, 7.5 mM or 10 mM glucose was added to the cell suspension. Compounds were added 10 min prior to glucose addition. At distinct time points, 1.5 mL cell suspension was quenched in 60% methanol at −40° C.⁸⁹. Quenched samples were centrifuged at −10° C. followed by aspiration of the supernatant and resuspension of the cell pellet in 0.5 mL 1 M HCIO₄. After mechanical lysis of the cell suspensions by glass beads, an additional volume of 0.5 mL 1 M HCIO₄ was added after which the samples were stored at −20° C.

### (ii) Sample Processing

After centrifugation of the cell lysates at high speed, the supernatant fractions were collected. From here, lysate fractions were processed for neutralization. As such, to 250 μL cell lysate, 50 μL of 5 M K₂CO₃ was added, supplemented with 10 μL thymol blue (0.025%) to visually monitor the pH. After thorough mixing, samples were left to degas on ice for 15 min. Subsequently, samples were centrifuged at high speed from which 200 μL of the supernatant fraction was collected to which 100 μL 1 M HCl and 10 μL Tris-HCl (pH 7.5) was added. Samples were stored at −20° C.

(iii) Metabolite Measurement

Through endpoint measurement of the absorbance of NADH or NADPH at 340 nm, metabolite concentrations were calculated by applying Lambert's Law. Different metabolites were measured through the use of coupled enzymatic reactions. In general, 50 μL of sample was incubated with 150 μL assay buffer (100 mM Tris-HCl, pH 7.5). Depending on the measured metabolite, different co-factors and auxiliary enzymes were added. For measuring glucose-6-phosphate, 0.8 mg/ml NADP⁺ was added to the assay buffer after which the baseline absorbance was measured. The addition of 50 μg/mL Glu6P dehydrogenase oxidizes Glu6P while producing an equal amount of NADPH. After stabilization of the OD₃₄₀ spectra, ATP concentrations were measured by additionally adding 10 mM MgCl₂ and 0.5 mM glucose to the assay buffer. To start the enzymatic consumption of ATP, 100 μg/mL hexokinase was added. Finally, for measuring fructose-1,6-bisphosphate levels, 50 μL of sample was incubated with 150 μL assay buffer, supplemented with 8 μg/mL NADH, 25 μg/mL triosephosphate isomerase and 25 μg/mL glycerol-3-phosphate dehydrogenase. When NADH absorption was stable, 200 μg/mL aldolase was added to start the reaction. For all measured metabolites, after stabilization of the OD₃₄₀ spectra, the difference between the initial baseline value and the final absorbance after enzymatic conversion of the measured metabolite was used to determine its concentration. To express metabolite levels in terms of cytosolic concentration, an intracellular volume of 12 μL/mg protein was assumed.

### 1.3.6 Determination of Glucose Uptake Activity

### 1.3.6.1 Zero-Trans Uptake

(i) S. cerevisiae

Zero-trans uptake of radioactively labeled [U-¹⁴C]glucose, [U-¹⁴C]fructose or [U-¹⁴C]galactose by S. cerevisiae cells was determined in accordance with previous studies^{44,66}. Cells were grown on different carbon sources depending on the experiment. For measuring glucose uptake activity in the tps1Δ and tps1Δ hxtA strains (FIG. 1b,c), cells were grown on YP medium containing 3% glycerol (measurement of 10s). Typically, for compound characterization and kinetic studies in wild type and tps1Δ strains, cells were (pre)grown on Complete Synthetic medium supplemented with 2% galactose (measurement of 10s). In the experiments on the influence of hexokinase activity on the sugar uptake rate, the measurement duration was shortened to 5s and cells were always grown on YP medium containing 3% glycerol and 2% ethanol. After harvesting, cells were washed twice with ice-cold 25 mM MES-buffer pH 6, and resuspended in their respective medium without sugar to a final concentration of 45 mg (wet weight)/mL. The amount of added tracer was estimated to give a response close to at least 1000 counts per min in order to adequately counter background noise. Cells were first preincubated for 10 min at 30° C. with the compound to acclimate the cells to the temperature and to allow adequate interaction with the compound prior to uptake. Next, hexose sugar was mixed with the cell suspension, which was then incubated for 5 or 10 s, depending on the experiment, after which the cells were rapidly filtered over a glass microfiber filter (Whatman GF/C) and washed three times with ice-cold dH₂O. The loaded filter was transferred to a scintillation vial containing 3 mL liquid scintillation cocktail (Ultima-Flo M, Perkin Elmer) and counted using the Hidex 300 SL. Three blank measurements per strain were typically included to account for background signal, for which the cells were first quenched before adding the radioactive label.

### (ii) A549 Adenocarcinoma

Zero-trans glucose uptake in A549 cells was approximated by measuring the uptake of [1,2-³H]2-deoxyglucose. For this purpose, A549 cells were pregrown in RPMI medium containing 10 mM glucose in a 24-well plate to a cell density of around 100,000 cells/well. Prior to adding radioactive label, cells were gently washed twice in Krebs-Ringer-HEPES buffer (50 mM HEPES pH 7.4, 137 mM NaCl, 4.7 mM KCl, 1.85 CaCl₂, 1.3 mM MgSO₄ and 0.1% w/v BSA) at 30° C. to remove any residual sugar. RPMI medium without sugar containing the compound intended for treatment, was added to the cells for 15 min at 37° C. to allow adequate interaction of the cells with the compound.

The uptake measurement was initiated by adding an equal volume of medium containing radiolabeled 2DG. After 3-4 min, medium was aspirated and cells were gently washed three times with ice-cold Krebs-Ringer-HEPES buffer. Cells were lysed by adding 200 μL of ice-cold 0.1 M NaOH solution and incubating the plate for 10 min at 37° C. Cell lysates were transferred to scintillation vials for subsequent scintillation counting. For blank measurements, cells were incubated with 50 μM Cytochalasin B prior to the uptake measurement.

### 1.3.6.2 Steady-State Glucose Consumption Rate

### A549 Adenocarcinoma and KMS-12-PE Multiple Myeloma

The influence of WBC compounds on the steady-state glucose consumption rate was studied in the A549 adenocarcinoma and KMS-12-PE multiple myeloma cell lines. As such, different parameters needed to be optimized depending on whether cells were adherent or in suspension. As such, A549 cells were incubated at 125,000 cells/well in a 24-well plate in 300 μL RPMI medium for 8 h. For the KMS-12-PE cell line, 100,000 cells/well were incubated in 100 μL RPMI medium in a 96-well plate for 8 h. Medium was collected, spun down and HPLC-analyzed for measuring glucose and lactate levels. Metabolite levels were corrected for cell number, which always varied little over the span of 8 h. For every condition, at least 4 technical repeats were included.

### 1.3.7 Fluorescence Microscopy

Using fluorescence microscopy, both the localization of individual proteins as well as BiFC interactions were studied by genomic C-terminal tagging of proteins of interest with full-length or split Citrine halves (NCitr: Citrine^{AA1-154} CCitr: Citrine^{AA155-236}) or by plasmid-based expression of fluorescent fusion proteins. The mCitrine fluorophore has an excitation and emission maximum at 516 and 529 nm, respectively. Using the Olympus FluoView™ 1000 confocal laser microscope, cells were excited by the 515 nm laser line with DM458/515 and emission was monitored using the band pass BA535-565 filter set. Images were scanned at 8.0 ρs/pixel combined with a 60× oil objective lens (Olympus UPIanSAPO, N.A. 1.35) together with a digital zoom of 5×. For fluorescence microscopy assays, cells were typically (pre)grown on YP containing 3% glycerol and 2% ethanol. A small sample was taken from the mother culture, spun down at 2000 rpm and resuspended in a smaller volume to concentrate the cells. Next, 5 μL of cell suspension was applied to a glass slide and sealed by a coverslip after which the slide was allowed to settle for at least 5-10 min prior to visualization.

### 1.3.8 Pulldown Followed by Western Blot Analysis

### (i) Purification of GST-Hxk2

For expression of GST and GST-Hxk2 in E. coli BL21, cells were grown in LB to exponential phase at 37° C. and subsequently induced by 0.3 mM IPTG at room temperature overnight. Cell pellets were harvested and washed in ice-cold 25 mM MES-buffer pH 6. Next, cells were resuspended in lysis buffer (50 mM Tris-HCl pH 7.5, 150 mM NaCl, 1 mM EDTA, 2.5 mM MgCl₂) for 30 min on ice containing 5 mg/mL lysozyme for cell wall digestion. After incubation, three additional volumes of lysis buffer were added containing 1% Triton X-100 and protease inhibitor cocktail (Roche) followed by three cycles of sonication with intermediate pauses on ice. Cell lysates were clarified by centrifugation at 10,000 rcf and incubated with Glutathione Sepharose™ 4B resin (GE Healthcare). As such, beads were incubated with cell lysate for 1 to 2 h on a roller drum at 4° C. followed by three wash steps with lysis buffer containing 1% Triton X-100.

### (ii) Pulldown of Hxt7-HA

Wild type cells transformed with pHX77-HA were grown on uracil-deficient medium containing 2% galactose until exponential phase. Cells were harvested, washed with 25 mM MES pH 6, and resuspended in lysis buffer (50 mM Tris-HCl pH 7.5, 150 mM NaCl, 5% glycerol, 1 mM EDTA, 2.5 mM MgCl₂, 1% Triton X-100) supplemented with protease inhibitor cocktail. Crude extracts were obtained by mechanically lysing cells by fast-prepping 3 times for 20 s (6 m/s) with intermediate pauses on ice. Cells were centrifuged at 10,000×rcf and the supernatant was collected of which the protein concentration was determined. On average, 1 mg protein extract was incubated with 25 μL GST(-Hxk2)-coated beads overnight on a roller drum at 4° C. Finally, beads were washed three times with lysis buffer and the final bead pellet was resuspended in 100 μL 2× loading dye (4% SDS, 100 mM Tris-HCl pH 6.8, 10% glycerol, 0.02% bromophenol blue) and heated at 45° C. for 10 min after which the samples were stored at −20° C. For the Hxt7-HA input sample, a fraction of protein extract was immediately added to 2× loading dye, heated at 45° C. and stored at −20° C.

(iii) Western Blot Analysis

For western blot analysis, 10 μg of input and 10 μL of pulldown samples were loaded on a 4-12% Bis-Tris NuPage™ gel, in duplo. Gels were run at a constant voltage of 120-150 V in NuPage™ MOPS SDS running buffer. To verify the presence of GST and GST-Hxk2 in the pulldown samples, the first gel was incubated in 60 mg/mL Coomassie Blue Brilliant solution in 10% acetate for 30 min and washed several times with TBS-T buffer (25 mM Tris-HCl pH 8, 150 mM NaCl, 0.05% v/v Tween-20) until gels became clear. The second gel was blotted in NuPage™ MOPS SDS blotting buffer containing 20% (v/v) methanol at a constant 300 mA for 1.5 h. After blotting, the nitrocellulose membrane (Hybond-C extra, GE healthcare) was blocked in 5% (w/v) skimmed milk powder dissolved in TBS-T buffer overnight at 4° C. Next, the membrane was immune-labeled with 1:1000 anti-HA (Roche) and washed three times with TBS-T to prepare the membrane for chemiluminescence detection. For this, SuperSignal West Pico PLUS Chemiluminescent substrate (Pierce®) was added onto the nitrocellulose membrane and incubated for 2 min in the dark prior to visualization. Chemiluminescence detection was performed using the ImageQuant™ LAS4000 mini digital system.

### 1.3.9 Determination of Hexokinase Activity

In vitro hexokinase activity was determined as described previously 43, s. As such, cells were first grown to exponential phase on complete Synthetic medium supplemented with 2% galactose. The cells were harvested and washed with ice-cold 25 mM MES buffer pH 6. Subsequently, cells were resuspended in lysis buffer (50 mM HEPES pH 7, 150 mM NaCl, 2.5 mM MgCl₂, 5% glycerol and 1% Triton X-100) containing protease inhibitor after which the cells were mechanically lysed. After protein level determination, total protein concentration was diluted to 0.1 mg/mL in reaction buffer (50 mM HEPES pH 7, 150 mM NaCl, 2.5 mM MgCl₂, 5% glycerol). For measuring activity, reaction buffer was supplemented with 0.8 mg/mL NADP⁺ and 50 μg/mL Glu6P dehydrogenase. After temperature equilibration of the plate at 30° C., the reaction was started by adding both ATP and glucose to the reaction buffer. OD₃₄₀ was measured using the Synergy H1 Hybrid reader and hexokinase activity was determined based on the linear increase of absorbance in the first 10-20s.

### 1.3.10 Evaluation of Warbicin Toxicity In Vivo

Warbicin in vivo toxicity and tolerability was examined by subjecting NMRI-nu mice to daily intraperitoneal injection of either WBC-A, WBC-15C, WBC-4C or WBC-11C. Compounds were dosed at either 5 mg/kg, 10 mg/kg or 20 mg/kg over a period of 20 days. Three mice were used per condition. To evaluate toxicity, change in body weight was registered daily. As a humane endpoint, mice that lost more than 20% of their original body weight were prematurely sacrificed. An optimal dissolve strategy was developed to administer WBC compounds by intraperitoneal injection. Due to their considerable hydrophobicity, compounds were dissolved in a sterilized 1×PBS, 5% DMSO and 5% Tween-80 solution. For WBC-A, -15C and -4C, this gives an initial clear solution that gradually changes over time into a homogenous suspension which can still be administered in a reproducible way. For WBC-11C it immediately results in a homogenous suspension.

### Example 2

Evaluation of the Inhibitory Effect of the Warbicin® Compounds on Tumor Growth in Xenograft Experiments with Mice.

Female NMRI nude mice of 8 weeks old were inoculated with the A549 cancer cell line and tumors allowed to grow for 35 days to an average volume of 100 mm³.

Compounds were administered by intraperitoneal injection of different concentrations of Warbicin® compounds. The injection volume was 200 μL. The final compound concentration was: 3 mg/mL for 20 mg/kg body weight; 1.5 mg/mL for 10 mg/kg body weight; 0.375 mg/mL for 2.5 mg/kg body weight.

The Warbicin® compounds were aliquoted in 75 μL of 5% DMSO in 2 mL Eppendorf tubes and stored at −20 0C. For administration, 75 μL of 5% Tween 80 and 1350 μL of 90% PBS were added to make a final volume of 1.5 mL of which 200 μL was used for intraperitoneal injection.

The average mouse weight was 30 g. If the mouse weight differed from the average, the injection volume was adjusted accordingly to maintain the correct dosage (mg compound/kg mouse).

In each group 5 mice were used:
Group 1: 5 mice vehicle (DMSO)
Group 2: 5 mice treated with compound A (20 mg/kg/daily)
Group 3: 5 mice treated with compound A (10 mg/kg/daily)
Group 4: 5 mice treated with compound A (2.5 mg/kg/daily)
Group 5: 5 mice treated with compound 4C (20 mg/kg/daily)
Group 6: 5 mice treated with compound 4C (10 mg/kg/daily)
Group 7: 5 mice treated with compound 4C (2.5 mg/kg/daily)
Group 8: 5 mice treated with compound 11C (2.5 mg/kg/daily)

The Warbicin® compounds caused a significant retardation of the tumor growth (FIG. 24).

Warbicin A had a clear dose-dependent inhibitory effect for the three concentrations used. Warbicin 4 caused inhibition for all concentrations and Warbicin 11C inhibited at 2.5 mg/kg.

No significant adverse effect on body weight was observed for none of the conditions during the treatment period with the Warbicin® compounds (FIG. 25).

TABLE 5
Molecular structure of the 21 analogs of WBC-A that rescue to different extent growth on
glucose of tps1Δ cells.

| Name | Structure | | |
|---|---|---|---|
| Conserved backbone | 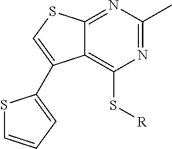 | | |
| | | | Minimal rescue |
| | | IC₅₀ values for | concentration for |
| | | 2.5 mM glucose | growth of tps1Δ |
| | | uptake in tps1Δ | cells on 2.5 mM |
| R-group | Structure | cells (μM) | glucose (μM) |
| WBC-2C | 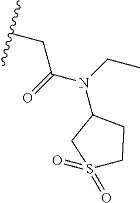 [SMILES: *CC(=O)N(CC)C1CCS(=O)(=O)C1] | 2.0 | 1.562 |
| WBC-26A | 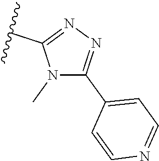 [SMILES: *c1nnc(-c2ccncc2)n1C] | 5.35 | 25 |
| WBC-7B | 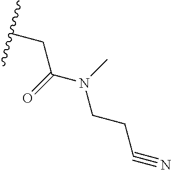 [SMILES: *CC(=O)N(C)CCC#N] | 5.41 | 6.25 |
| WBC-23A | 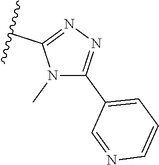 [SMILES: *c1nnc(-c2cccnc2)n1C] | 8.60 | 25 |
| WBC-43C | 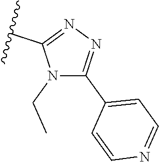 [SMILES: *c1nnc(-c2ccncc2)n1CC] | 8.93 | 12.5 |
| WBC-29B | 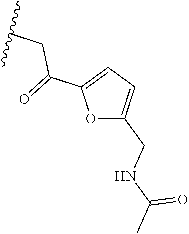 [SMILES: *CC(=O)c1ccc(CNC(C)=O)o1] | 9.23 | 6.25 |
| WBC-6B | 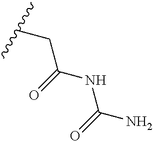 [SMILES: *CC(=O)NC(N)=O] | 10.17 | 12.5 |
| WBC-87C | 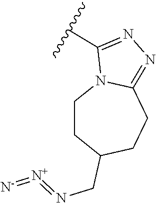 [SMILES: *c1nnc2n1CCC(CN=[N+]=[N-])CC2] | 11.08 | 12.5 |
| WBC-47A | 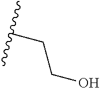 [SMILES: *CCO] | 15.59 | 6.25 |
| WBC-A | 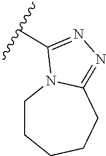 [SMILES: *c1nnc2n1CCCCC2] | 20.75 | 25 |
| WBC-5B | 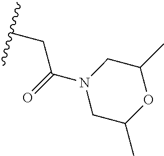 [SMILES: *CC(=O)N1CC(C)OC(C)C1] | 22.51 | 12.5 |
| WBC-26C | 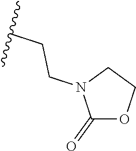 [SMILES: *CCN1CCOC1=O] | 23.82 | 25 |
| WBC-10C | 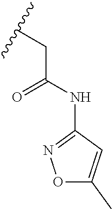 [SMILES: *CC(=O)Nc1cc(C)on1] | 24.81 | 6.25 |
| WBC-53A | 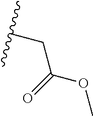 [SMILES: *CC(=O)OC] | 26.75 | 12.5 |
| WBC-14A | 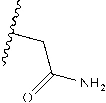 [SMILES: *CC(N)=O] | 27.94 | 25 |
| WBC-32B | 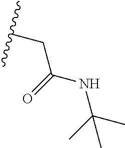 [SMILES: *CC(=O)NC(C)(C)C] | 30.29 | 12.5 |
| WBC-15B | 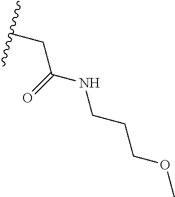 [SMILES: *CC(=O)NCCCOC] | 32.72 | 25 |
| WBC-11C | 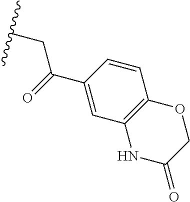 [SMILES: *CC(=O)c1ccc2c(c1)NC(=O)CO2] | 44.01 | 50 |
| WBC-16C | 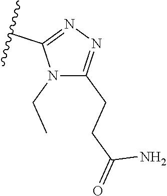 [SMILES: *c1nnc(CCC(N)=O)n1CC] | 49.62 | 50 |
| WBC-44A | 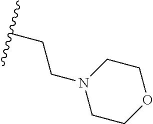 [SMILES: *CCN1CCOCC1] | 61.75 | 50 |
| WBC-54C | 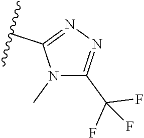 [SMILES: *c1nnc(C(F)(F)F)n1C] | 64.66 | 50 |
| WBC-50C | 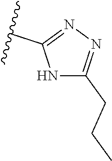 [SMILES: *c1nnc(CCC)[nH]1] | 67.77 | 50 |

For every compound, both the IC50 value for 2.5 mM glucose transport inhibition and the minimal rescue concentration for growth of the tps1Δ strain on 2.5 mM glucose are shown. The common backbone structure is illustrated of which the R-group denotes the compound-specific side-chain.

TABLE 6
Overview of the molecular structure of WBC-A analogs and their bioactivity with respect to growth rescue of the
tps1Δ strain and growth inhibition of the A549 cell line.

| | | | Rescue | | Selected for |
|---|---|---|---|---|---|
| | | | of the | Selected from | having an |
| | | | tps1Δ | primary | IC₅₀ ratio |
| | | | strain on | screen | (10 mM |
| | | | 2.5 mM | for growth | glucose: 1 mM |
| | Compound | | glucose | inhibition of | glucose) of A549 |
| Name: | vendor | | at 100 | A549 cells at | growth higher |
| WBC- | and code | Structure | μM | 50 μM | than WBC-A |
| A | Enamine, Z85926102 | 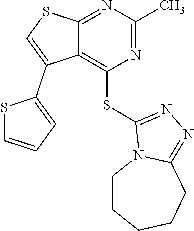 [SMILES: Cc1nc(Sc2nnc3n2CCCCC3)c2c(-c3cccs3)csc2n1] | Yes | Yes | Used as cutoff threshold |
| 1A | Enamine, Z57986816 | 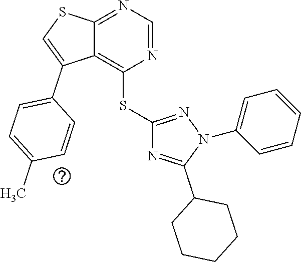 [SMILES: Cc1ccc(-c2csc3ncnc(Sc4nc(C5CCCCC5)n(-c5ccccc5)n4)c23)cc1] | No | No | Not tested |
| 2A | Enamine, Z85925971 | 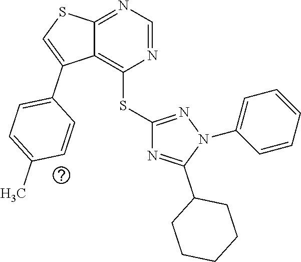 [SMILES: Cc1ccc(-c2csc3ncnc(Sc4nc(C5CCCCC5)n(-c5ccccc5)n4)c23)cc1] | No | No | Not tested |
| 3A | Enamine, Z85934181 | 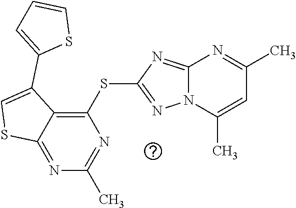 [SMILES: Cc1cc(C)n2nc(Sc3nc(C)nc4scc(-c5cccs5)c34)nc2n1] | No | No | Not tested |
| 4A | Enamine, Z2239062993 | 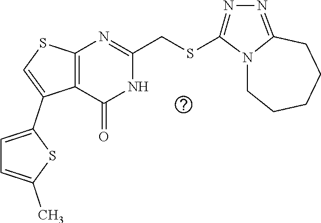 [SMILES: Cc1ccc(-c2csc3nc(CSc4nnc5n4CCCCC5)[nH]c(=O)c23)s1] | No | No | Not tested |
| 5A | Enamine, Z31109634 | 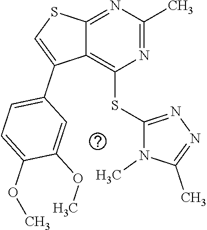 [SMILES: COc1ccc(-c2csc3nc(C)nc(Sc4nnc(C)n4C)c23)cc1OC] | No | No | Not tested |
| 6A | Enamine, Z21579098 | 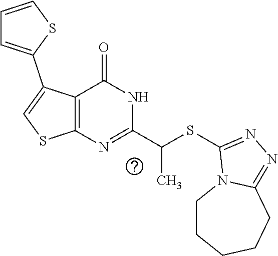 [SMILES: CC(Sc1nnc2n1CCCCC2)c1nc2scc(-c3cccs3)c2c(=O)[nH]1] | No | No | Not tested |
| 7A | Enamine, Z212114188 | 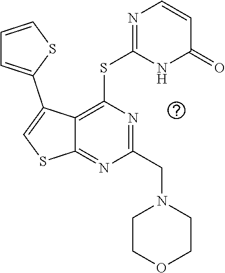 [SMILES: O=c1ccnc(Sc2nc(CN3CCOCC3)nc3scc(-c4cccs4)c23)[nH]1] | No | No | Not tested |
| 8A | Enamine, Z198754772 | 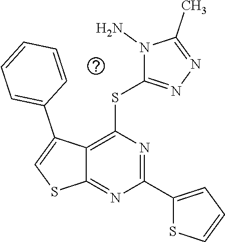 [SMILES: Cc1nnc(Sc2nc(-c3cccs3)nc3scc(-c4ccccc4)c23)n1N] | No | No | Not tested |
| 9A | Enamine, Z2239070437 | 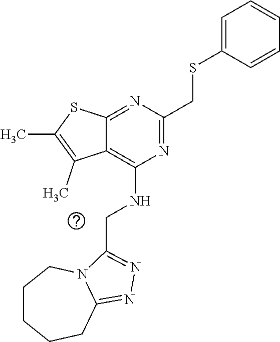 [SMILES: Cc1sc2nc(CSc3ccccc3)nc(NCc3nnc4n3CCCCC4)c2c1C] | No | No | Not tested |
| 10A | Enamine, Z234870892 | 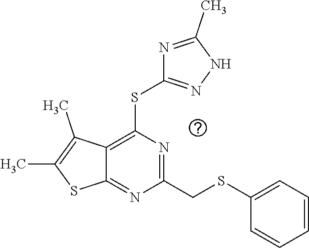 [SMILES: Cc1nc(Sc2nc(CSc3ccccc3)nc3sc(C)c(C)c23)n[nH]1] | No | No | Not tested |
| 11A | Enamine, Z193493822 | 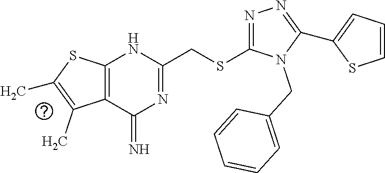 [SMILES: Cc1sc2[nH]c(CSc3nnc(-c4cccs4)n3Cc3ccccc3)nc(=N)c2c1C] | No | No | Not tested |
| 12A | Enamine, Z199929046 | 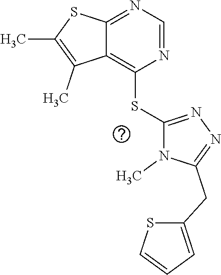 [SMILES: Cc1sc2ncnc(Sc3nnc(Cc4cccs4)n3C)c2c1C] | No | No | Not tested |
| 13A | Enamine, Z2239072210 | 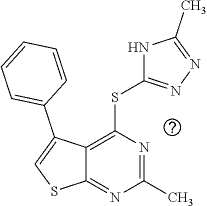 [SMILES: Cc1nc(Sc2nnc(C)[nH]2)c2c(-c3ccccc3)csc2n1] | No | No | Not tested |
| 14A | Enamine, Z16637978 | 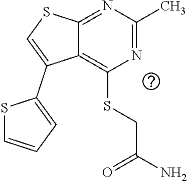 [SMILES: Cc1nc(SCC(N)=O)c2c(-c3cccs3)csc2n1] | Yes | No | Not tested |
| 15A | Enamine, Z16638026 | 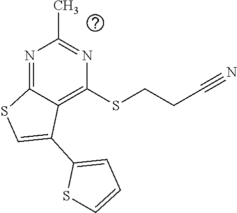 [SMILES: Cc1nc(SCCC#N)c2c(-c3cccs3)csc2n1] | No | No | Not tested |
| 16A | Enamine, Z31109644 | 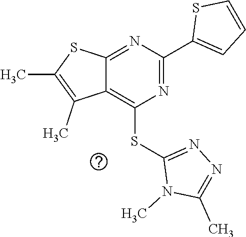 [SMILES: Cc1sc2nc(-c3cccs3)nc(Sc3nnc(C)n3C)c2c1C] | No | No | Not tested |
| 17A | Enamine, Z275103620 | 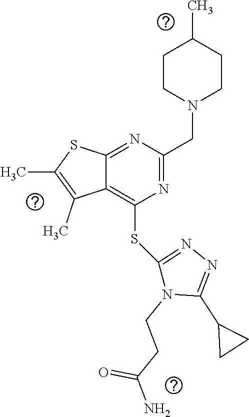 [SMILES: Cc1sc2nc(CN3CCC(C)CC3)nc(Sc3nnc(C4CC4)n3CCC(N)=O)c2c1C] | No | Yes | Lower IC₅₀ ratio |
| 18A | Enamine, Z16637998 | 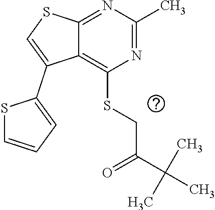 [SMILES: Cc1nc(SCC(=O)C(C)(C)C)c2c(-c3cccs3)csc2n1] | No | Yes | Lower IC₅₀ ratio |
| 19A | Enamine, Z31109564 | 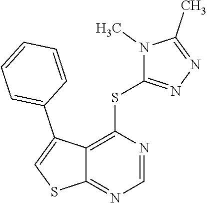 [SMILES: Cc1nnc(Sc2ncnc3scc(-c4ccccc4)c23)n1C] | No | No | Not tested |
| 20A | Enamine, Z199817504 | 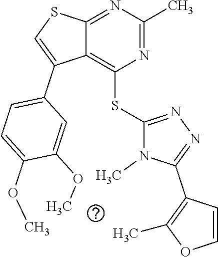 [SMILES: COc1ccc(-c2csc3nc(C)nc(Sc4nnc(-c5ccoc5C)n4C)c23)cc1OC] | No | Yes | Lower IC₅₀ ratio |
| 21A | Enamine, Z200072422 | 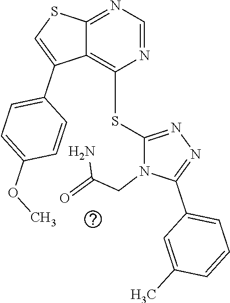 [SMILES: COc1ccc(-c2csc3ncnc(Sc4nnc(-c5cccc(C)c5)n4CC(N)=O)c23)cc1] | No | Yes | Lower IC₅₀ ratio |
| 22A | Enamine, Z220377504 | 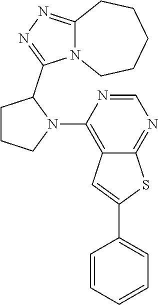 [SMILES: c1ccc(-c2cc3c(N4CCCC4c4nnc5n4CCCCC5)ncnc3s2)cc1] | No | No | Not tested |
| 23A | Enamine, Z199805674 | 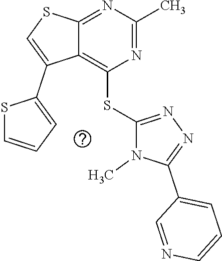 [SMILES: Cc1nc(Sc2nnc(-c3cccnc3)n2C)c2c(-c3cccs3)csc2n1] | Yes | Yes | Lower IC₅₀ ratio |
| 24A | Enamine, Z198006244 | 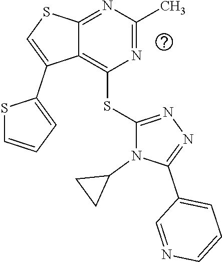 [SMILES: Cc1nc(Sc2nnc(-c3cccnc3)n2C2CC2)c2c(-c3cccs3)csc2n1] | No | Yes | Lower IC₅₀ ratio |
| 25A | Enamine, Z200045206 | 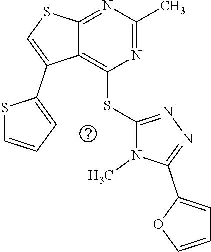 [SMILES: Cc1nc(Sc2nnc(-c3ccco3)n2C)c2c(-c3cccs3)csc2n1] | No | No | Not tested |
| 26A | Enamine, Z199723150 | 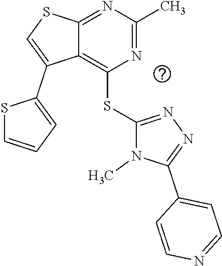 [SMILES: Cc1nc(Sc2nnc(-c3ccncc3)n2C)c2c(-c3cccs3)csc2n1] | Yes | Yes | Lower IC₅₀ ratio |
| 27A | Enamine, Z200026606 | 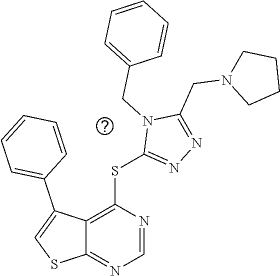 [SMILES: c1ccc(Cn2c(CN3CCCC3)nnc2Sc2ncnc3scc(-c4ccccc4)c23)cc1] | No | Yes | Lower IC₅₀ ratio |
| 28A | Enamine, Z198001832 | 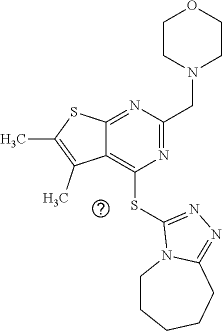 [SMILES: Cc1sc2nc(CN3CCOCC3)nc(Sc3nnc4n3CCCCC4)c2c1C] | No | No | Not tested |
| 29A | Enamine, Z64619643 | 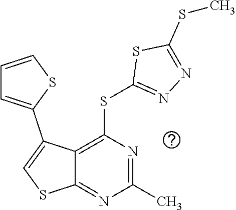 [SMILES: CSc1nnc(Sc2nc(C)nc3scc(-c4cccs4)c23)s1] | No | No | Not tested |
| 30A | Enamine, Z31083015 | 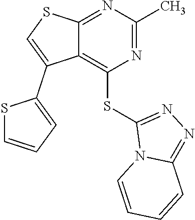 [SMILES: Cc1nc(Sc2nnc3ccccn23)c2c(-c3cccs3)csc2n1] | No | No | Not tested |
| 31A | Enamine, Z198001782 | 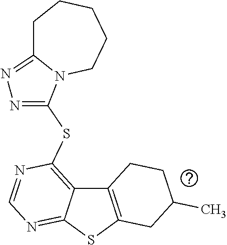 [SMILES: CC1CCc2c(sc3ncnc(Sc4nnc5n4CCCCC5)c23)C1] | No | No | Not tested |
| 32A | Enamine, Z200173686 | 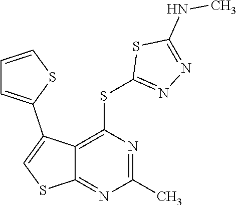 [SMILES: CNc1nnc(Sc2nc(C)nc3scc(-c4cccs4)c23)s1] | No | No | Not tested |
| 33A | Enamine, Z645064812 | 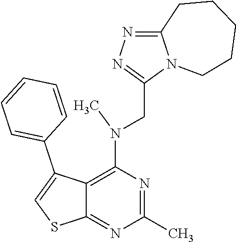 [SMILES: Cc1nc(N(C)Cc2nnc3n2CCCCC3)c2c(-c3ccccc3)csc2n1] | No | No | Not tested |
| 34A | Enamine, Z645064508 | 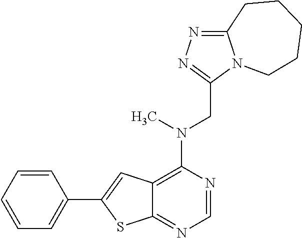 [SMILES: CN(Cc1nnc2n1CCCCC2)c1ncnc2sc(-c3ccccc3)cc12] | No | No | Not tested |
| 35A | Enamine, Z193725386 | 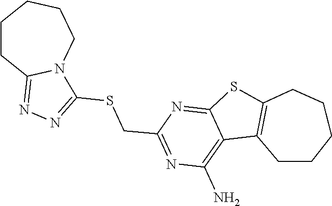 [SMILES: Nc1nc(CSc2nnc3n2CCCCC3)nc2sc3c(c12)CCCCC3] | No | Yes | Lower IC₅₀ ratio |
| 36A | Enamine, Z645064922 | 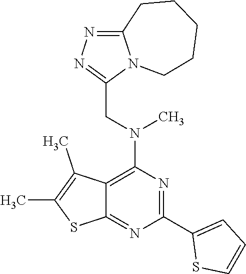 [SMILES: Cc1sc2nc(-c3cccs3)nc(N(C)Cc3nnc4n3CCCCC4)c2c1C] | No | No | Not tested |
| 37A | Enamine, Z1334451862 | 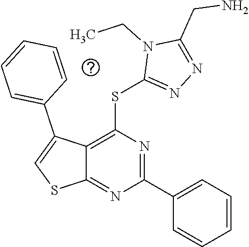 [SMILES: CCn1c(CN)nnc1Sc1nc(-c2ccccc2)nc2scc(-c3ccccc3)c12] | No | No | Not tested |
| 38A | Enamine, Z361946650 | 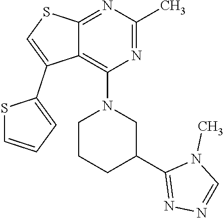 [SMILES: Cc1nc(N2CCCC(c3nncn3C)C2)c2c(-c3cccs3)csc2n1] | No | No | Not tested |
| 39A | Enamine, Z31243547 | 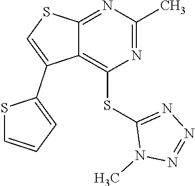 [SMILES: Cc1nc(Sc2nnnn2C)c2c(-c3cccs3)csc2n1] | No | Yes | Lower IC₅₀ ratio |
| 40A | Enamine, Z1383881908 | 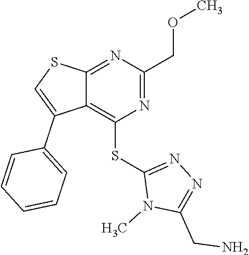 [SMILES: COCc1nc(Sc2nnc(CN)n2C)c2c(-c3ccccc3)csc2n1] | No | No | Not tested |
| 41A | Enamine, Z1526936211 | 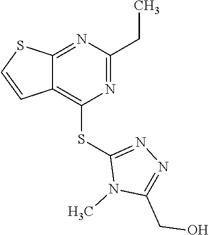 [SMILES: CCc1nc(Sc2nnc(CO)n2C)c2ccsc2n1] | No | Yes | Lower IC₅₀ ratio |
| 42A | Enamine, Z31241654 | 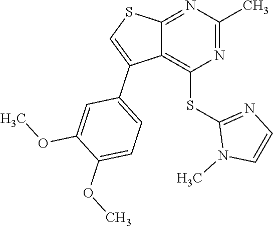 [SMILES: COc1ccc(-c2csc3nc(C)nc(Sc4nccn4C)c23)cc1OC] | No | Yes | Lower IC₅₀ ratio |
| 43A | Enamine, Z31243444 | 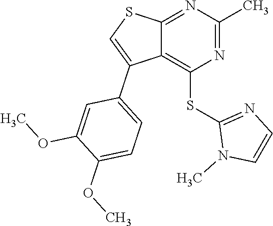 [SMILES: COc1ccc(-c2csc3nc(C)nc(Sc4nccn4C)c23)cc1OC] | No | Yes | Lower IC₅₀ ratio |
| 44A | Enamine, Z16637947 | 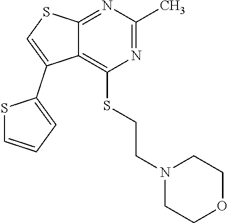 [SMILES: Cc1nc(SCCN2CCOCC2)c2c(-c3cccs3)csc2n1] | Yes | Yes | Lower IC₅₀ ratio |
| 45A | Enamine, Z16637970 | 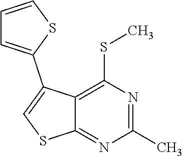 [SMILES: CSc1nc(C)nc2scc(-c3cccs3)c12] | No | No | Not tested |
| 46A | Enamine, Z31082970 | 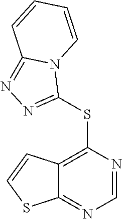 [SMILES: c1ccn2c(Sc3ncnc4sccc34)nnc2c1] | No | No | Not tested |
| 47A | Enamine, Z31206481 | 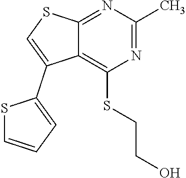 [SMILES: Cc1nc(SCCO)c2c(-c3cccs3)csc2n1] | Yes | Yes | Lower IC₅₀ ratio |
| 48A | Enamine, Z31243469 | 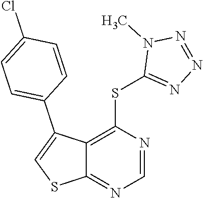 [SMILES: Cn1nnnc1Sc1ncnc2scc(-c3ccc(Cl)cc3)c12] | No | Yes | Lower IC₅₀ ratio |
| 49A | Enamine, Z31082948 | 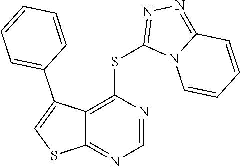 [SMILES: c1ccc(-c2csc3ncnc(Sc4nnc5ccccn45)c23)cc1] | No | Yes | Lower IC₅₀ ratio |
| 50A | Enamine, Z57990683 | 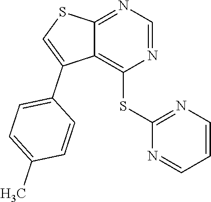 [SMILES: Cc1ccc(-c2csc3ncnc(Sc4ncccn4)c23)cc1] | No | No | Not tested |
| 51A | Enamine, Z2239055447 | 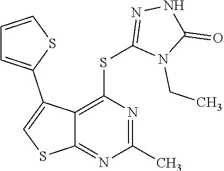 [SMILES: CCn1c(Sc2nc(C)nc3scc(-c4cccs4)c23)n[nH]c1=O] | No | Yes | Lower IC₅₀ ratio |
| 52A | Enamine, Z85926207 | 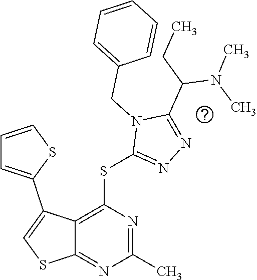 [SMILES: CCC(c1nnc(Sc2nc(C)nc3scc(-c4cccs4)c23)n1Cc1ccccc1)N(C)C] | No | Yes | Lower IC₅₀ ratio |
| 53A | Enamine, Z16637984 | 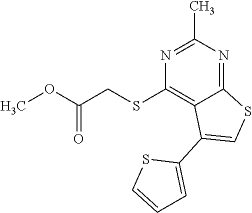 [SMILES: COC(=O)CSc1nc(C)nc2scc(-c3cccs3)c12] | Yes | No | Not tested |
| 54A | Enamine, Z31243549 | 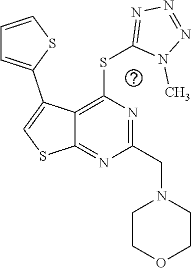 [SMILES: Cn1nnnc1Sc1nc(CN2CCOCC2)nc2scc(-c3cccs3)c12] | No | Yes | Lower IC₅₀ ratio |
| 55A | Enamine, Z94734908 | 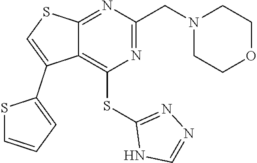 [SMILES: c1csc(-c2csc3nc(CN4CCOCC4)nc(Sc4nnc[nH]4)c23)c1] | Yes | No | Not tested |
| 56A | Enamine, Z128124392 | 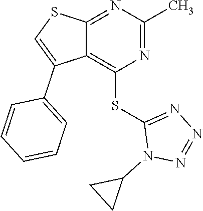 [SMILES: Cc1nc(Sc2nnnn2C2CC2)c2c(-c3ccccc3)csc2n1] | No | Yes | Lower IC₅₀ ratio |
| 57A | Enamine, Z31243468 | 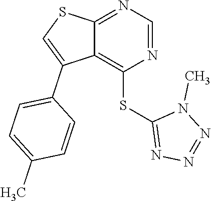 [SMILES: Cc1ccc(-c2csc3ncnc(Sc4nnnn4C)c23)cc1] | No | Yes | Lower IC₅₀ ratio |
| 58A | Enamine, Z31082949 | 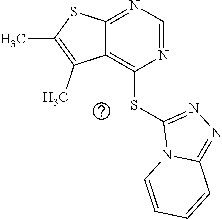 [SMILES: Cc1sc2ncnc(Sc3nnc4ccccn34)c2c1C] | No | No | Not tested |
| 59A | Enamine, Z56930170 | 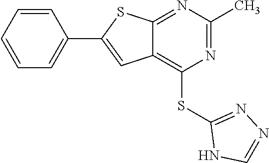 [SMILES: Cc1nc(Sc2nnc[nH]2)c2cc(-c3ccccc3)sc2n1] | No | No | Not tested |
| 60A | Enamine, Z31082966 | 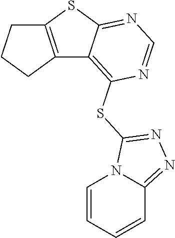 [SMILES: c1ccn2c(Sc3ncnc4sc5c(c34)CCC5)nnc2c1] | No | No | Not tested |
| 61A | Enamine, Z128193468 | 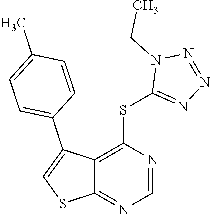 [SMILES: CCn1nnnc1Sc1ncnc2scc(-c3ccc(C)cc3)c12] | No | Yes | Lower IC₅₀ ratio |
| 62A | Enamine, Z56777524 | 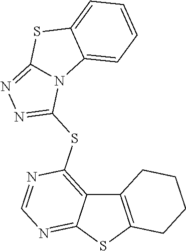 [SMILES: c1ccc2c(c1)sc1nnc(Sc3ncnc4sc5c(c34)CCCC5)n12] | No | No | Not tested |
| 63A | Enamine, Z198001598 | 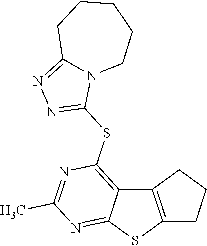 [SMILES: Cc1nc(Sc2nnc3n2CCCCC3)c2c3c(sc2n1)CCC3] | No | No | Not tested |
| 64A | Enamine, Z198001640 | 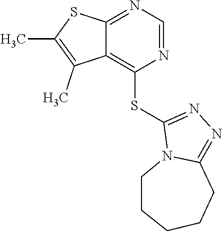 [SMILES: Cc1sc2ncnc(Sc3nnc4n3CCCCC4)c2c1C] | No | No | Not tested |
| 65A | Enamine, Z198001628 | 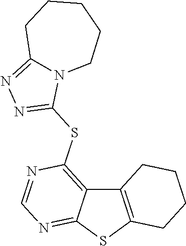 [SMILES: c1nc(Sc2nnc3n2CCCCC3)c2c3c(sc2n1)CCCC3] | No | No | Not tested |
| 66A | Enamine, Z275103616 | 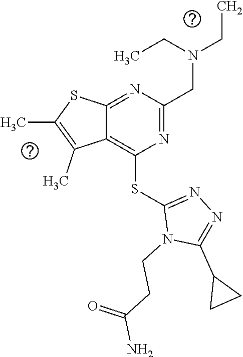 [SMILES: CCN(CC)Cc1nc(Sc2nnc(C3CC3)n2CCC(N)=O)c2c(C)c(C)sc2n1] | No | No | Not tested |
| 67A | Enamine, Z2239050295 | 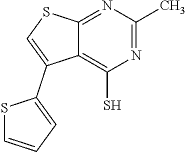 [SMILES: Cc1nc(S)c2c(-c3cccs3)csc2n1] | No | No | Not tested |
| 1B | Enamine, Z16638342 | 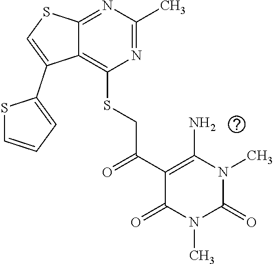 [SMILES: Cc1nc(SCC(=O)c2c(N)n(C)c(=O)n(C)c2=O)c2c(-c3cccs3)csc2n1] | No | No | Not tested |
| 2B | Enamine, Z26695654 | 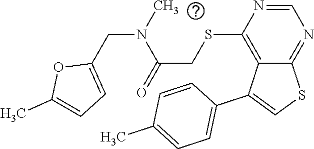 [SMILES: Cc1ccc(-c2csc3ncnc(SCC(=O)N(C)Cc4ccc(C)o4)c23)cc1] | No | Yes | Lower IC₅₀ ratio |
| 3B | Enamine, Z31139179 | 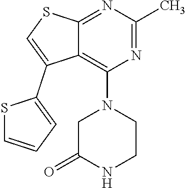 [SMILES: Cc1nc(N2CCNC(=O)C2)c2c(-c3cccs3)csc2n1] | No | No | Not tested |
| 4B | Enamine, Z103172970 | 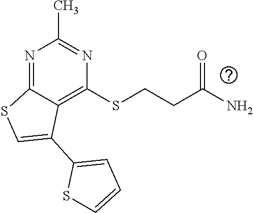 [SMILES: Cc1nc(SCCC(N)=O)c2c(-c3cccs3)csc2n1] | No | No | Not tested |
| 5B | Enamine, Z16638356 | 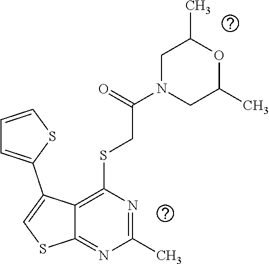 [SMILES: Cc1nc(SCC(=O)N2CC(C)OC(C)C2)c2c(-c3cccs3)csc2n1] | Yes | No | Not tested |
| 6B | Enamine, Z16637427 | 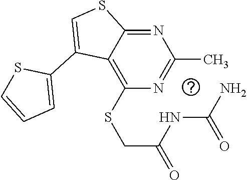 [SMILES: Cc1nc(SCC(=O)NC(N)=O)c2c(-c3cccs3)csc2n1] | Yes | Yes | Lower IC₅₀ ratio |
| 7B | Enamine, Z16637291 | 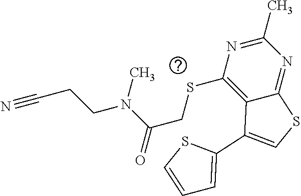 [SMILES: Cc1nc(SCC(=O)N(C)CCC#N)c2c(-c3cccs3)csc2n1] | Yes | No | Not tested |
| 8B | Enamine, Z24279401 | 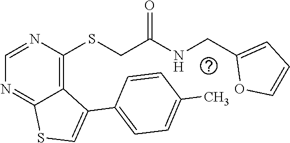 [SMILES: Cc1ccc(-c2csc3ncnc(SCC(=O)NCc4ccco4)c23)cc1] | No | Yes | Lower IC₅₀ ratio |
| 9B | Enamine, Z16637398 | 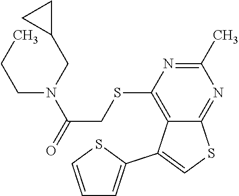 [SMILES: CCCN(CC1CC1)C(=O)CSc1nc(C)nc2scc(-c3cccs3)c12] | No | No | Not tested |
| 10B | Enamine, Z52577815 | 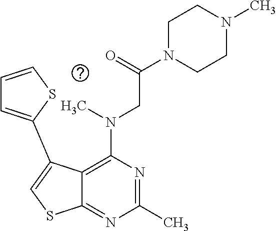 [SMILES: Cc1nc(N(C)CC(=O)N2CCN(C)CC2)c2c(-c3cccs3)csc2n1] | No | No | Not tested |
| 11B | Enamine, Z20246006 | 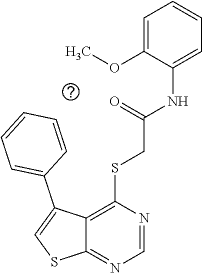 [SMILES: COc1ccccc1NC(=O)CSc1ncnc2scc(-c3ccccc3)c12] | No | No | Not tested |
| 12B | Enamine, Z56882642 | 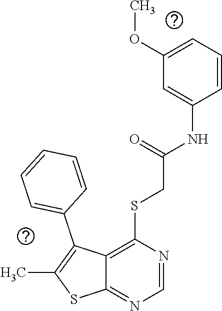 [SMILES: COc1cccc(NC(=O)CSc2ncnc3sc(C)c(-c4ccccc4)c23)c1] | No | Yes | Lower IC₅₀ ratio |
| 13B | Enamine, Z16637043 | 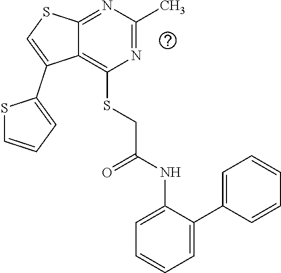 [SMILES: Cc1nc(SCC(=O)Nc2ccccc2-c2ccccc2)c2c(-c3cccs3)csc2n1] | No | Yes | Higher IC₅₀ ratio, but false-positive |
| 14B | Enamine, Z16637598 | 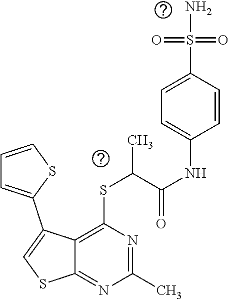 [SMILES: Cc1nc(SC(C)C(=O)Nc2ccc(S(N)(=O)=O)cc2)c2c(-c3cccs3)csc2n1] | No | Yes | Lower IC₅₀ ratio |
| 15B | Enamine, Z16637300 | 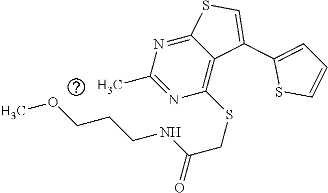 [SMILES: COCCCNC(=O)CSc1nc(C)nc2scc(-c3cccs3)c12] | Yes | No | Not tested |
| 16B | Enamine, Z2241109880 | 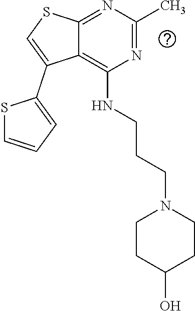 [SMILES: Cc1nc(NCCCN2CCC(O)CC2)c2c(-c3cccs3)csc2n1] | No | No | Not tested |
| 17B | Enamine, Z16637464 | 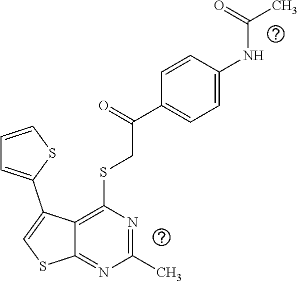 [SMILES: CC(=O)Nc1ccc(C(=O)CSc2nc(C)nc3scc(-c4cccs4)c23)cc1] | No | Yes | Higher IC₅₀ ratio, but false-positive |
| 18B | Enamine, Z16637803 | 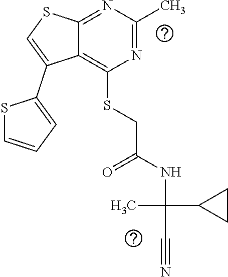 [SMILES: Cc1nc(SCC(=O)NC(C)(C#N)C2CC2)c2c(-c3cccs3)csc2n1] | No | Yes | Higher IC₅₀ ratio, but false-positive |
| 19B | Enamine, Z16638051 | 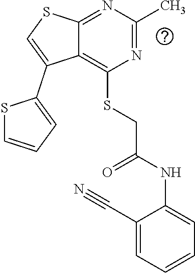 [SMILES: Cc1nc(SCC(=O)Nc2ccccc2C#N)c2c(-c3cccs3)csc2n1] | No | No | Not tested |
| 20B | Enamine, Z24279508 | 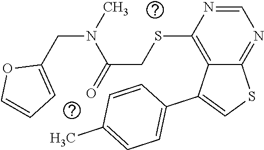 [SMILES: Cc1ccc(-c2csc3ncnc(SCC(=O)N(C)Cc4ccco4)c23)cc1] | No | Yes | Lower IC₅₀ ratio |
| 21B | Enamine, Z16638047 | 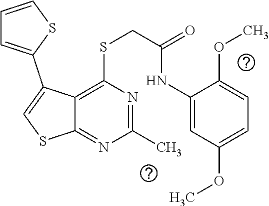 [SMILES: COc1ccc(OC)c(NC(=O)CSc2nc(C)nc3scc(-c4cccs4)c23)c1] | No | No | Not tested |
| 22B | Enamine, Z16638345 | 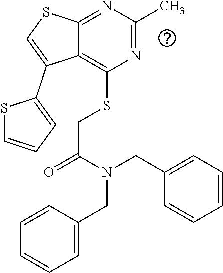 [SMILES: Cc1nc(SCC(=O)N(Cc2ccccc2)Cc2ccccc2)c2c(-c3cccs3)csc2n1] | No | No | Not tested |
| 23B | Enamine, Z128131610 | 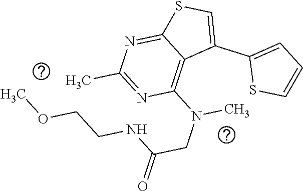 [SMILES: COCCNC(=O)CN(C)c1nc(C)nc2scc(-c3cccs3)c12] | No | Yes | Lower IC₅₀ ratio |
| 24B | Enamine, Z2239074082 | 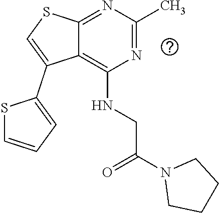 [SMILES: Cc1nc(NCC(=O)N2CCCC2)c2c(-c3cccs3)csc2n1] | No | Yes | Lower IC₅₀ ratio |
| 25B | Enamine, Z2239055645 | 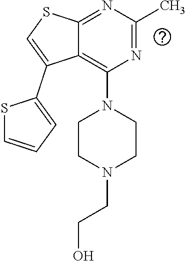 [SMILES: Cc1nc(N2CCN(CCO)CC2)c2c(-c3cccs3)csc2n1] | No | No | Not tested |
| 26B | Enamine, Z16637989 | 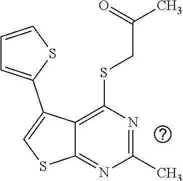 [SMILES: CC(=O)CSc1nc(C)nc2scc(-c3cccs3)c12] | No | No | Not tested |
| 27B | Enamine, Z16638153 | 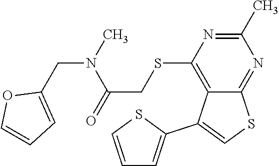 [SMILES: Cc1nc(SCC(=O)N(C)Cc2ccco2)c2c(-c3cccs3)csc2n1] | No | No | Not tested |
| 28B | Enamine, Z16637507 | 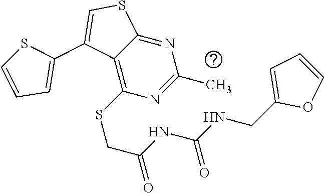 [SMILES: Cc1nc(SCC(=O)NC(=O)NCc2ccco2)c2c(-c3cccs3)csc2n1] | No | No | Not tested |
| 29B | Enamine, Z16637668 | 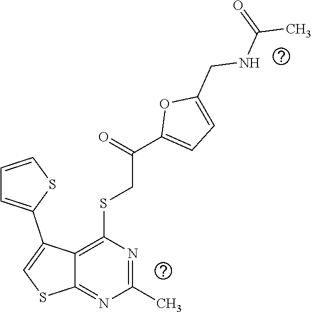 [SMILES: CC(=O)NCc1ccc(C(=O)CSc2nc(C)nc3scc(-c4cccs4)c23)o1] | Yes | No | Not tested |
| 30B | Enamine, Z397859556 | 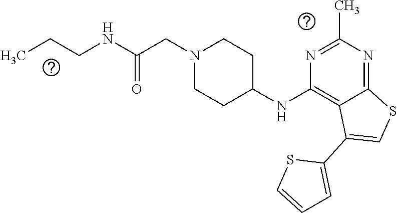 [SMILES: CCCNC(=O)CN1CCC(Nc2nc(C)nc3scc(-c4cccs4)c23)CC1] | No | No | Not tested |
| 31B | Enamine, Z16637150 | 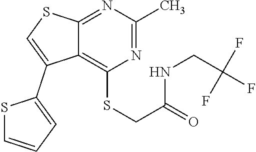 [SMILES: Cc1nc(SCC(=O)NCC(F)(F)F)c2c(-c3cccs3)csc2n1] | No | No | Not tested |
| 32B | Enamine, Z16637297 | 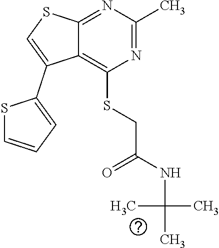 [SMILES: Cc1nc(SCC(=O)NC(C)(C)C)c2c(-c3cccs3)csc2n1] | Yes | Yes | Lower IC₅₀ ratio |
| 33B | Enamine, Z66006063 | 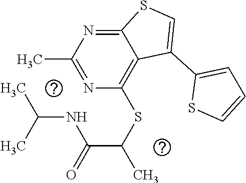 [SMILES: Cc1nc(SC(C)C(=O)NC(C)C)c2c(-c3cccs3)csc2n1] | No | Yes | Lower IC₅₀ ratio |
| 34B | Enamine, Z16637674 | 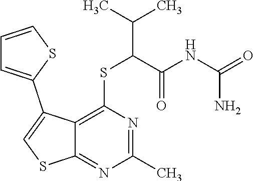 [SMILES: Cc1nc(SC(C(=O)NC(N)=O)C(C)C)c2c(-c3cccs3)csc2n1] | No | No | Not tested |
| 35B | Enamine, Z31245547 | 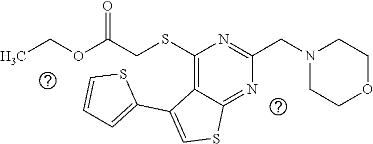 [SMILES: CCOC(=O)CSc1nc(CN2CCOCC2)nc2scc(-c3cccs3)c12] | No | No | Not tested |
| 36B | Enamine, Z16638028 | 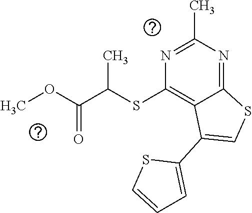 [SMILES: COC(=O)C(C)Sc1nc(C)nc2scc(-c3cccs3)c12] | No | No | Not tested |
| 37B | Enamine, Z31124373 | 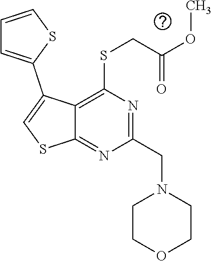 [SMILES: COC(=O)CSc1nc(CN2CCOCC2)nc2scc(-c3cccs3)c12] | No | No | Not tested |
| 38B | Enamine, Z16637475 | 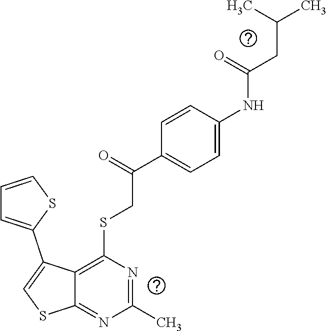 [SMILES: Cc1nc(SCC(=O)c2ccc(NC(=O)CC(C)C)cc2)c2c(-c3cccs3)csc2n1] | No | No | Not tested |
| 39B | Enamine, Z448175334 | 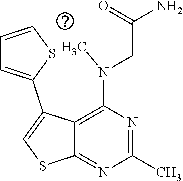 [SMILES: Cc1nc(N(C)CC(N)=O)c2c(-c3cccs3)csc2n1] | No | No | Not tested |
| 40B | Enamine, Z20234968 | 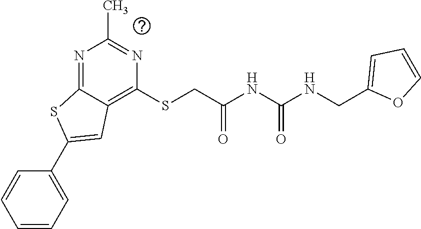 [SMILES: Cc1nc(SCC(=O)NC(=O)NCc2ccco2)c2cc(-c3ccccc3)sc2n1] | No | Yes | Lower IC₅₀ ratio |
| 41B | Enamine, Z16638133 | 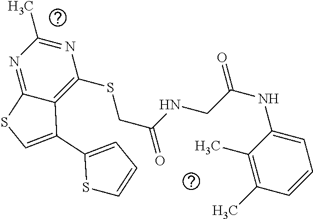 [SMILES: Cc1nc(SCC(=O)NCC(=O)Nc2cccc(C)c2C)c2c(-c3cccs3)csc2n1] | No | No | Not tested |
| 42B | Enamine, Z2239071382 | 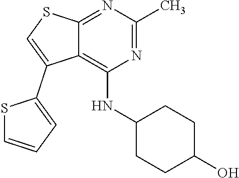 [SMILES: Cc1nc(NC2CCC(O)CC2)c2c(-c3cccs3)csc2n1] | No | No | Not tested |
| 43B | Enamine, Z16637020 | 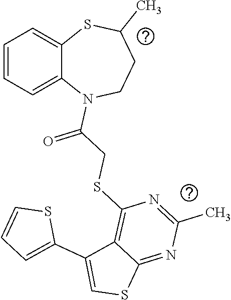 [SMILES: Cc1nc(SCC(=O)N2CCC(C)Sc3ccccc32)c2c(-c3cccs3)csc2n1] | No | No | Not tested |
| 44B | Enamine, Z16637285 | 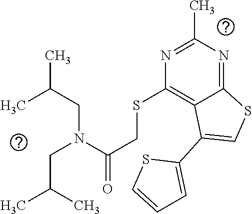 [SMILES: Cc1nc(SCC(=O)N(CC(C)C)CC(C)C)c2c(-c3cccs3)csc2n1] | No | Yes | Lower IC₅₀ ratio |
| 45B | Enamine, Z16638041 | 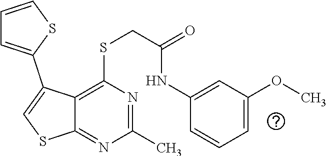 [SMILES: COc1cccc(NC(=O)CSc2nc(C)nc3scc(-c4cccs4)c23)c1] | No | Yes | Higher IC₅₀ ratio, but false-positive |
| 46B | Enamine, Z786277332 | 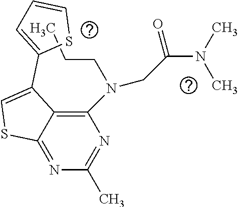 | No | Yes | Lower IC₅₀ ratio |
| 47B | Enamine, Z354308096 | 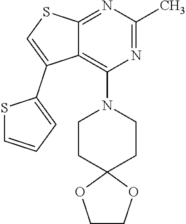 [SMILES: Cc1nc(N2CCC3(CC2)OCCO3)c2c(-c3cccs3)csc2n1] | No | Yes | Lower IC₅₀ ratio |
| 48B | Enamine, Z2239076139 | 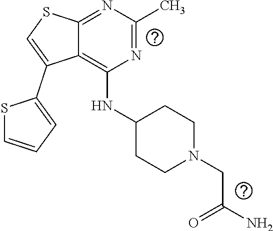 [SMILES: Cc1nc(NC2CCN(CC(N)=O)CC2)c2c(-c3cccs3)csc2n1] | No | Yes | Lower IC₅₀ ratio |
| 49B | Enamine, Z16637531 | 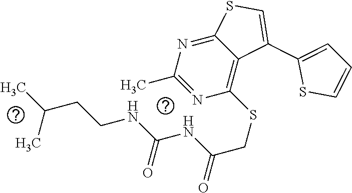 [SMILES: Cc1nc(SCC(=O)NC(=O)NCCC(C)C)c2c(-c3cccs3)csc2n1] | No | Yes | Lower IC₅₀ ratio |
| 1C | Enamine, Z64573976 | 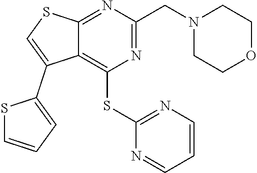 [SMILES: c1cnc(Sc2nc(CN3CCOCC3)nc3scc(-c4cccs4)c23)nc1] | No | Yes | Lower IC₅₀ ratio |
| 2C | Enamine, Z16637611 | 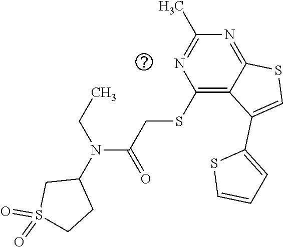 [SMILES: CCN(C(=O)CSc1nc(C)nc2scc(-c3cccs3)c12)C1CCS(=O)(=O)C1] | Yes | Yes | Lower IC₅₀ ratio |
| 3C | Enamine, Z31241653 | 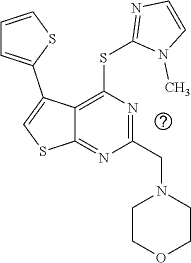 [SMILES: Cn1ccnc1Sc1nc(CN2CCOCC2)nc2scc(-c3cccs3)c12] | No | No | Not tested |
| 4C | Enamine, Z128146694 | 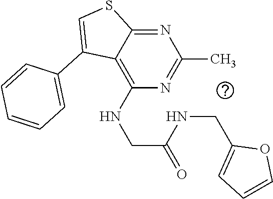 [SMILES: Cc1nc(NCC(=O)NCc2ccco2)c2c(-c3ccccc3)csc2n1] | No | Yes | Higher IC₅₀ ratio and confirmed |
| 5C | Enamine, Z16638030 | 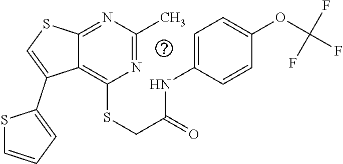 [SMILES: Cc1nc(SCC(=O)Nc2ccc(OC(F)(F)F)cc2)c2c(-c3cccs3)csc2n1] | No | Yes | Lower IC₅₀ ratio |
| 6C | Enamine, Z31109633 | 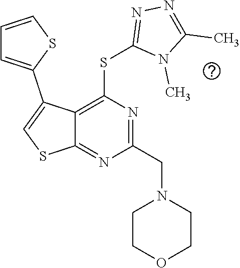 [SMILES: Cc1nnc(Sc2nc(CN3CCOCC3)nc3scc(-c4cccs4)c23)n1C] | No | Yes | Lower IC₅₀ ratio |
| 7C | Enamine, Z17142867 | 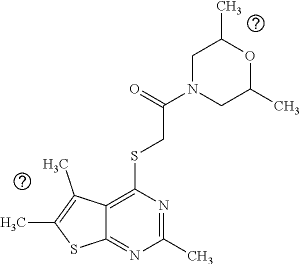 | No | Yes | Higher IC₅₀ ratio, but false-positive |
| 8C | Enamine, Z128193812 | 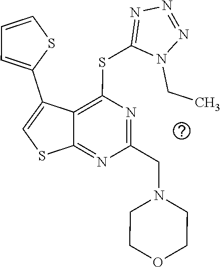 [SMILES: CCn1nnnc1Sc1nc(CN2CCOCC2)nc2scc(-c3cccs3)c12] | No | Yes | Lower IC₅₀ ratio |
| 9C | Enamine, Z24279303 | 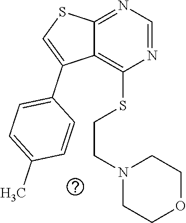 [SMILES: Cc1ccc(-c2csc3ncnc(SCCN4CCOCC4)c23)cc1] | No | Yes | Lower IC₅₀ ratio |
| 10C | Enamine, Z16636904 | 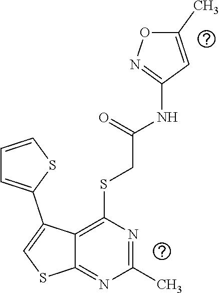 [SMILES: Cc1nc(SCC(=O)Nc2cc(C)on2)c2c(-c3cccs3)csc2n1] | Yes | Yes | Lower IC₅₀ ratio |
| 11C | Enamine, Z16638360 | 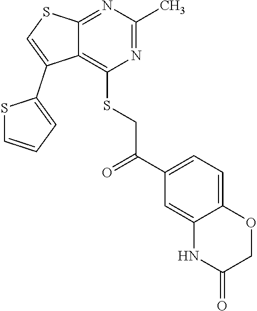 [SMILES: Cc1nc(SCC(=O)c2ccc3c(c2)NC(=O)CO3)c2c(-c3cccs3)csc2n1] | Yes | Yes | Higher IC₅₀ ratio and confirmed |
| 12C | Enamine, Z25464129 | 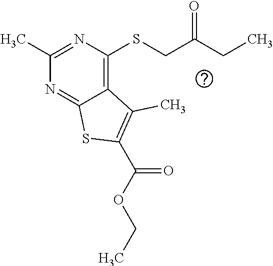 [SMILES: CCOC(=O)c1sc2nc(C)nc(SCC(=O)CC)c2c1C] | No | No | Not tested |
| 13C | Enamine, Z31206475 | 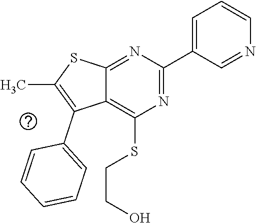 [SMILES: Cc1sc2nc(-c3cccnc3)nc(SCCO)c2c1-c1ccccc1] | No | Yes | Lower IC₅₀ ratio |
| 14C | Enamine, Z20233718 | 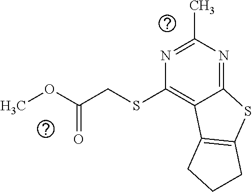 [SMILES: COC(=O)CSc1nc(C)nc2sc3c(c12)CCC3] | No | No | Not tested |
| 15C | Enamine, Z2239056480 | 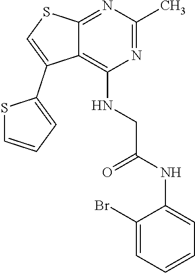 [SMILES: Cc1nc(NCC(=O)Nc2ccccc2Br)c2c(-c3cccs3)csc2n1] | No | Yes | Higher IC₅₀ ratio and confirmed |
| 16C | Enamine, Z200146872 | 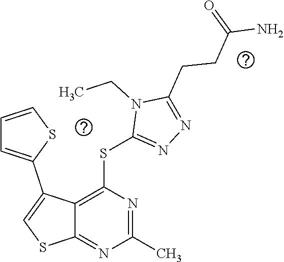 [SMILES: CCn1c(CCC(N)=O)nnc1Sc1nc(C)nc2scc(-c3cccs3)c12] | Yes | Yes | Lower IC₅₀ ratio |
| 17C | Enamine, Z31206396 | 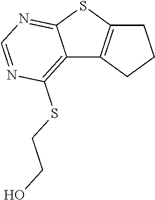 [SMILES: OCCSc1ncnc2sc3c(c12)CCC3] | No | No | Not tested |
| 18C | Enamine, Z31206484 | 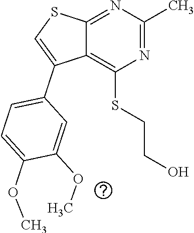 [SMILES: COc1ccc(-c2csc3nc(C)nc(SCCO)c23)cc1OC] | No | No | Not tested |
| 19C | Enamine, Z16638378 | 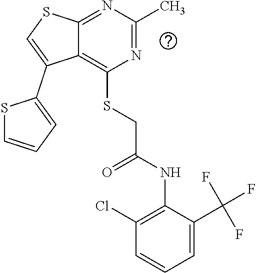 [SMILES: Cc1nc(SCC(=O)Nc2c(Cl)cccc2C(F)(F)F)c2c(-c3cccs3)csc2n1] | No | No | Not tested |
| 20C | Enamine, Z31085357 | 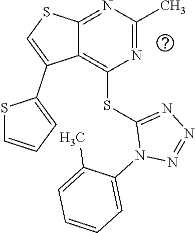 [SMILES: Cc1nc(Sc2nnnn2-c2ccccc2C)c2c(-c3cccs3)csc2n1] | No | Yes | Lower IC₅₀ ratio |
| 21C | Enamine, Z103173086 | 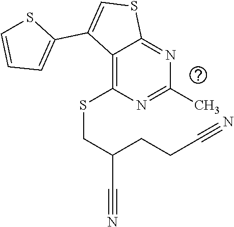 [SMILES: Cc1nc(SCC(C#N)CCC#N)c2c(-c3cccs3)csc2n1] | No | No | Not tested |
| 22C | Enamine, Z31206398 | 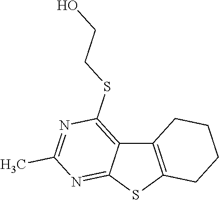 [SMILES: Cc1nc(SCCO)c2c3c(sc2n1)CCCC3] | No | No | Not tested |
| 23C | Enamine, Z31206498 | 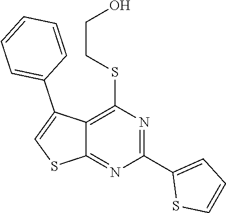 [SMILES: OCCSc1nc(-c2cccs2)nc2scc(-c3ccccc3)c12] | No | Yes | Lower IC₅₀ ratio |
| 24C | Enamine, Z31206372 | 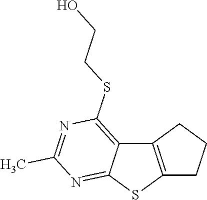 [SMILES: Cc1nc(SCCO)c2c3c(sc2n1)CCC3] | No | No | Not tested |
| 25C | Enamine, Z31147483 | 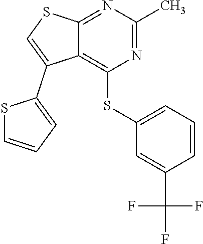 [SMILES: Cc1nc(Sc2cccc(C(F)(F)F)c2)c2c(-c3cccs3)csc2n1] | No | Yes | Lower IC₅₀ ratio |
| 26C | Enamine, Z317301078 | 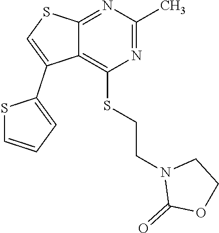 [SMILES: Cc1nc(SCCN2CCOC2=O)c2c(-c3cccs3)csc2n1] | Yes | No | Not tested |
| 27C | Enamine, Z31206377 | 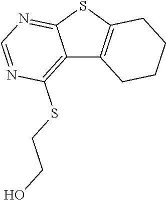 [SMILES: OCCSc1ncnc2sc3c(c12)CCCC3] | No | No | Not tested |
| 28C | Enamine, Z20235483 | 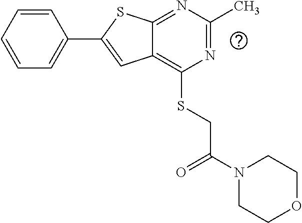 [SMILES: Cc1nc(SCC(=O)N2CCOCC2)c2cc(-c3ccccc3)sc2n1] | No | No | Not tested |
| 29C | Enamine, Z31177327 | 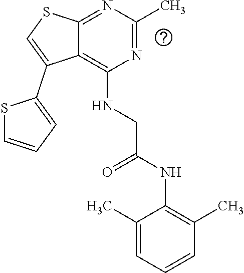 [SMILES: Cc1nc(NCC(=O)Nc2c(C)cccc2C)c2c(-c3cccs3)csc2n1] | No | No | Not tested |
| 30C | Enamine, Z31206400 | 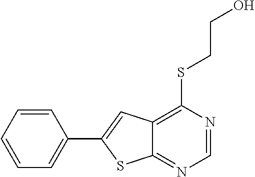 [SMILES: OCCSc1ncnc2sc(-c3ccccc3)cc12] | No | No | Not tested |
| 31C | Enamine, Z20231326 | 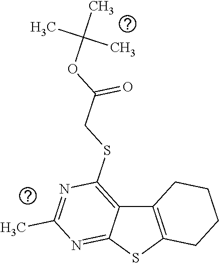 [SMILES: Cc1nc(SCC(=O)OC(C)(C)C)c2c3c(sc2n1)CCCC3] | No | No | Not tested |
| 32C | Enamine, Z16636927 | 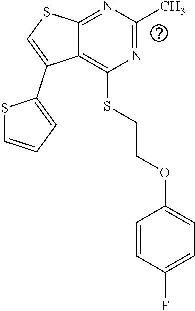 [SMILES: Cc1nc(SCCOc2ccc(F)cc2)c2c(-c3cccs3)csc2n1] | No | No | Not tested |
| 33C | Enamine, Z1270086424 | 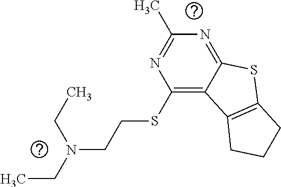 [SMILES: CCN(CC)CCSc1nc(C)nc2sc3c(c12)CCC3] | No | Yes | Lower IC₅₀ ratio |
| 34C | Enamine, Z199944968 | 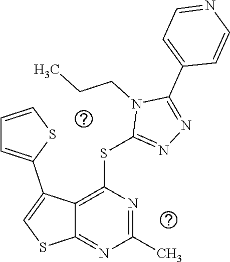 [SMILES: CCCn1c(Sc2nc(C)nc3scc(-c4cccs4)c23)nnc1-c1ccncc1] | No | Yes | Lower IC₅₀ ratio |
| 35C | Enamine, Z229254086 | 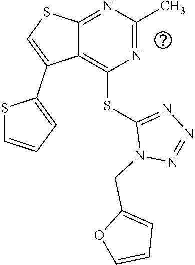 [SMILES: Cc1nc(Sc2nnnn2Cc2ccco2)c2c(-c3cccs3)csc2n1] | No | Yes | Lower IC₅₀ ratio |
| 36C | Enamine, Z1270086356 | 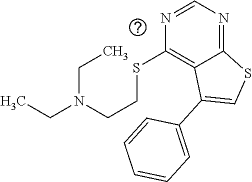 [SMILES: CCN(CC)CCSc1ncnc2scc(-c3ccccc3)c12] | No | Yes | Lower IC₅₀ ratio |
| 37C | Enamine, Z215012068 | 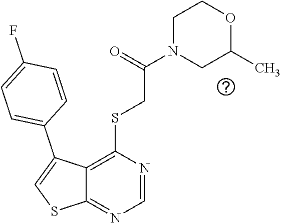 [SMILES: CC1CN(C(=O)CSc2ncnc3scc(-c4ccc(F)cc4)c23)CCO1] | No | No | Not tested |
| 38C | Enamine, Z199967676 | 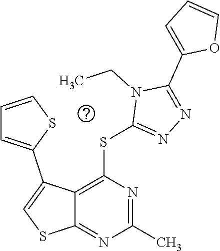 [SMILES: CCn1c(Sc2nc(C)nc3scc(-c4cccs4)c23)nnc1-c1ccco1] | No | No | Not tested |
| 39C | Enamine, Z246512462 | 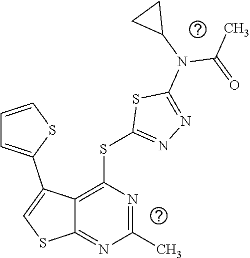 [SMILES: CC(=O)N(c1nnc(Sc2nc(C)nc3scc(-c4cccs4)c23)s1)C1CC1] | No | No | Not tested |
| 40C | Enamine, Z1478446288 | 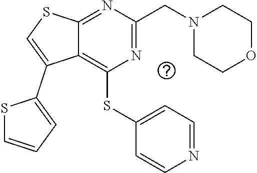 [SMILES: c1csc(-c2csc3nc(CN4CCOCC4)nc(Sc4ccncc4)c23)c1] | No | Yes | Lower IC₅₀ ratio |
| 41C | Enamine, Z20235408 | 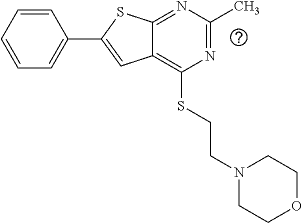 [SMILES: Cc1nc(SCCN2CCOCC2)c2cc(-c3ccccc3)sc2n1] | No | Yes | Lower IC₅₀ ratio |
| 42C | Enamine, Z1526785534 | 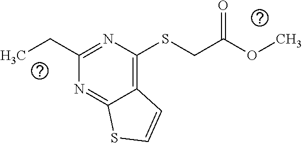 [SMILES: CCc1nc(SCC(=O)OC)c2ccsc2n1] | No | No | Lower IC₅₀ ratio |
| 43C | Enamine, Z199784892 | 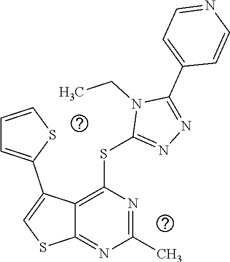 [SMILES: CCn1c(Sc2nc(C)nc3scc(-c4cccs4)c23)nnc1-c1ccncc1] | Yes | Yes | Not tested |
| 44C | Enamine, Z128124404 | 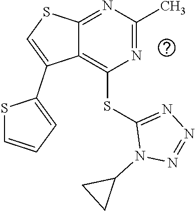 [SMILES: Cc1nc(Sc2nnnn2C2CC2)c2c(-c3cccs3)csc2n1] | No | Yes | Lower IC₅₀ ratio |
| 45C | Enamine, Z424820732 | 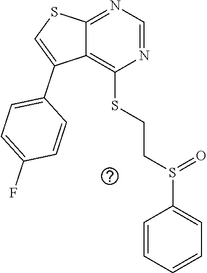 [SMILES: O=S(CCSc1ncnc2scc(-c3ccc(F)cc3)c12)c1ccccc1] | No | Yes | Lower IC₅₀ ratio |
| 46C | Enamine, Z200177070 | 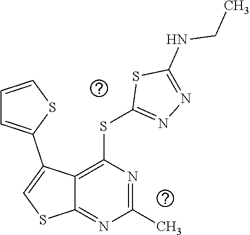 [SMILES: CCNc1nnc(Sc2nc(C)nc3scc(-c4cccs4)c23)s1] | No | No | Not tested |
| 47C | Enamine, Z17142572 | 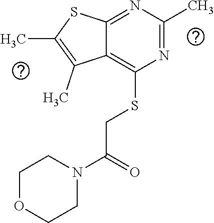 [SMILES: Cc1nc(SCC(=O)N2CCOCC2)c2c(C)c(C)sc2n1] | No | No | Not tested |
| 48C | Enamine, Z2237508497 | 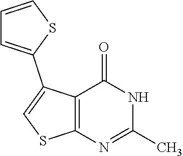 [SMILES: Cc1nc2scc(-c3cccs3)c2c(=O)[nH]1] | No | No | Not tested |
| 49C | Enamine, Z20233712 | 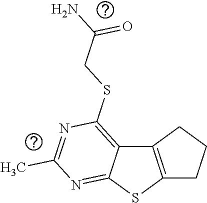 [SMILES: Cc1nc(SCC(N)=O)c2c3c(sc2n1)CCC3] | No | No | Not tested |
| 50C | Enamine, Z2241107700 | 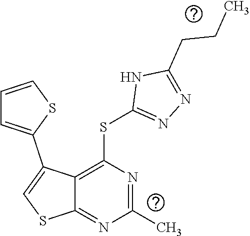 [SMILES: CCCc1nnc(Sc2nc(C)nc3scc(-c4cccs4)c23)[nH]1] | Yes | Yes | Lower IC₅₀ ratio |
| 51C | Enamine, Z56770504 | 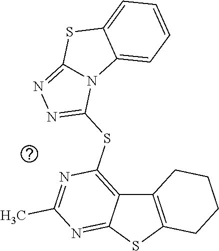 [SMILES: Cc1nc(Sc2nnc3sc4ccccc4n23)c2c3c(sc2n1)CCCC3] | No | No | Not tested |
| 52C | Enamine, Z20232029 | 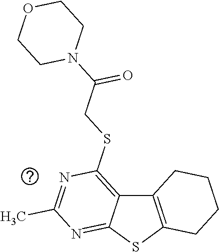 [SMILES: Cc1nc(SCC(=O)N2CCOCC2)c2c3c(sc2n1)CCCC3] | No | No | Not tested |
| 53C | Enamine, Z25603837 | 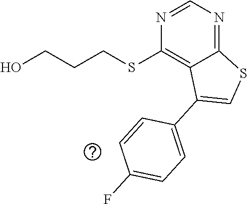 [SMILES: OCCCSc1ncnc2scc(-c3ccc(F)cc3)c12] | No | No | Not tested |
| 54C | Enamine, Z31120255 | 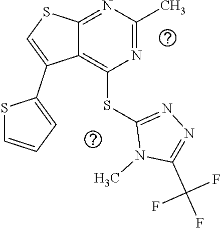 [SMILES: Cc1nc(Sc2nnc(C(F)(F)F)n2C)c2c(-c3cccs3)csc2n1] | Yes | Yes | Lower IC₅₀ ratio |
| 55C | Enamine, Z20231985 | 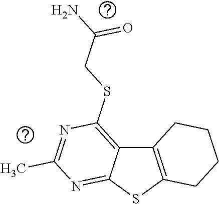 [SMILES: Cc1nc(SCC(N)=O)c2c3c(sc2n1)CCCC3] | No | No | Not tested |
| 56C | Enamine, Z31206432 | 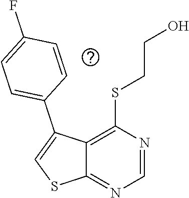 [SMILES: OCCSc1ncnc2scc(-c3ccc(F)cc3)c12] | No | No | Not tested |
| 57C | Enamine, Z31206412 | 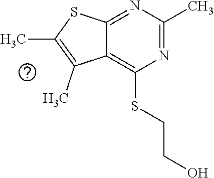 [SMILES: Cc1nc(SCCO)c2c(C)c(C)sc2n1] | No | No | Not tested |
| 58C | Enamine, Z200194736 | 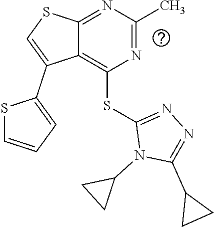 [SMILES: Cc1nc(Sc2nnc(C3CC3)n2C2CC2)c2c(-c3cccs3)csc2n1] | No | No | Not tested |
| 59C | Enamine, Z25464123 | 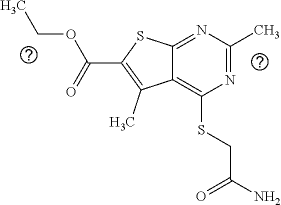 [SMILES: CCOC(=O)c1sc2nc(C)nc(SCC(N)=O)c2c1C] | No | No | Not tested |
| 60C | Enamine, Z20245845 | 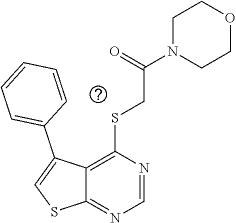 [SMILES: O=C(CSc1ncnc2scc(-c3ccccc3)c12)N1CCOCC1] | No | No | Not tested |
| 61C | Enamine, Z17142544 | 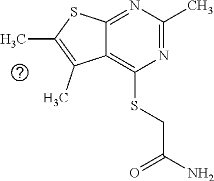 [SMILES: Cc1nc(SCC(N)=O)c2c(C)c(C)sc2n1] | No | No | Not tested |
| 62C | Enamine, Z2241097168 | 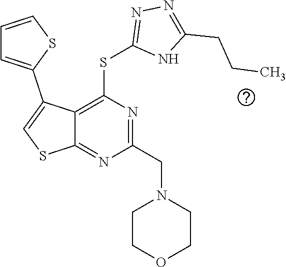 [SMILES: CCCc1nnc(Sc2nc(CN3CCOCC3)nc3scc(-c4cccs4)c23)[nH]1] | No | Yes | Lower IC₅₀ ratio |
| 63C | Enamine, Z199817466 | 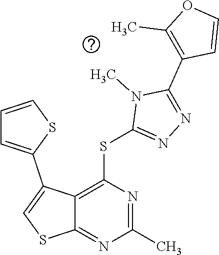 [SMILES: Cc1nc(Sc2nnc(-c3ccoc3C)n2C)c2c(-c3cccs3)csc2n1] | No | No | Not tested |
| 64C | Enamine, Z64565307 | 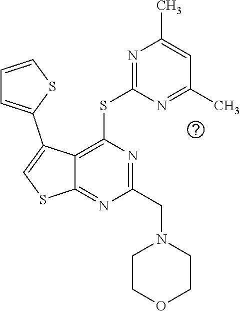 [SMILES: Cc1cc(C)nc(Sc2nc(CN3CCOCC3)nc3scc(-c4cccs4)c23)n1] | No | Yes | Lower IC₅₀ ratio |
| 65C | Enamine, Z20233756 | 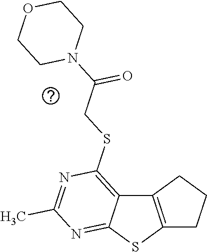 [SMILES: Cc1nc(SCC(=O)N2CCOCC2)c2c3c(sc2n1)CCC3] | No | No | Not tested |
| 66C | Enamine, Z86389283 | 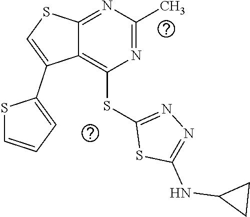 [SMILES: Cc1nc(Sc2nnc(NC3CC3)s2)c2c(-c3cccs3)csc2n1] | No | No | Not tested |
| 67C | Enamine, Z86409302 | 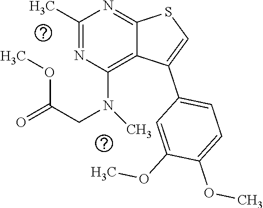 [SMILES: COC(=O)CN(C)c1nc(C)nc2scc(-c3ccc(OC)c(OC)c3)c12] | No | Yes | Lower IC₅₀ ratio |
| 68C | Life Chemicals, F5481-2262 | 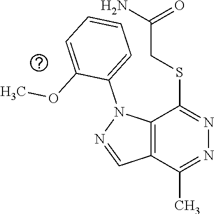 [SMILES: COc1ccccc1-n1ncc2c(C)nnc(SCC(N)=O)c21] | No | No | Not tested |
| 69C | Life Chemicals, F5481-0004 | 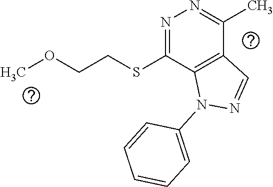 | No | No | Not tested |
| 70C | Life Chemicals, F5481-0006 | 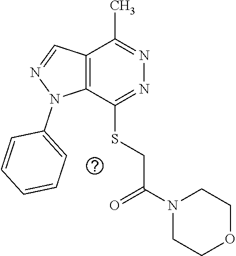 [SMILES: Cc1nnc(SCC(=O)N2CCOCC2)c2c1cnn2-c1ccccc1] | No | No | Not tested |
| 71C | Life Chemicals, F5481-0025 | 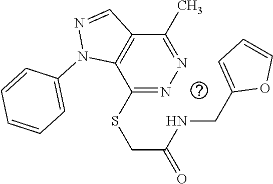 [SMILES: Cc1nnc(SCC(=O)NCc2ccco2)c2c1cnn2-c1ccccc1] | No | No | Not tested |
| 72C | Life Chemicals, F5481-0033 | 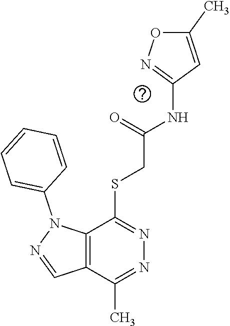 [SMILES: Cc1cc(NC(=O)CSc2nnc(C)c3cnn(-c4ccccc4)c23)no1] | No | No | Not tested |
| 73C | Life Chemicals, F5481-0091 | 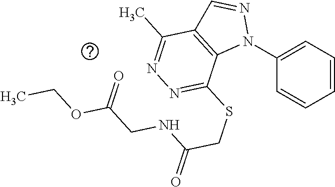 [SMILES: CCOC(=O)CNC(=O)CSc1nnc(C)c2cnn(-c3ccccc3)c12] | No | No | Not tested |
| 74C | Life Chemicals, F5481-0133 | 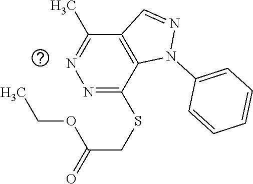 [SMILES: CCOC(=O)CSc1nnc(C)c2cnn(-c3ccccc3)c12] | No | No | Not tested |
| 75C | Life Chemicals, F5481-0150 | 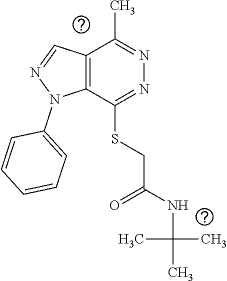 [SMILES: Cc1nnc(SCC(=O)NC(C)(C)C)c2c1cnn2-c1ccccc1] | No | No | Not tested |
| 76C | Life Chemicals, F5481-2225 | 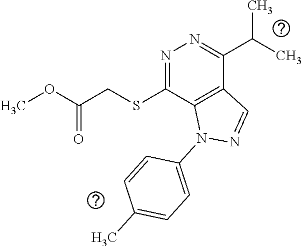 [SMILES: COC(=O)CSc1nnc(C(C)C)c2cnn(-c3ccc(C)cc3)c12] | No | Yes | Lower IC₅₀ ratio |
| 77C | Life Chemicals, F5481-2260 | 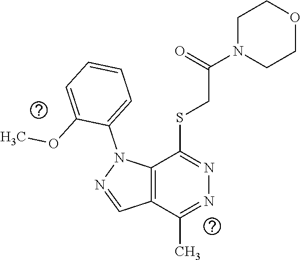 [SMILES: COc1ccccc1-n1ncc2c(C)nnc(SCC(=O)N3CCOCC3)c21] | No | No | Not tested |
| 78C | Enamine, Z56882048 | 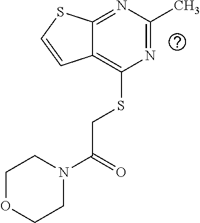 [SMILES: Cc1nc(SCC(=O)N2CCOCC2)c2ccsc2n1] | No | No | Not tested |
| 79C | LabNetwork, 500194-47-8 | 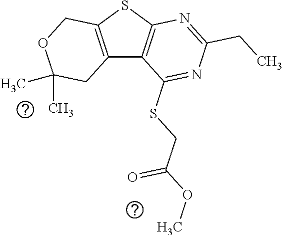 [SMILES: CCc1nc(SCC(=O)OC)c2c3c(sc2n1)COC(C)(C)C3] | No | No | Not tested |
| 80C | LabNetwork, 844449-63-4 | 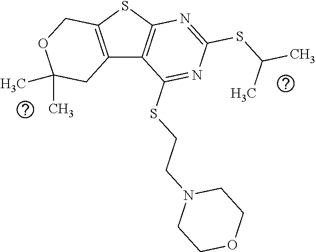 [SMILES: CC(C)Sc1nc(SCCN2CCOCC2)c2c3c(sc2n1)COC(C)(C)C3] | No | No | Not tested |
| 81C | LabNetwork, 844652-80-8 | 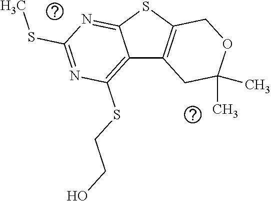 | No | Yes | Lower IC₅₀ ratio |
| 82C | LabNetwork, 332870-43-6 | 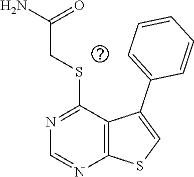 [SMILES: NC(=O)CSc1ncnc2scc(-c3ccccc3)c12] | No | No | Not tested |
| 83C | Uorsy, PB1478272140 | 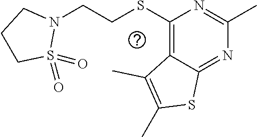 [SMILES: Cc1nc(SCCN2CCCS2(=O)=O)c2c(C)c(C)sc2n1] | No | No | Not tested |
| 84C | Uorsy, PB20304241 | 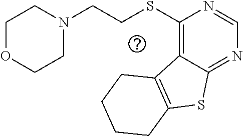 [SMILES: c1nc(SCCN2CCOCC2)c2c3c(sc2n1)CCCC3] | No | No | Not tested |
| 85C | Uorsy, PB31316924 | 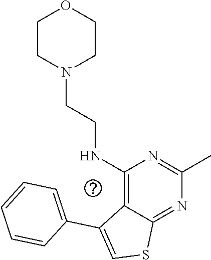 [SMILES: Cc1nc(NCCN2CCOCC2)c2c(-c3ccccc3)csc2n1] | No | Yes | Higher IC₅₀ ratio, but false-positive |
| 86C | Uorsy, PB15764662 | 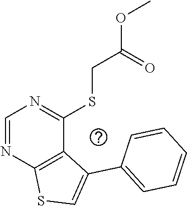 [SMILES: COC(=O)CSc1ncnc2scc(-c3ccccc3)c12] | No | No | Not tested |
| 87C | Enamine, Z3397472468 | 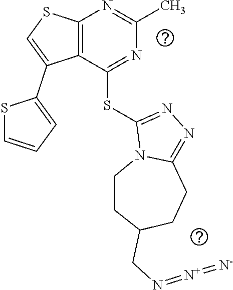 [SMILES: Cc1nc(Sc2nnc3n2CCC(CN=[N+]=[N-])CC3)c2c(-c3cccs3)csc2n1] | Yes | Yes | Lower IC₅₀ ratio |

WBC-A and its structural analogs are listed with their corresponding vendor, ID-code and molecular structure. A distinction is made between compounds that could or could not rescue tps1Δ growth on 2.5 mM glucose. In addition, compounds selected from the primary A549 growth inhibitory screen and compounds with a higher IC50 ratio (10 mM glucose: 1 mM glucose) compared to WBC-A are indicated.

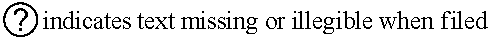

### REFERENCES

1. Diaz-Ruiz R, Uribe-Carvajal S, Devin A, Rigoulet M. Tumor cell energy metabolism and its common features with yeast metabolism. Biochim Biophys Acta 1796, 252-265 (2009).
2. Vander Heiden M G, Cantley L C, Thompson C B. Understanding the Warburg effect: the metabolic requirements of cell proliferation. Science 324, 1029-1033 (2009).
3. Warburg O. On the origin of cancer cells. Science 123, 309-314 (1956).
4. Postma E, Verduyn C, Scheffers W A, Van Dijken J P. Enzymic analysis of the crabtree effect in glucose-limited chemostat cultures of Saccharomyces cerevisiae. Appl Environ Microbiol 55, 468-477 (1989).
5. Upadhyay M, Samal J, Kandpal M, Singh O V, Vivekanandan P. The Warburg effect: insights from the past decade. Pharmacol Ther 137, 318-330 (2013).
6. Czernin J, Phelps M E. Positron emission tomography scanning: current and future applications. Annu Rev Med 53, 89-112 (2002).
7. Tanner L B, et al. Four Key Steps Control Glycolytic Flux in Mammalian Cells. Cell Syst 7, 49-62 e48 (2018).
8. Rodriguez-Enriquez S, Marin-Hernandez A, Gallardo-Perez J C, Moreno-Sanchez R. Kinetics of transport and phosphorylation of glucose in cancer cells. J Cell Physiol 221, 552-559 (2009).
9. Mathupala S P, Rempel A, Pedersen P L. Aberrant glycolytic metabolism of cancer cells: a remarkable coordination of genetic, transcriptional, post-translational, and mutational events that lead to a critical role for type II hexokinase. J Bioenerg Biomembr 29, 339-343 (1997).
10. Marin-Hernandez A, et al. Determining and understanding the control of glycolysis in fast-growth tumor cells. Flux control by an over-expressed but strongly product-inhibited hexokinase. FEBS J 273, 1975-1988 (2006).
11. Younes M, Lechago L V, Somoano J R, Mosharaf M, Lechago J. Wide expression of the human erythrocyte glucose transporter Glut1 in human cancers. Cancer Res 56, 1164-1167 (1996).
12. Macheda M L, Rogers S, Best J D. Molecular and cellular regulation of glucose transporter (GLUT) proteins in cancer. J Cell Physiol 202, 654-662 (2005).
13. Johansson T, Berrez J M, Nelson B D. Evidence that transcription of the hexokinase gene is increased in a rapidly growing rat hepatoma. Biochem Biophys Res Commun 133, 608-613 (1985).
14. Rempel A, Mathupala S P, Griffin C A, Hawkins A L, Pedersen P L. Glucose catabolism in cancer cells: amplification of the gene encoding type II hexokinase. Cancer Res 56, 2468-2471 (1996).
15. Yamamoto T, et al. Over-expression of facilitative glucose transporter genes in human cancer. Biochem Biophys Res Commun 170, 223-230 (1990).
16. Kim J W, Dang C V. Cancer's molecular sweet tooth and the Warburg effect. Cancer Res 66, 8927-8930 (2006).
17. Chen X, Qian Y, Wu S. The Warburg effect: evolving interpretations of an established concept. Free Radic Biol Med 79, 253-263 (2015).
18. Pelicano H, Martin D S, Xu R H, Huang P. Glycolysis inhibition for anticancer treatment. Oncogene 25, 4633-4646 (2006).
19. Fendt S M, Sauer U. Transcriptional regulation of respiration in yeast metabolizing differently repressive carbon substrates. BMC Syst Biol 4, 12 (2010).
20. van Dijken J P, Weusthuis R A, Pronk J T. Kinetics of growth and sugar consumption in yeasts. Antonie Van Leeuwenhoek 63, 343-352 (1993).
21. Kummel A, et al. Differential glucose repression in common yeast strains in response to HXK2 deletion. FEMS Yeast Res 10, 322-332 (2010).
22. Conrad M, Schothorst J, Kankipati H N, Van Zeebroeck G, Rubio-Texeira M, Thevelein J M. Nutrient sensing and signaling in the yeast Saccharomyces cerevisiae. FEMS Microbiol Rev 38, 254-299 (2014).
23. Peeters K, et al. Fructose-1,6-bisphosphate couples glycolytic flux to activation of Ras. Nat Commun 8, 922 (2017).
24. Boles E, Hollenberg C P. The molecular genetics of hexose transport in yeasts. FEMS Microbiol Rev 21, 85-111 (1997).
25. Mueckler M, Thorens B. The SLC2 (GLUT) family of membrane transporters. Mol Aspects Med 34, 121-138 (2013).
26. Hruz P W, Mueckler M M. Structural analysis of the GLUT1 facilitative glucose transporter (review). Mol MembrBiol 18, 183-193 (2001).
27. Deng D, Yan N. GLUT, SGLT, and SWEET: Structural and mechanistic investigations of the glucose transporters. Protein Sci 25, 546-558 (2016).
28. Carruthers A, Helgerson A L. The human erythrocyte sugar transporter is also a nucleotide binding protein. Biochemistry 28, 8337-8346 (1989).
29. Blodgett D M, De Zutter J K, Levine K B, Karim P, Carruthers A. Structural basis of GLUT1 inhibition by cytoplasmic ATP. J Gen Physiol 130, 157-168 (2007).
30. Levine K B, Cloherty E K, Hamill S, Carruthers A. Molecular determinants of sugar transport regulation by ATP. Biochemistry 41, 12629-12638 (2002).
31. Cloherty E K, Levine K B, Graybill C, Carruthers A. Cooperative nucleotide binding to the human erythrocyte sugar transporter. Biochemistry 41, 12639-12651 (2002).
32. Levine K B, Hamill S, Cloherty E K, Carruthers A. Alanine scanning mutagenesis ofthe human erythrocyte glucose transporter putative ATP binding domain. Blood Cells Mol Dis 27, 139-142 (2001).
33. Mohan S, Sheena A, Poulose N, Anilkumar G. Molecular dynamics simulation studies of GLUT4: substrate-free and substrate-induced dynamics and ATP-mediated glucose transport inhibition. PLoS One 5, e14217 (2010).
34. Mohan S S, Perry J J, Poulose N, Nair B G, Anilkumar G. Homology modeling of GLUT4, an insulin regulated facilitated glucose transporter and docking studies with ATP and its inhibitors. J Biomol Struct Dyn 26, 455-464 (2009).
35. Granchi C, Fortunato S, Minutolo F. Anticancer agents interacting with membrane glucose transporters. Medchemcomm 7, 1716-1729 (2016).
36. Cardenas M L, Cornish-Bowden A, Ureta T. Evolution and regulatory role of the hexokinases. Biochim Biophys Acta 1401, 242-264 (1998).
37. Blazquez M A, Lagunas R, Gancedo C, Gancedo J M. Trehalose-6-phosphate, a new regulator of yeast glycolysis that inhibits hexokinases. FEBS Lett 329, 51-54 (1993).
38. Gonzalez M I, Stucka R, Blazquez M A, Feldmann H, Gancedo C. Molecular cloning of CIF1, a yeast gene necessary for growth on glucose. Yeast 8, 183-192 (1992).
39. Thevelein J M, Hohmann S. Trehalose synthase: guard to the gate of glycolysis in yeast?Trends Biochem Sci 20, 3-10 (1995).
40. Van Aelst L, et al. Molecular cloning of a gene involved in glucose sensing in the yeast Saccharomyces cerevisiae. Mol Microbiol 8, 927-943 (1993).
41. Vuorio O E, Kalkkinen N, Londesborough J. Cloning of two related genes encoding the 56-kDa and 123-kDa subunits of trehalose synthase from the yeast Saccharomyces cerevisiae. Eur J Biochem 216, 849-861 (1993).
42. Neves M J, et al. Control of glucose influx into glycolysis and pleiotropic effects studied in different isogenic sets of Saccharomyces cerevisiae mutants in trehalose biosynthesis. Curr Genet 27, 110-122 (1995).
43. Hohmann S, Neves M J, de Koning W, Alijo R, Ramos J, Thevelein J M. The growth and signalling defects of the ggs1 (fdp1/byp1) deletion mutant on glucose are suppressed by a deletion of the gene encoding hexokinase PII. Curr Genet 23, 281-289 (1993).
44. Van Leemputte F, Vanthienen W, Wijnants S, Van Zeebroeck G, Thevelein J M. Aberrant 50 Intracellular pH Regulation Limiting Glyceraldehyde-3-Phosphate Dehydrogenase Activity in the Glucose-Sensitive Yeast tps1Δ Mutant. mBio 11, (2020).
45. Postma P W, Lengeler J W, Jacobson G R. Phosphoenolpyruvate:carbohydrate phosphotransferase systems of bacteria. Microbiol Rev 57, 543-594 (1993).
46. Schuddemat J, van den Broek P J, van Steveninck J. The influence of ATP on sugar uptake mediated by the constitutive glucose carrier of Saccharomyces cerevisiae. Biochim Biophys Acta 937, 81-87 (1988).
47. Jaspers H T, van Steveninck J. Transport-associated phosphorylation of 2-deoxy-D-glucose in Saccharomyces fragilis. Biochim Biophys Acta 406, 370-385 (1975).
48. van Steveninck J. Transport-associated phosphorylation of 2-deoxy-D-glucose in yeast. Biochim Biophys Acta 163, 386-394 (1968).
49. Meredith S A, Romano A H. Uptake and phosphorylation of 2-deoxy-D-glucose by wild type and respiration-deficient bakers' yeast. Biochim Biophys Acta 497, 745-759 (1977).
50. Beullens M, Thevelein J M. Investigation of transport-associated phosphorylation of sugar in yeast mutants (snf3) lacking high-affinity glucose transport and in a mutant (fdp1) showing deficient regulation of initial sugar metabolism. Current Microbiology 21, 39-46 (1990).
51. Franzusoff A, Cirillo V P. Uptake and phosphorylation of 2-deoxy-D-glucose by wild-type and single-kinase strains of Saccharomyces cerevisiae. Biochim Biophys Acta 688, 295-304 (1982).
52. Lagunas R. Sugar transport in Saccharomyces cerevisiae. FEMS Microbiol Rev 10, 229-242 (1993).
53. Smits H P, Smits G J, Postma P W, Walsh M C, van Dam K. High-affinity glucose uptake in Saccharomyces cerevisiae is not dependent on the presence of glucose-phosphorylating enzymes. Yeast 12, 439-447 (1996).
54. Wieringa T, Colen A, Bos M P, Krans H M, van Dam K. Transport-associated phosphorylation of 2-deoxyglucose in rat adipocytes. Biochim Biophys Acta 847, 8-14 (1985).
55. Saha J, Coe E L. Evidence indicating the existence of two modes of glucose uptake in Ehrlich ascites tumor cells. Biochem Biophys Res Commun 26, 441-446 (1967).
56. Daum G, Keller K, Lange K. Association of glycolytic enzymes with the cytoplasmic side of the plasma membrane of glioma cells. Biochim Biophys Acta 939, 277-281 (1988).
57. Kang Y H, Coe E L. Glucose 6-phosphate-dependent binding of hexokinase to membranes of ascites tumor cells. Biochim Biophys Acta 455, 315-321 (1976).
58. Zaid H, Talior-Volodarsky I, Antonescu C, Liu Z, Klip A. GAPDH binds GLUT4 reciprocally to hexokinase-II and regulates glucose transport activity. Biochem J 419, 475-484 (2009).
59. Granchi C, Minutolo F. Anticancer agents that counteract tumor glycolysis. ChemMedChem 7, 1318-1350 (2012).
60. Cardaci S, Desideri E, Ciriolo M R. Targeting aerobic glycolysis: 3-bromopyruvate as a promising anticancer drug. J Bioenerg Biomembr 44, 17-29 (2012).
61. Pedersen P L. 3-Bromopyruvate (3BP) a fast acting, promising, powerful, specific, and effective “small molecule” anti-cancer agent taken from labside to bedside: introduction to a special issue. J Bioenerg Biomembr 44, 1-6 (2012).
62. Liu Y, et al. A small-molecule inhibitor of glucose transporter 1 downregulates glycolysis, induces cell-cycle arrest, and inhibits cancer cell growth in vitro and in vivo. Mol Cancer Ther 11, 1672-1682 (2012).
63. Nehlin J O, Carlberg M, Ronne H. Yeast galactose permease is related to yeast and mammalian glucose transporters. Gene 85, 313-319 (1989).
64. Schmidl S, lancu C V, Choe J Y, Oreb M. Ligand Screening Systems for Human Glucose Transporters as Tools in Drug Discovery. Front Chem 6, 183 (2018).
65. Wieczorke R, Dlugai S, Krampe S, Boles E. Characterisation of mammalian GLUT glucose transporters in a heterologous yeast expression system. Cell Physiol Biochem 13, 123-134 (2003).
66. Reifenberger E, Boles E, Ciriacy M. Kinetic characterization of individual hexose transporters of Saccharomyces cerevisiae and their relation to the triggering mechanisms of glucose repression. Eur J Biochem 245, 324-333 (1997).
67. Hahn S, Maurer P, Caesar S, Schlenstedt G. Classical NLS proteins from Saccharomyces cerevisiae. J Mol Biol 379, 678-694 (2008).
68. Bisson L F, Fraenkel D G. Involvement of kinases in glucose and fructose uptake by Saccharomyces cerevisiae. Proc Natl Acad Sci USA 80, 1730-1734 (1983).
69. Carruthers A. ATP regulation of the human red cell sugar transporter. J Biol Chem 261, 11028-11037 (1986).
70. Hebert D N, Carruthers A. Direct evidence for ATP modulation of sugar transport in human erythrocyte ghosts. J Biol Chem 261, 10093-10099 (1986).
71. Lachaal M, Spangler R A, Jung C Y. Adenosine and adenosine triphosphate modulate the substrate binding affinity of glucose transporter GLUT1 in vitro. Biochim Biophys Acta 1511, 123-133 (2001).
72. Heard K S, Fidyk N, Carruthers A. ATP-dependent substrate occlusion by the human erythrocyte sugar transporter. Biochemistry 39, 3005-3014 (2000).
73. Helgerson A L, Hebert D N, Naderi S, Carruthers A. Characterization of two independent modes of action of ATP on human erythrocyte sugar transport. Biochemistry 28, 6410-6417 (1989).
74. Ovadi J. Physiological significance of metabolic channelling. J Theor Biol 152, 1-22 (1991).
75. Zhao Y, Butler E B, Tan M. Targeting cellular metabolism to improve cancer therapeutics. Cell Death Dis 4, e532 (2013).
76. Ngo H, Tortorella S M, Ververis K, Karagiannis T C. The Warburg effect: molecular aspects and therapeutic possibilities. Mol Biol Rep 42, 825-834 (2015).
77. Liu H, Hu Y P, Savaraj N, Priebe W, Lampidis T J. Hypersensitization of tumor cells to glycolytic inhibitors. Biochemistry 40, 5542-5547 (2001).
78. Shi Y, Liu S, Ahmad S, Gao Q. Targeting Key Transporters in Tumor Glycolysis as a Novel Anticancer Strategy. Curr Top Med Chem 18, 454-466 (2018).
79. Cao X, et al. Glucose uptake inhibitor sensitizes cancer cells to daunorubicin and overcomes drug resistance in hypoxia. Cancer Chemother Pharmacol 59, 495-505 (2007).
80. McBrayer S K, Cheng J C, Singhal S, Krett N L, Rosen S T, Shanmugam M. Multiple myeloma exhibits novel dependence on GLUT4, GLUT8, and GLUT11: implications for glucose transporter-directed therapy. Blood 119, 4686-4697 (2012).
81. Wang Y D, Li S J, Liao J X. Inhibition of glucose transporter 1 (GLUT1) chemosensitized head and neck cancer cells to cisplatin. Technol Cancer Res Treat 12, 525-535 (2013).
82. Song K, et al. HIF-1alpha and GLUT1 gene expression is associated with chemoresistance of acute myeloid leukemia. Asian Pac J Cancer Prev 15, 1823-1829 (2014).
83. Matsumoto T, Jimi S, Migita K, Takamatsu Y, Hara S. Inhibition of glucose transporter 1 induces apoptosis and sensitizes multiple myeloma cells to conventional chemotherapeutic agents. Leuk Res 41, 103-110 (2016).
84. Jiang T, Zhou M L, Fan J. Inhibition of GLUT-1 expression and the PI3K/Akt pathway to enhance the chemosensitivity of laryngeal carcinoma cells in vitro. Onco Targets Ther 11, 7865-7872 (2018).
85. Wang J, et al. GLUT1 is an AR target contributing to tumor growth and glycolysis in castration-resistant and enzalutamide-resistant prostate cancers. Cancer Lett 485, 45-55 (2020).
86. Gietz R D, Schiestl R H, Willems A R, Woods R A. Studies on the transformation of intact yeast cells by the LiAc/SS-DNA/PEG procedure. Yeast 11, 355-360 (1995).
87. Mulhardt C, Beese E W. 8. DNA analysis. In: Molecular Biology and Genomics). Academic Press (2007).
88. Elia I, et al. Proline metabolism supports metastasis formation and could be inhibited to selectively target metastasizing cancer cells. Nat Commun 8, 15267 (2017).
89. de Koning W, van Dam K. A method for the determination of changes of glycolytic metabolites in yeast on a subsecond time scale using extraction at neutral pH. Anal Biochem 204, 118-123 (1992).
90. Hohmann S, et al. Novel alleles of yeast hexokinase PII with distinct effects on catalytic activity and catabolite repression of SUC2. Microbiology (Reading) 145 (Pt 3), 703-714 (1999).
91. Thomas B J, Rothstein R. Elevated recombination rates in transcriptionally active DNA. Cell 56, 619-630 (1989).

1.-15. (canceled)

16. A method for preventing or treating a cancer or a condition associated with or aggravated by an overactive glycolytic flux, the method comprising administering an inhibitor of hexokinase-dependent glucose carrier-mediated glucose uptake in a subject in need thereof.

17. The method of claim 16, wherein the inhibitor inhibits the hexokinase-dependent glucose uptake by a glucose carrier that is at least one of a mammalian GLUT carrier and a yeast HXT carrier.

18. The method of claim 17, wherein the mammalian GLUT carrier is a class I mammalian GLUT carrier.

19. The method of claim 16, wherein the inhibitor is characterised in at least one of:
a) growth inhibition of A549 lung adenocarcinoma cells grown in a medium with 1 mM glucose at a concentration of the inhibitor of no more than 50 μM; and,
b) restoration of growth on glucose of a tps1Δ yeast strain in a medium containing 2% galactose and 2.5 mM glucose at a concentration of the inhibitor of no more than 100 μM.

20. The method of claim 16, wherein at least one of:
a) the structure of the inhibitor comprises a moiety that resembles the structure of adenosine; and,
b) the inhibitor binds into the ATP-binding domain of a hexokinase-dependent glucose carrier.

21. The method of claim 16, wherein the cancer is a solid tumor or a blood malignancy.

22. The method of claim 16, wherein the cancer is a newly diagnosed cancer that is naive to treatment, a relapsed cancer, a refractory cancer, a relapsed and refractory cancer and/or metastasis of the cancer.

23. The method of claim 21, wherein the inhibitor is used in the prevention and/or treatment of the cancer or metastasis thereof as adjunctive therapy, in combination with one or more treatments selected from the group consisting of: surgery, radiation therapy, chemotherapy and immunotherapy.

24. The method of claim 23, wherein the condition associated with or aggravated by an overactive glycolytic flux is a condition or disease selected from the group consisting of pulmonary hypertension, cardiac hypertrophy, heart failure, atherosclerosis, Alzheimer's diseases, multiple sclerosis, polycystic kidney disease, tuberculosis, diabetic kidney disease and an autoimmune disease.

25. The method of claim 16, wherein the inhibitor is a compound of the general formula (I): 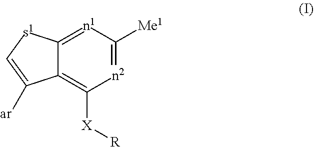
wherein
each of s¹, n¹, and n² is independently chosen from N, O, and S;
Me¹ is a C₁₋₁₀hydrocarbon moiety that is optionally substituted with 1 or 2 alkyl, halogen, or alkoxy moieties;
ar is a 5-10-membered aryl or heteroaryl moiety that is optionally substituted with 1 or 2 alkyl, halogen, or alkoxy moieties;
X is S, NH, or O; and
R is a C₁₋₂₅hydrocarbon moiety that can comprise 0 to 8 heteroatoms and 0 to 3 cyclic moieties.

26. The method of claim 25, wherein the inhibitor is a compound of the general formula (II): 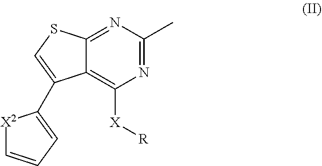
wherein X² is S, NH, or O, and wherein
X is S, NH, or O; and
R is a C1-25hydrocarbon moiety that can comprise 0 to 8 heteroatoms and 0 to 3 cyclic moieties.

27. The method of claim 25, wherein the inhibitor is a compound of the general formula (III): 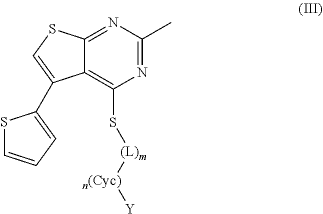
wherein
m is 0, 1, or 2, preferably 0 or 1;
n is 0, 1, or 2, preferably 0 or 1;
L is a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N;
Cyc is a 5 to 10 membered cyclic, heterocyclic, aromatic, or heteroaromatic moiety that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C≡N; and
Y is H or a linear C1-6hydrocarbon that can be interrupted by 0, 1, or 2 heteroatoms, and that can be substituted by 0, 1, or 2 moieties selected from ═O, —O—CH₃, C1-4alkyl, C1-4acyl, —N₃, —NH₂, —OH, trihalomethyl, C5-10aryl, C5-10heteroaryl, and —C—N.

28. The method of claim 25, wherein the inhibitor is a compound of the general formula (Ia) or (Ib) or (Ic):

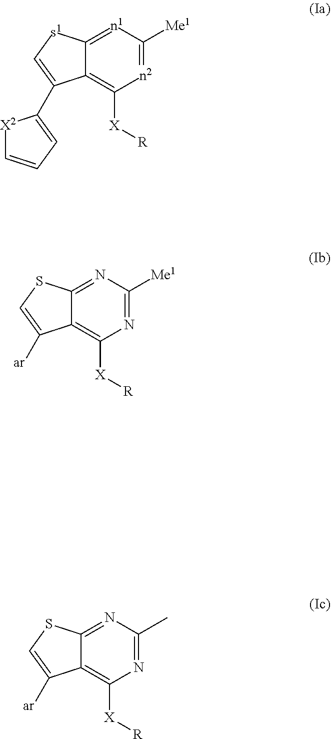

29. The method of claim 28, wherein the inhibitor is a compound of the general formula (IIa):

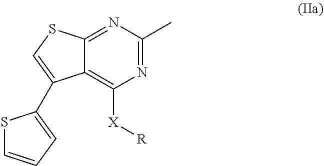

29. The method of claim 25, wherein R is preferably of general formula (R1):

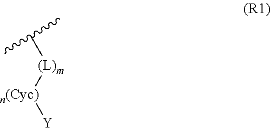

30. The method of claim 29, wherein R is selected from the group consisting of R1-R60 of Table 1.
